(19) United States
(12) Patent Application Publication
(10) Pub. No.: US 2018/0226150 A1
(43) Pub. Date: Aug. 9, 2018

(54) SYSTEMS, DEVICES, AND METHODS FOR EPISODE DETECTION AND EVALUATION WITH VISIT GUIDES, ACTION PLANS AND/OR SCHEDULING INTERFACES

(71) Applicant: ABBOTT DIABETES CARE INC., Alameda, CA (US)

(72) Inventors: Gary A. Hayter, Oakland, CA (US); Michael R. Love, Pleasanton, CA (US); Roy Tsuchida, San Jose, CA (US); Nathan Crouther, San Francisco, CA (US); Daniel M. Bernstein, El Granada, CA (US); Timothy C. Dunn, San Francisco, CA (US); Richard J. Kedziora, Maple Glen, PA (US)

(21) Appl. No.: 15/867,625

(22) Filed: Jan. 10, 2018

Related U.S. Application Data
(60) Provisional application No. 62/597,816, filed on Dec. 12, 2017.
(60) Provisional application No. 62/445,168, filed on Jan. 11, 2017.

(51) Int. Cl. G16H 40/20; A61B 5/145; A61B 5/00; G06K 7/10

(52) U.S. Cl. CPC G16H 40/20; A61B 5/14503; G06K 7/10297; A61B 5/7435; A61B 5/14532; A61B 5/0022

(57) ABSTRACT

Systems, devices, and methods are provided that allow detection of episodes in analyte measurements and the prompting of a subject to self-report possible causes for the episodes and take action in response to episode detection. Correlation of possible causes with detected episodes assists patient behavior modification to reduce the occurrence of episodes. Also provided is function and structure for associating episodes with visits between the subject and a Health Care Provider (HCP) and scheduling interfaces and reports for assisting the HCP in advising numerous patients.

### CROSS-REFERENCE TO RELATED APPLICATIONS

The present application claims priority to and the benefit of U.S. Provisional Patent Application Ser. No. 62/445,168, filed Jan. 11, 2017, and U.S. Provisional Patent Application Ser. No. 62/597,816, filed Dec. 12, 2017, both of which are incorporated by reference herein in their entirety for all purposes.

### FIELD

The subject matter described herein relates to systems, devices, and methods capable of, among others, determining or predicting the occurrence of an episode in analyte data, evaluating the circumstances relating to and potentially causing that episode with a visit guide, and/or utilizing an action plan to reduce the occurrence of episodes through behavioral modification.

### BACKGROUND

A number of systems have been developed for the automatic monitoring of analytes, like glucose, in bodily fluid such as in blood, in interstitial fluid (ISF), dermal fluid of the dermal layer, or in other biological fluid.

These systems can provide a determination of analyte levels, or readings, over time to a health care provider (HCP), a diabetic patient, and/or a caregiver. Knowing the current analyte level and how it may change over time can be useful in determining a course of action for mitigating potentially significant variations in the analyte level, referred to as excursions.

A reduction in the frequency and/or severity of excursions can lessen analyte level variability, and thus is beneficial to the diabetic patient. But diabetics with relatively high analyte variability may find it difficult to treat high analyte level excursions by the administration of a medication, such as insulin, to lower their analyte level, since doing so can increase the risk of low level excursions, due to the large analyte level swings that define high variability. Conversely, treatment of low level excursions through the consumption of food or carbohydrates, to raise the diabetic's analyte level can accompany an increased risk of high level excursions resulting from the subsequent raised analyte level and high variability.

An understanding of what causes, symptoms, and/or treatments impact the occurrence of excursions would be beneficial in reducing their frequency and severity. There are many possible reasons for the glucose excursions that make up glycemic variability: missing bolus and basal dosing, improper bolus timing, missing or improper correction bolus dosing, improper bolus amounts to cover meals, foods that are difficult to adequately cover with rapid acting insulin, irregular strenuous activity, stress or illness, and new medications, for example. For people with inconsistent lifestyles and dietary habits it may be difficult to identify the exact cause or causes of their glycemic variability. Also, even if these causes can be identified, it can be challenging for people to make the required behavioral modifications, similar in nature to the challenges faced by many people who struggle to lose weight.

A number of metrics are available to HCPs and patients to characterize or describe the variation of analyte levels (e.g., averages, medians, percentile variations, variability metrics, risk metrics, rates of change, etc.). Analyte monitoring devices and systems are capable of generating a vast amount of data that can overwhelm and confuse users to the point that little or no insight can be gained as to the reasons driving the occurrence of excursions or high variability. Additionally, recording information that may be beneficial to understanding causation can be burdensome, since it is unclear to the patient what information should be recorded. One of the primary challenges in the investigation of causes of excursions and variability is the disassociation between the analyte level monitoring mechanism, responsible for gathering the data that is the foundation of the analysis, and the relevant conditions leading up to the occurrence of the excursions or in those times contributing most heavily to variability.

For many patients it is vital to have sufficient clinical support to investigate the causes for their glycemic variability. But current clinical techniques for this investigation leave much to be desired. Investigation into those causes and activities is typically performed by a question and answer session between the diabetic and the HCP, which may occur days or weeks after the fact. Such sessions are disadvantageous because the diabetics often cannot remember the actions and conditions leading up to the excursion, or those actions and conditions are unknown because the diabetic was not aware of the need to track those conditions, e.g., by investigating the dose of recent administrations of medication, the carbohydrate content of recently consumed foods, the duration of recent exercise, activity, or sleep, and the like. Further, when questioned directly by an HCP, diabetics can feel obligated to supply answers that make the diabetic appear to be leading a healthier lifestyle than may, in fact, be true.

Also, even if causes are identified, it is important that the clinical support assist the patient in determining the appropriate action plan that defines the behavior change that they can take to address the cause, and to reinforce this behavior modification. However, the level of clinical support to accomplish this has been prohibitive for most patients and health plans. Therefore, there is a need for the tools that a clinician will use to be efficient; that is, tools are needed that minimize the amount of time required by using automated evaluation and guidance, and focusing on only those activities that efficiently help patients reach their glucose management goals.

For these and other reasons, needs for improved variability and excursion monitoring, investigation, and evaluation exist.

### SUMMARY

Provided herein are a number of example embodiments directed to systems, devices, and methods for investigating the reasons that cause the occurrence of analyte excursions and/or relatively high analyte variability. For example, to assist in the reduction of analyte variability, these embodiments can identify and/or investigate the occurrence of analyte episodes that contribute to excessive variability. In many embodiments, these episodes can include the occurrence of an actual analyte excursion beyond clinically safe levels, but can include other variations that may not typically qualify as an excursion.

For example, certain embodiments can provide for the monitoring of a diabetic's analyte levels, detecting whether one or more analyte-related episodes have occurred (or predicting their future occurrence) based on the monitored analyte levels, prompting the diabetic for information about the episode in an improved fashion, prompting the diabetic to take a particular action in response to the detected episode, and investigating (or enabling the investigation) of conditions that may contribute to the episode and the efficacy of actions taken in response to the episode. Many of these embodiments can utilize an in vivo sensor control device that receives measurements of analyte levels from a sensor in the diabetic's body, a reader device that receives the measurement data from the sensor control device, and a monitoring application that resides on the reader device and analyzes the measurement data.

Example embodiments of episode investigative software (EIS) can be provided on the reader device, e.g., as a smartphone app, and/or on one or more other computing devices, for example, via a web server, that facilitates the collection of information from the diabetic subject and performs or enables the subsequent investigation into the underlying root causes and conditions of episodes and variability. Embodiments of the EIS can generate a visit guide menu and allow a user to associated analyte and episodic data with a particular visit between the diabetic subject and the health care professional (HCP). Reports can be generated with the EIS that are linked directly from the visit guide, enabling the HCP and subject to more readily evaluate progress in treating episodic occurrences and trends.

Embodiments of the EIS can also be used to assist HCP's in interfacing with patients in a crowded scheduling environment. Scheduling interfaces are described that permit HCPs to readily understand their upcoming schedule in the context of a visit sequence corresponding to the EIS. Many various embodiments of reports are provided that permit the HCP ready access to relevant glycemic data of each patient before and after a previous intervention or interventions. These reports can suggest therapies and provide access to interfaces for creating or editing an action plan for the patient. A number of related embodiments are also disclosed.

Other systems, devices, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is a high level diagram depicting an example embodiment of an analyte monitoring system for real-time analyte (e.g., glucose) measurement, data acquisition and/or processing using a monitoring application on a reader device.

FIG. 1B is a block schematic depicting an example embodiment of a reader device.

FIG. 1C is a block schematic depicting an example embodiment of a sensor control device.

FIGS. 2-9C are diagrams depicting example embodiments of graphical user interface screens that can be displayed on the display of a reader device or other computing device executing or accessing an example embodiment of the episode investigative software.

FIG. 10 is a flow diagram depicting an example embodiment of automatic configuration of the episode investigative software.

FIGS. 11-15 are diagrams depicting example embodiments of graphical user interface screens that can be displayed on the display of a reader device or other computing device executing or accessing an example embodiment of the episode investigative software.

FIGS. 16-22B are diagrams depicting example embodiments of graphical user interface screens that can be displayed on the display of a computing device executing or accessing an example embodiment of the episode investigative software.

FIGS. 22C-D are flow diagrams depicting example methods performed by instructions implementing the episode investigative software.

FIGS. 23-24 are diagrams depicting example embodiments of graphical user interface screens that can be displayed on the display of a computing device executing or accessing an example embodiment of the episode investigative software.

FIG. 25 is a flow diagram depicting an example embodiment of a method of treatment of a patient with the assistance of the episode investigative software.

FIGS. 26-51A are diagrams depicting example embodiments of graphical user interface screens that can be displayed on the display of a computing device executing or accessing an example embodiment of the episode investigative software.

FIGS. 51B-51G are diagrams depicting example embodiments of graphical user interface screens that can be displayed on the display of a reader device or other computing device executing or accessing an example embodiment of the episode investigative software.

FIGS. 52-60 are diagrams depicting example embodiments of graphical user interface screens that can be displayed on the display of a computing device executing or accessing an example embodiment of the episode investigative software.

FIG. 61 is a conceptual block diagram of an example embodiment of a combined EIS-expert system.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

### Example Embodiments of Analyte Monitoring Systems

As mentioned, a number of systems have been developed for the automatic monitoring of analyte(s), including but not limited to glucose, in bodily fluid such as in blood, in interstitial fluid (ISF), dermal fluid of the dermal layer, or in other biological fluid. Some of these systems are configured so that at least a portion of a sensor is positioned in a patient, e.g., in a blood vessel or in the subcutaneous tissue or dermal layer of a patient, to obtain information about at least one analyte of the body. As such, these systems can be referred to as “in vivo” analyte monitoring systems.

In vivo analyte monitoring systems include “Continuous Analyte Monitoring” systems (or “Continuous Glucose Monitoring” systems) that can transfer data by transmission of the data from a sensor control device to a reader device continuously with or without prompting, e.g., automatically according to a schedule. In vivo analyte monitoring systems also include “Flash Analyte Monitoring” systems (or “Flash Glucose Monitoring” systems or simply “Flash” systems) that can transfer data from a sensor control device in response to a user-initiated request (e.g., a scan) for data issued by a reader device, such as with Radio Frequency Identification (RFID), Near Field Communication (NFC), or another protocol. An in vivo analyte monitoring system has been developed that also can operate without the need for finger stick calibration.

In vivo analyte monitoring systems can be differentiated from “in vitro” systems that contact a biological sample outside of the body (or rather “ex vivo”) and that typically include a meter device that has a port for receiving an analyte test strip carrying bodily fluid of the patient, which can be analyzed to determine the patient's blood sugar level. While in many of the present embodiments the monitoring is accomplished in vivo, the embodiments disclosed herein can be used with in vivo analyte monitoring systems that incorporate in vitro capability, as well has purely in vitro or ex vivo analyte monitoring systems.

The sensor can be part of the sensor control device that resides on the body of the patient and contains the electronics and power supply that enable and control the analyte sensing. The sensor control device, and variations thereof, also can be referred to as a “sensor control unit,” a “sensor electronics” device or unit, an “on-body electronics” device or unit, an “on-body” device or unit, or a “sensor data communication” device or unit, to name a few.

In vivo monitoring systems also can include a device that receives sensed analyte data from the sensor control device and processes and/or displays that sensed analyte data, in any number of forms, to the user. This device, and variations thereof, can be referred to as a “reader device” (or simply a “reader”), “handheld electronics” (or a handheld), a “portable data processing” device or unit, a “data receiver,” a “receiver” device or unit (or simply a receiver), or a “remote” device or unit, to name a few. Other devices such as personal computers have also been utilized with or incorporated into in vivo and in vitro monitoring systems.

The embodiments described herein can be used to monitor and/or process information regarding any number of one or more different analytes. Analytes that may be monitored include, but are not limited to, acetyl choline, amylase, bilirubin, cholesterol, chorionic gonadotropin, glycosylated hemoglobin (HbA1c), creatine kinase (e.g., CK-MB), creatine, creatinine, DNA, fructosamine, glucose, glucose derivatives, glutamine, growth hormones, hormones, ketones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In embodiments that monitor more than one analyte, the analytes may be monitored at the same or different times.

Many of the example embodiments described herein make reference to the monitoring of glucose; however, such references recognize only that glucose is a commonly monitored analyte and those references should not be interpreted as excluding operation with analytes other than glucose.

An in vivo system manufacturer can provide patients with both the sensor control device and the corresponding reader device; in some cases, the two can be sold as a set. The sensor control device can have a limited lifespan and can be replaced periodically (e.g., every two weeks), but the reader device can be used for a significantly longer period of time and may be reusable with each new replacement sensor control device.

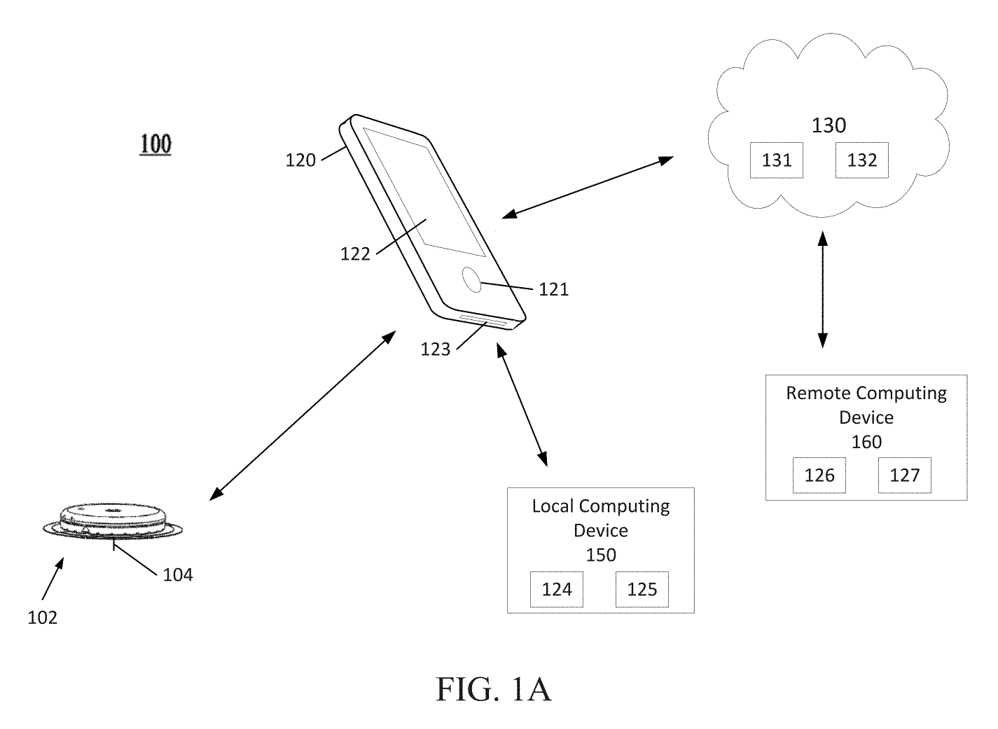

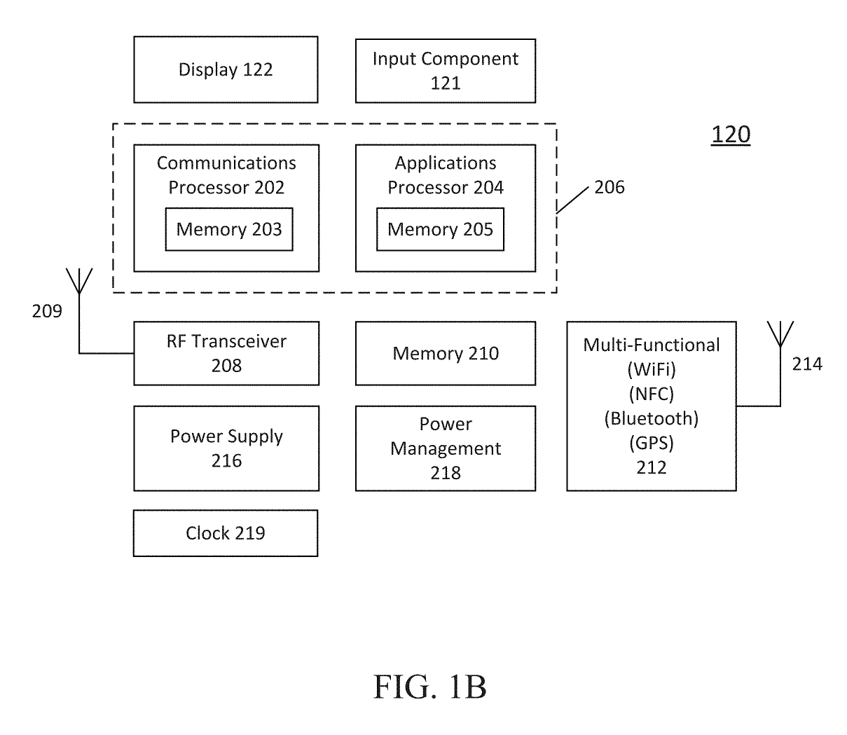

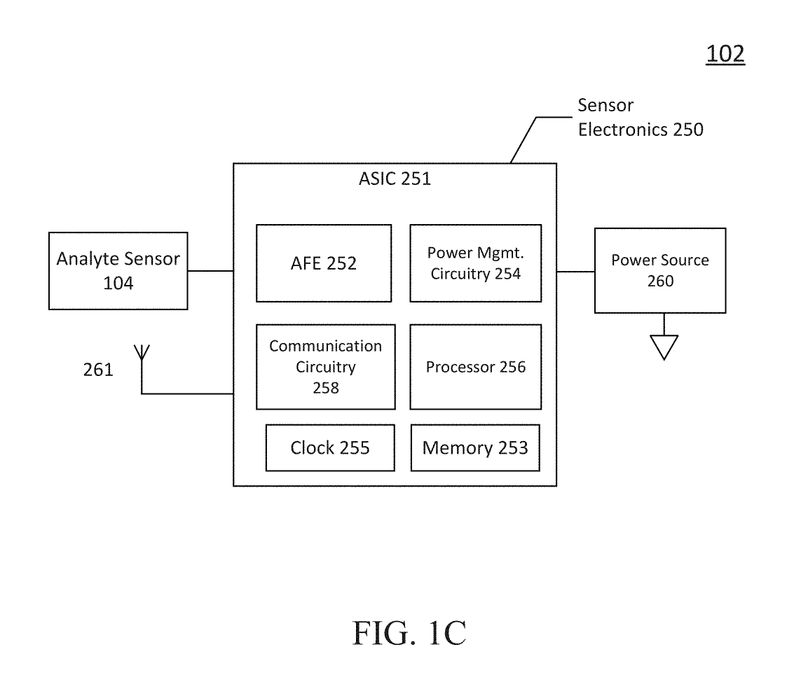

An example embodiment of an analyte monitoring system 100 is shown in FIG. 1A. System 100 can include one or more of a measuring device, such as a sensor control device 102 with an analyte sensor 104, and a reader device 120, such as a handheld smartphone running a software application, such as a glucose monitoring application 140. One or more instances of the episode investigative software (EIS) can be stored and executed within system 100.

The EIS can be stored and executed on a network server 130 (e.g., in the cloud) that is accessible by any computing device having an internet connection and an internet browser, such as reader device 120, local computing device 150, and/or remotely located computing device 160. Here, a device is local if it is accessible in the same vicinity (e.g., the same office, home, or building) as reader device 120, and a device is remote if it is located in a different vicinity (e.g., the office of the patient's HCP, etc.) than reader device 120. Alternatively, the EIS can be locally stored on and executed on reader device 120, local computing device 150, and/or remote computing device 160. Examples of local and remote computing devices can include a personal computer (PC), laptop computer, tablet computer, personal digital assistant (PDA), workstation, wearable smart device (GOOGLE GLASS, APPLE WATCH), and others.

Each device that can store and/or execute software includes non-transitory memory for storing the software and processing circuitry communicatively coupled with the non-transitory memory for executing the software. These devices also include the appropriate communications interface for communication with other devices (e.g., a network communications port, etc.). For example, server 130 includes processing circuitry 131 communicatively coupled with non-transitory memory 132, local computing device 150 includes processing circuitry 124 communicatively coupled with non-transitory memory 125, and remote computing device 160 includes processing circuitry 126 communicatively coupled with non-transitory memory 127. Because the EIS can be stored and executed on a number of combinations of devices, server 130, local computing device 150, and remote computing device can each be selectively omitted.

As shown in FIG. 1A, the components can be in wireless communication with each other using a wireless communication protocol such as, for example, NFC, RFID, Wi-Fi, Bluetooth, Bluetooth Low Energy, or proprietary protocols. Each of the components can also be in direct communication through a wired link (e.g., USB) or indirectly through a distributed wired network (e.g., TCP/IP).

Data obtained from sensor control device 102 and patient-supplied data (described below) is stored in a location accessible to the EIS, e.g., in reader device 120, network server 130, local computing device 150, and/or remote computing device 160. The data can be uploaded from reader device 120. The stored data can be processed and/or analyzed by the EIS to assist diabetics, patients, interested people (e.g., parents, guardians, caretakers), health care professionals (HCPs) or any users in identifying patterns and reasons that may cause episodes, which in turn can lead to improved analyte level control (e.g., reduced variability).

The EIS can correlate diabetic actions, lifestyle, and/or behavior with glucose levels, thereby reducing the burden on the user to sort out the effects. The EIS can use clinically-informed algorithms to search glucose data acquired for an individual patient and the patient's recorded behaviors (or a plurality of patients and patient records) to reveal patterns that affect glucose levels. In some embodiments, the EIS can include instructions that: 1) define episodes of interest, 2) select a kernel episode for the search routine, 3) construct an episode in close proximity to the kernel, 4) associate one or more episodes with a diabetes self-care behavior, and 5) cause the display of the findings of the search algorithms. In other embodiments, the EIS can include instructions that: 1) define episodes of interest, 2) select a kernel episode for the search routine, 3) construct an episode chain, which is a sequence of episodes (including the kernel episode) and logical rules for the inclusion or exclusion of episodes in close proximity to the kernel, 4) associate one or more episode chains with a diabetes self-care behavior, and 5) cause the display of the findings of the search algorithms.

Additional examples of episode investigative systems, software, and algorithms that are usable with, or in place of, the EIS systems, software, and algorithms described herein, are set forth in U.S. Patent Application Publication No. 2014/0350369, U.S. Patent Application Publication No. 2014/0088392, and U.S. Patent Application Publication No. 2014/0088393, all of which are incorporated herein by reference in their entirety and for all purposes.

Referring back to FIG. 1A, sensor control device 102 is configured to receive glucose level readings from an in vivo positionable sensor 104, e.g., a subcutaneous sensor, dermal sensor, blood vessel sensor, and the like. In other embodiments, the sensor can be positioned ex vivo but can monitor the analyte level in vivo, such as certain optical sensors. Sensor control device 102 causes sensor 104 to repeatedly sense glucose levels, either according to a predetermined schedule or on an ad hoc basis, and reads one or more signals representative of the glucose level from sensor 104. These glucose level readings may be stored in sensor control device 102 and transferred to reader device 120 in batches at predetermined times, on-demand, or immediately after a glucose level reading is obtained.

Non-limiting examples of reader devices 120 can include the meter device of an ex-vivo monitoring system, the reader device of an in-vivo monitoring system, combinations of the two such as an in vivo reader operating with a test strip port and meter functionality, and various other devices such as smartphones, tablets, proprietary readers, other computing devices, etc. Reader device 120 can include one or more software applications that perform various program routines, such as communicating with another device executing the EIS, displaying data in various fashions, receiving input from the user (e.g., HCP, caregiver, and/or diabetic patient) to help the diabetic manage their diabetes, and detecting episodes. Reader device 120 can include buttons, such as a keyboard, for example, used for entering information. In addition, or alternatively, reader device 120 can include virtual buttons, such as a touch screen configured to present a virtual keyboard. Reader device 120 can include a microphone for receiving vocal input that is then processed with speech recognition software.

Reader device 120 can also include or be integrated with a drug (e.g., insulin, etc.) delivery device such that they, e.g., share a common housing. The drug delivery hardware can include a reservoir to store the drug and a pump that can be connectable to transfer tubing and an infusion cannula for administering the drug from the reservoir, through the tubing and into the diabetic's body by way of the cannula inserted therein.

FIG. 1B is a block diagram of an example embodiment of a reader device 120 in the form of a smartphone. Here, reader device 120 includes an input component 121, display 122, and processing circuitry (or hardware) 206, which can include one or more processors, microprocessors, controllers, and/or microcontrollers, each of which can be a discrete chip or distributed amongst (and a portion of) a number of different chips. Processing hardware 206 can include a communications processor 202 having on-board memory 203 and an applications processor 204 having on-board memory 205. Additional processors can and will likely be present. Reader device 120 further includes an RF transceiver 208 coupled with an RF antenna 209, a memory 210, multi-functional circuitry 212 with one or more associated antennas 214, a power supply 216, and power management circuitry 218. FIG. 1B is an abbreviated representation of the internal components of a smartphone, and other hardware and functionality (e.g., codecs, drivers, glue logic, etc.) can of course be included.

Communications processor 202 can interface with RF transceiver 208 and perform analog-to-digital conversions, encoding and decoding, digital signal processing and other functions that facilitate the conversion of voice, video, and data signals into a format (e.g., in-phase and quadrature) suitable for provision to RF transceiver 208, which can then transmit the signals wirelessly. Communications processor 202 can also interface with RF transceiver 208 to perform the reverse functions necessary to receive a wireless transmission and convert it into digital data, voice, and video.

Applications processor 204 can be adapted to execute the operating system and any software applications that reside on reader device 120, process video and graphics, and perform those other functions not related to the processing of communications transmitted and received over RF antenna 209. Any number of applications can be running on reader device 120 at any one time, and will typically include one or more applications that are related to a diabetes monitoring regime, in addition to the other commonly used applications that are unrelated to such a regime, e.g., email, calendar, weather, etc.

Memory 210 can be shared by one or more the various functional units present within reader device 120, or can be distributed amongst two or more of them (e.g., as separate memories present within different chips). Memory 210 can also be a separate chip of its own. Memory 210 is non-transitory, and can be volatile (e.g., RAM, etc.) and/or non-volatile memory (e.g., ROM, flash memory, F-RAM, etc.).

Multi-functional circuitry 212 can be implemented as one or more chips and/or components, including communication circuitry, that perform other functions such as local wireless communications (e.g., Wi-Fi, Bluetooth, Bluetooth Low Energy) and determining the geographic position of reader device 120 (e.g., global positioning system (GPS) hardware). One or more other antennas 214 are associated with both the functional circuitry 212 as needed.

Power supply 216 can include one or more batteries, which can be rechargeable or single-use disposable batteries. Power management circuitry 218 can regulate battery charging and power supply monitoring, boost power, perform DC conversions, and the like. As mentioned, reader device 120 may also include one or more data communication ports such as USB port (or connector) or RS-232 port (or any other wired communication ports) for data communication with a remote terminal 170, or sensor control device 102, to name a few. A network syncing clock 219 is also present that can provide the system time and include, for example, an RC or crystal oscillator and associated clock buffers and distribution circuitry.

FIG. 1C is a block schematic diagram depicting an example embodiment of sensor control device 102 having analyte sensor 104 and sensor electronics 250 (including analyte monitoring circuitry). Although any number of chips can be used, here the majority of the sensor electronics 250 are incorporated on a single semiconductor chip 251 that can be, e.g., a custom application specific integrated circuit (ASIC). Shown within ASIC 201 are several high-level functional units, including an analog front end (AFE) 252, power management circuitry 254, processor 256, and communication circuitry 258 (which can be implemented as a transmitter, receiver, transceiver, passive circuit, or otherwise according to the communication protocol). In this embodiment, both AFE 252 and processor 256 are used as analyte monitoring circuitry, but in other embodiments either circuit can perform the analyte monitoring function. Processor 256 can include one or more processors, microprocessors, controllers, and/or microcontrollers.

A non-transitory memory 253 is also included within ASIC 251 and can be shared by the various functional units present within ASIC 251, or can be distributed amongst two or more of them. Memory 253 can be volatile and/or non-volatile memory. In this embodiment, ASIC 251 is coupled with power source 260, which can be a coin cell battery, or the like. AFE 252 interfaces with in vivo analyte sensor 104 and receives measurement data therefrom and outputs the data to processor 256 in digital form, which in turn processes the data to arrive at the end-result analyte discrete and trend values, etc. This data can then be provided to communication circuitry 258 for sending, by way of antenna 261, to reader device 120 (not shown) where further processing can be performed by, e.g., the sensor interface application.

A clock 255 is also present that can be, for example, an RC or crystal oscillator. Clock 255 can be a monotonic clock that moves forward at constant rate (subject to environmental drift) and continues for the entire life of the sensor without interruption. In some embodiments, sensor control device 102 can keep time by generating an interrupt after a predetermined number of seconds (e.g., every second, or every minute etc.), where the interrupt increments a software or hardware counter. The counter value reflects the number of predetermined time spans that have passed since the sensor was activated. For example, if the interrupt is generated every minute, then the counter value reflects the number of minutes that have passed since activation of sensor control device 102. The functional components of ASIC 251 can also be distributed amongst two or more discrete semiconductor chips.

U.S. Patent Application Publ. No. 2011/0213225 (the '225 Publication) describes variants of sensor control device 102 and reader device 120, as well as other components of an in vivo-based analyte monitoring system, all of which are suitable for use with the system, device, and method embodiments set forth herein. As such, the '225 Publication is incorporated by reference herein in its entirety for all purposes.

Example Embodiments with Episode Investigative Software (EIS)

The EIS can be utilized to detect prior, current, and/or future episodes. The term episode, as used herein, does not refer to every variation of an analyte level but rather those variations that significantly contribute to the subject's analyte variability, such as high glucose and low glucose peaks. Episodes can also include glucose excursions or events that are clinically significant, such as a rapid rise or rapid fall in the glucose level. Different conditions can be used to qualify a set of analyte level measurements as an episode, including the analyte measurement's magnitude, the rate of change between sequential analyte measurements or other groups of measurements that are close in time, the number of analyte measurements (or duration of time) in which a threshold magnitude is violated, the violation of a threshold by the area of an integral of a sequence of analyte measurements, any combination thereof, and others. Such episodic detection conditions are described in U.S. Publication No. 2013/0085358, which is incorporated by reference herein in its entirety and for all purposes.

As previously stated, the EIS can be executed on a number of different devices, including mobile devices with sensor interface capability (e.g., reader device 120) or mobile devices without a sensor interface capability (e.g., a typical tablet or mobile phone).

Alternatively, the EIS can be executed partially on the electronics co-located with sensor 104, communicating data that confirms the occurrence of each episode to a user interface (UI)-capable device such as a mobile device, personal computer (PC), and the like. In certain embodiments sensor control device 102 performs a substantial amount of algorithmic processing on the raw collected measurement data to arrive at analyte levels in a form that is readily made displayable to the user. In these embodiments sensor control device 102 can also determine if predetermined conditions for the occurrence of an episode are et by the determined analyte levels. The sensor control device 102 can then transmit the confirmation that an episode has occurred (and any desired information about that episode such as type, time, location, etc.) and optionally the underlying measurement data as well.

The EIS can be stored in the non-transitory memory of the device (e.g., 120, 130, 150, 160) executing the software (e.g., as a plurality of instructions). The EIS can be executed by the processing circuitry of the device. To the extent user input is provided (e.g., the entry of text, a mouse click, a touch selection, vocal input, etc.), it is done so by way of a user interface 121, e.g., a touchscreen or user input of reader device 120, and the user input is received and read (or interpreted) by the processing circuitry, which then causes the appropriate action to be taken (e.g., causing a new screen to be displayed, causing a check mark to be displayed, modifying a pick list, etc.). User input can be entered in a tactile fashion (e.g., by touching) or in a vocal fashion (e.g., into a microphone and processed with speech recognition software).

Reader device 120 in the embodiment of FIG. 1A can be a smartphone running one or more downloadable software applications, commonly referred to as “apps.” The EIS can be configured and distributed as such a downloadable app. The EIS can also be resident software installed directly on reader device 120 at time of manufacture (prior to distribution or sale). The EIS can include the software instructions for interfacing with sensor control device 102 and processing data received from sensor control device 102 into a user-readable value representative of the user's glucose level (for example, applying algorithms to and otherwise processing raw sensor data to determine an actual analyte level that can be displayed to and interpreted by the diabetic or other user). Alternatively, the EIS can operate with a separate software application responsible for communicating with sensor control device 102 and receiving and processing the raw data, such as a glucose monitoring application. That glucose monitoring application can similarly be configured and distributed as a downloadable app, or as factory-installed resident software.

Example Embodiments of Mobile Devices with Episode Investigative Software (EIS)

Although not limited to such, for ease of description, with respect to reader device 120, the EIS will be described as a downloadable app having the glucose monitoring instructions incorporated therein.

Reader device 120 can receive glucose level readings automatically or can request glucose level readings from sensor control device 102 and can make them available to the EIS or otherwise transmit the readings to the device executing the EIS. Reader device 120 can process the readings and send alerts and/or prompts to the patient based on episodes identified by the EIS. Reader device 120 can display one or more of rates of analyte change, reports, tables, graphs, and other representations of processed glucose level readings and inputted behaviors (meals, insulin, etc.) generated by the software on reader device 120 or from the EIS for use by the HCP and patient.

Reader device 120 can receive glucose measurements directly or indirectly (in the case of a primary/secondary receiver system) from sensor control device 102. Additionally or alternatively, in some embodiments, in particular, those that include an ex vivo or non-sensor measuring device, the glucose measurement can be derived from a bodily fluid sample on a test strip, or even manually input into reader device 120 by the patient, e.g., a blood glucose reading. Glucose measurements, whether manually input or obtained from sensor control device 102, can be transferred to the EIS.

Sensor control device 102 can transfer data to reader device 120 in any manner suitable for the implementation. In some embodiments, sensor control device can be used in a continuous mode, with glucose level readings being repeatedly collected and sent by sensor control device 102 to reader device 120 every few seconds, every few minutes (e.g., every 1, 5, 10, 15 minutes), at other regular or irregular intervals, or the like. The transmissions can be asynchronous (e.g., transmitted without a received prompt or query for data) or synchronous (e.g., transmitted after a prompt or query is received). In some embodiments, prompts or queries for data are sent automatically from reader device 120 to sensor control device 102 without initiation by the patient, while in other embodiments the patient can obtain glucose level measurements manually, or “on-demand,” with reader device 120 by a patient-initiated request for data (e.g., an NFC scan) from sensor control device 102. Sensor control device 102 can include sufficient storage memory (e.g., sufficient for 8, 12, 24, or 48 hours of data, or for the entire lifetime of the sensor control device 102) to hold multiple automatically gathered glucose level measurements until transferred to reader device 120. The transferred data can include all glucose readings that are stored in memory in sensor control device 102, or a subset, such as those that have not already been transferred to reader device 120, or those that have been collected in a recent period of time, or a predetermined number of recent measurements (e.g., two or three, etc.).

The EIS can be configured as a “masked” version or an “unmasked” version. The masked version can be used for a period of time to establish a baseline, for example, a multi-day period such as 3 days, one week, two weeks, and others. The masked version can allow the patient limited access to glucose readings and other information or features of the application. The patient can obtain glucose data from sensor control device 102 to record glucose levels for later assessment by the HCP, however, access to that glucose information is restricted. For example, in some embodiments, the patient is not shown any glucose level reading during the masked period of time, but is allowed to see the nature or type of various episodes (e.g., high glucose episode, low glucose episode, etc.). In other embodiments, the patient is not shown any glucose reading nor is the patient shown any information about the nature or type of episode during the masked period of time. For example, the patient may be informed only that “an episode has occurred” without any further information about the episode.

The masked version encourages the patient to follow normal eating, exercising, and medicating behaviors free of any influence that knowledge of glucose levels or episode types may cause. Receipt of data from sensor control device 102 at frequent intervals, for example, at least once every eight hours or other length of time, is performed to obtain multiple glucose level readings. The EIS can prompt the patient to scan sensor control device 102 if necessary, or alternatively, a reminder application can be used.

The unmasked version of the EIS allows patient access to more, sometimes to all, features. In certain embodiments, masked and unmasked versions of the application do not exist concurrently on reader device 120. In other embodiments, masked and unmasked versions can exist concurrently on reader device 120. In yet other embodiments, the two versions can exist within one application and a software switch accessible by the HCP can be used to switch between one version and the other.

As discussed, the EIS can be installed on reader device 120 in any manner. The glucose monitoring application can be installed on smartphones running various operating systems, for example, ANDROID, iOS, or others. Installation can be accomplished using standard installation methods. For example, installation on an ANDROID-based smartphone can be done by e-mailing an apk file to reader device 120 and executing the file.

After installation of the EIS (if necessary), the patient and reader device 120 can be registered on server 130 and associated with one another. The patient may be given a temporary username or a permanent username may be assigned. The username should be unique, and if a temporary username is provided, when the patient requests a permanent username, it should be vetted to ensure uniqueness. The username may be an e-mail address or other unique set of alphanumeric symbols. Reader device 120 may be identified by any unique set of identifiers associated with reader device 120, for example, an IMEI number or a MAC address.

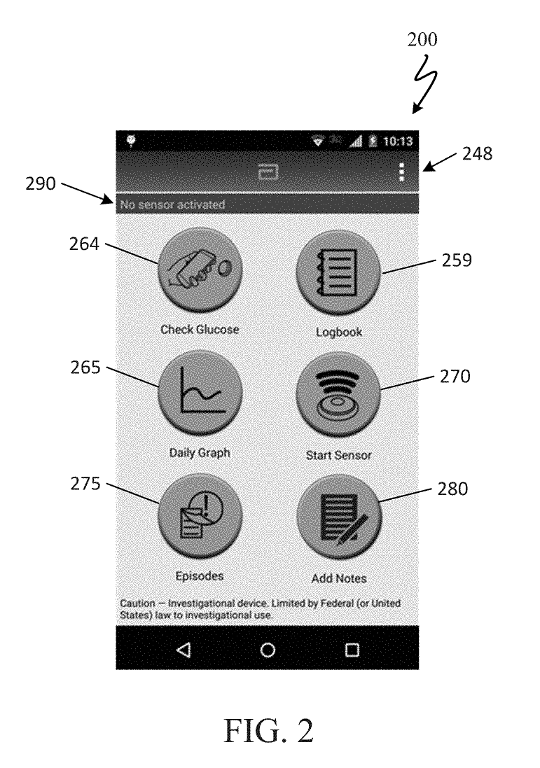

FIG. 2 depicts an example embodiment of a graphical user interface (GUI) display screen 200 that can be displayed to the user upon activation of the EIS on display 122 of reader device 120, which in this embodiment is a touch screen. Here, display screen 200 is a home page 200 that can include multiple user selectable fields. Selectable fields are regions of the display that can be selected by the user, such as with touch in the case of a touchscreen, selection with a mouse cursor and click or touch pad tap, selection with a voice control, and other manners of selection. The selectable field as displayed on the screen can be a button, a tab, selectable text (e.g., a hyperlink), a free text entry field, a geometric shape such as an arrow indicating a drop down menu, a check box or circle, and the like. Selection of the field can result in the display of an entirely new screen, the display of a pop-up window over the current screen, the display of a drop-down menu, can allow the entry of text into a free text field, can result in selection of a displayed option, and the like. For ease of reference these fields will be referred to herein on certain occasions as tabs or buttons.

Home page 200 can include one or more of a menu button 248, a check glucose button 264, a logbook button 259, a daily graph button 265, a start sensor button 270, an episodes button 275, an add notes button 280, and a sensor status area 290. Sensor status area 290 indicates whether sensor 104 has been activated, has expired, or the length of time remaining before expiration. Example functions of each home page button are described below.

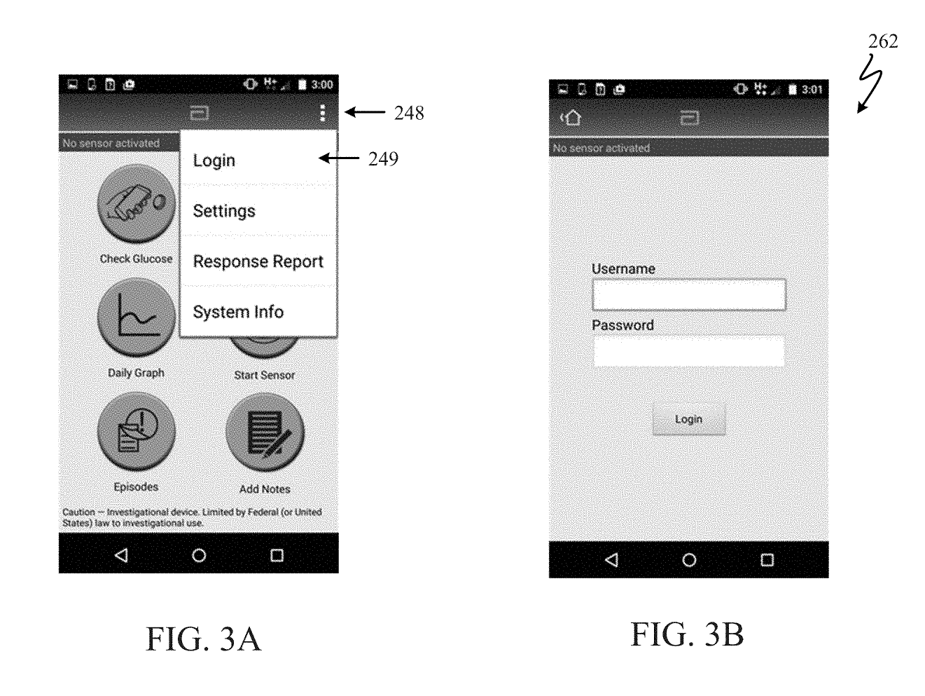

To log into server 130, the menu button 248 is clicked and the Login field 249 is selected, as shown in the example embodiment of FIG. 3A, which causes the display of login screen 262, shown in the example embodiment of FIG. 3B. The user can then enter their username and password to log into server 130. In some embodiments, the user is first logged in before the EIS will automatically upload data to server 130. After logging in, pressing menu button 248 results in different menu choices being presented, as will be described below.

Referring back to FIG. 2, start sensor button 270 can be used to activate sensor 104 after sensor control device 102 is applied to the patient. When sensor 104 is changed, e.g., after about 14 days, the activation step is repeated. In embodiments where reader device 120 communicates with sensor control device 102 over an NFC link, reader device 120 can be held within a close proximity communication range, for example, approximately 3 inches or less, of sensor control device 102. Reader device 120 can be repositioned around sensor control device 102, if needed until communication is established, signified by reader device 120 generating a sound and/or vibration or other connection indicator. Once the sound or vibration occurs, reader device 120 can be held in place for an amount of time, for example, about 1-2 seconds, until another sound and/or vibration occurs indicating that sensor 104 (or sensor control device 102) has been activated. In some embodiments, glucose level measurements will not be made or reported to reader device 120 until the expiration of a predetermined amount of time after activation that allows the proper initialization of sensor 104. In some embodiments, this “warm-up period” is 60 minutes, although the length of time can depend on the sensor itself and may be longer or shorter, or may not be required at all.

Still referring to FIG. 2, after sensor 104 is activated, glucose levels can be checked using the EIS. When check glucose button 264 is selected, reader device 120 obtains one or more glucose measurements from sensor control device 102 using one of the communication methods described herein, such as an on-demand method. Additionally, the readings can be sent automatically from reader device 120 to server 130.

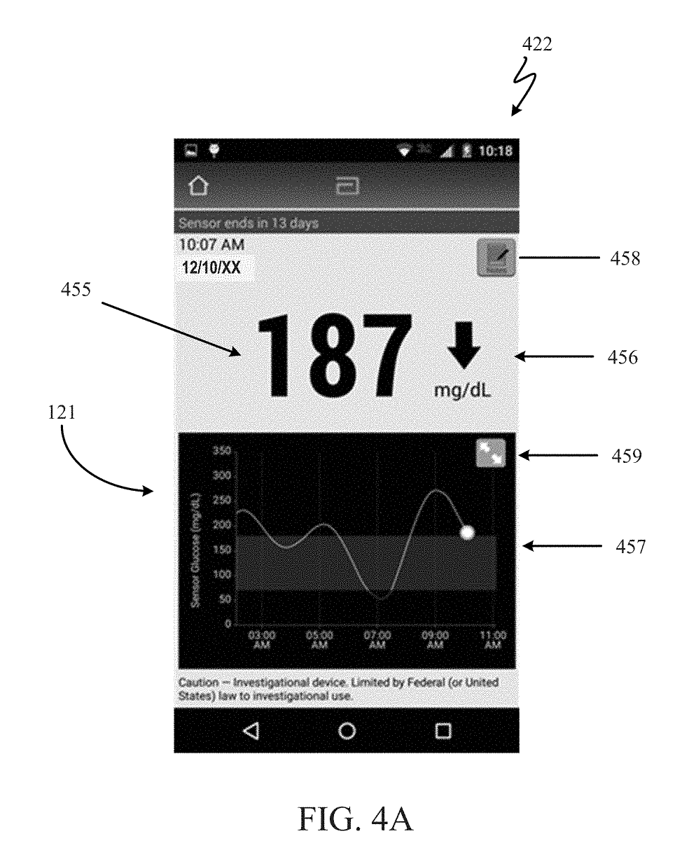

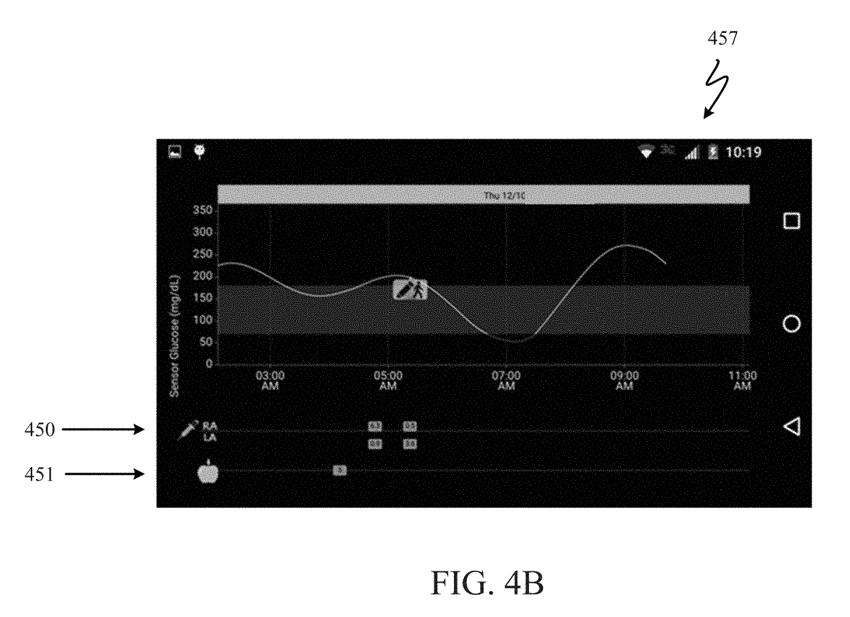

After the glucose level readings are transferred, screen 121 of reader device 120 can automatically display a result screen 422 as shown in the example embodiment of FIG. 4A. Result screen 422 can include one or more of a display of the current glucose level reading 455, a trending arrow 456 showing the direction and/or rate in which glucose level readings are changing, and a glucose graph 457. A notes button 458 also can be presented on the result screen 422. If the result is more than a predetermined amount of time, e.g., 3 minutes old in some instances, a warning message can appear indicating that the results are not current.

Glucose reading 455 can be the current glucose level reading up to a predetermined level, e.g., 350 mg/dl. Readings greater than the predetermined level may be sticky, in that they may be displayed as the predetermined level. Trending arrow 456 can be one of a plurality of indicators, e.g., five different, relative arrows. In certain embodiments, an arrow pointing up can indicate glucose level readings are quickly rising at a rate greater than a predetermined high rate, e.g., 2 mg/dl per minute. An arrow pointing up and to the right can indicate glucose level readings are rising at a predetermined moderate rate, e.g., between 1 and 2 mg/dl per minute. If glucose level readings are changing upwardly or downwardly at a predetermined low rate, e.g., less than 1 mg/dl per minute, the arrow can point to the right. An arrow pointing down and to the right can indicate glucose level readings are falling at a predetermined moderate rate, e.g., between 1 and 2 mg/dl per minute, and an arrow pointing down can indicate glucose level readings are quickly falling at a predetermined rate, e.g., more than 2 mg/dl per minute.

Glucose graph 457 can display measured glucose level readings over a predetermined time range, for example, over the past 8 hours, and the time range can be increased or decreased using the zoom button 459 or by using standard 2-finger zoom techniques of reader device 120. The time range can be shifted by dragging the plot left or right with a finger. A symbol, for example, a clock symbol, can appear in the graph to indicate that the reader device's time was changed. A change in time can result in gaps in the graph, overlapping readings, or hidden data that the HCP, patient, and/or caregiver should be made aware of.

FIG. 4B depicts an example embodiment of glucose graph 457 after depressing zoom button 459. Beneath graph 457 is an medication timeline 450 indicating times and amounts of rapid acting insulin (top) and long acting insulin (bottom) that were administered over the displayed time period, with numerical indicators conveying the respective dose amount and aligned appropriately to convey the associated time of dose administration. Beneath timeline 450 is a meal timeline 451 indicating times and amounts of meals or food that was consumed over the displayed time period, with a numerical indicator conveying the consumed carbohydrate amount and aligned appropriately to convey the associated time of consumption

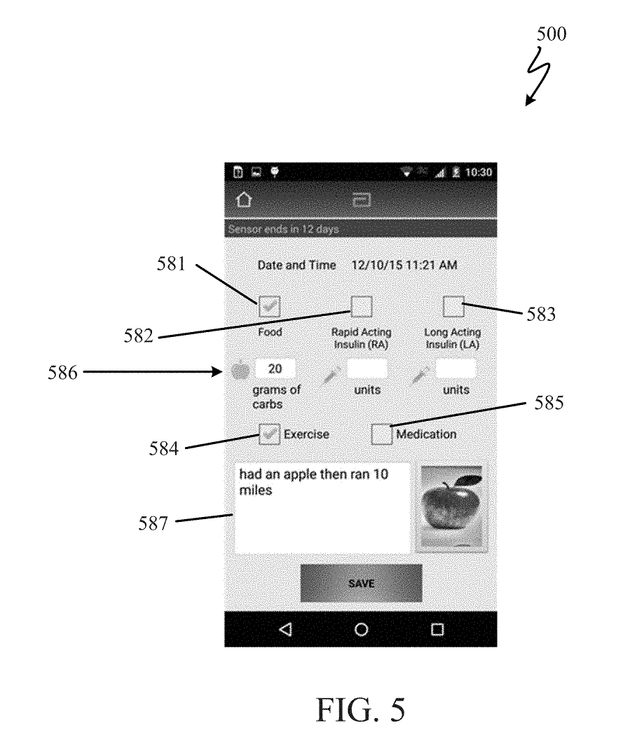

Notes can be used to track behavior such as one or more of food intake, insulin, medication, exercise, sleep, and the like. FIG. 5 depicts an example embodiment of notes entry page 500. Notes can be entered by pressing notes button 458 on result screen 422, the add notes button 280 on home page 200, and from data collections on the logbook page accessed by pressing logbook button 259 on home page 200. Notes can be added by scanning a bar code or chip, or the like, such as of a food item. Notes entry page 500 can include one or more tick fields to characterize the note as conveying information about food intake (field 581), one or more types of medication such as rapid acting insulin (field 582), long acting insulin (field 583), exercise (field 584), and medication (field 585), and the like. The tick fields shown here include an empty box where a user's touch will change the field's value back-and-forth between a yes value where a check mark is present and a no value where the check mark is absent (empty box). Other alternative fields can be used as well, including tick fields with more than two values, and drop down menus.

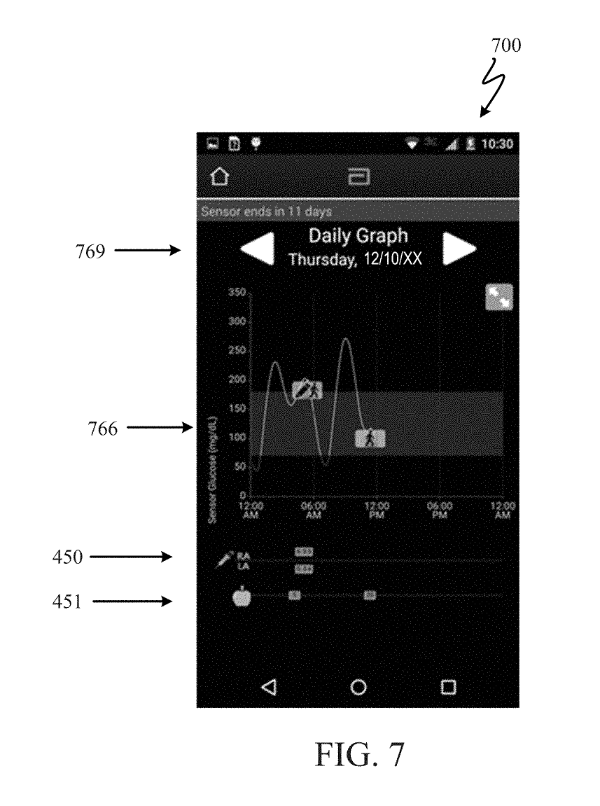

More specific information about quantities of food and insulin can be entered into entry boxes 586 located below corresponding tick fields. At least one freeform text box 587 can be present for entry of a textual note about any action taken by the user (e.g., quantity of time exercised, dosage of a particular medication, type of food consumed) or other condition that the user wishes to record. Text can be entered through a keyboard (or virtual keyboard on a touch screen) or verbally, e.g., by using a speech recognition function. Once notes are entered, they can be saved and one or more identifiers, such as icons representing the added notes, are placed at the appropriate times on various glucose level graphs. For example, a graphical meal icon representing food intake or a graphical medication icon indicating insulin injections can be added to the daily graph 766 (see FIG. 7) or the glucose graph 457 along with amounts or doses taken.

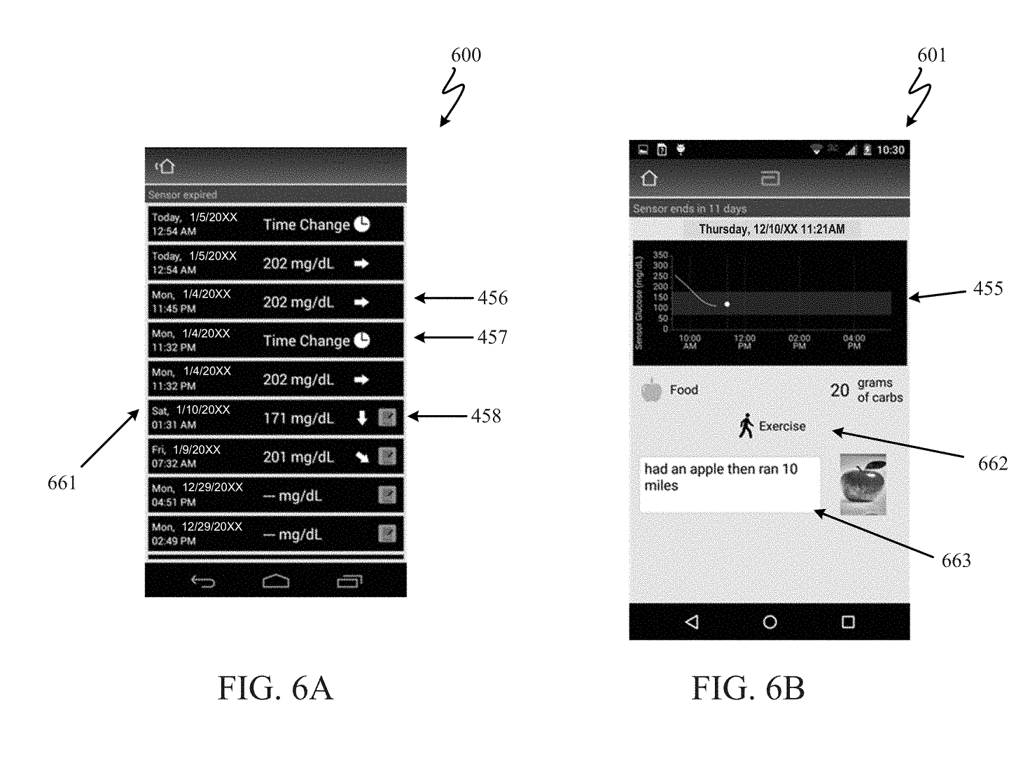

Referring again to FIG. 2, pressing logbook button 259 can open the logbook screen 600, an example embodiment of which is depicted in FIG. 6A. The logbook screen can include entries 661 for each data collection from sensor control device 102 and added notes. Date and time of each entry can be included, along with one or more of a display of a current glucose level, directional rate of change arrows 456, and symbols for time changes 457 and notes 458. Selecting an entry can open a summary screen 601, an example embodiment of which is depicted in FIG. 6B, with detailed information associated with the selected entry including one or more of a graph 455 of historical glucose levels around the time of the log book entry, directional rate of change arrows 456, and any notes 662, free form text 663, and/or images added by the user.

Pressing daily graph button 265, shown in FIG. 2, can open daily graph page 700 shown in FIG. 7. Daily graph page 700 can include a graph of glucose level readings 766 for the day 769 shown at the top of the page and/or timeline graphs 450 and 451 associated with user-entered notes, for example, food intake and insulin usage, identified with symbols. Icons 768 representing, for example, times of exercise, medication dosing, etc., also can be shown on the glucose trace itself within graph 766. The graphs can share the same horizontal time scale axis which can provide a graphical representation of how insulin levels may be affected by exercise or taking food, insulin, or medications. The time scale of the graphs can be changed by using two fingers to pinch or expand the graphs and can be shifted by dragging the graph left or right. Time increments on the graphs can be adjusted, for example, from 30 minutes to 9 hours, allowing either a detailed view, e.g., over a few hours, or an overview, e.g., of about a day and a half. Selecting any of the displayed symbols, for example, medication dosage, exercise, food intake, or insulin usage, can open a pop-up screen displaying notes for that time of day entry.

Daily graph 766 of glucose level readings is similar to the glucose graph 457 of FIG. 4B. Daily graph 766 can display glucose level readings up to a predetermined level, for example, 350 mg/dl. Readings greater than the predetermined level can be sticky, in that they can be displayed as the predetermined level. Gaps can appear in graph 766 if data from sensor control device 102 has not been collected frequently enough, for example, at least once every 8 hours. If the time on reader device 120 changes, a clock symbol can appear, and gaps, overlaps, or hidden data can result.

As described earlier, adding notes can be useful in determining patterns in glucose levels. However, it can be problematic for a patient to remember to add notes, especially after an extended period of time has elapsed since the event. Additionally, examining the logbooks in an attempt to correlate specific activities in the notes with glucose levels can be daunting for both the patient and HCP.

In some embodiments, episodes can be detected by the EIS when glucose level readings are received by reader device 120. Adding notes and other information when specific episodes occur can greatly improve the ability to correlate specific behaviors with corresponding changes in glucose levels. The EIS can prompt the patient to input information when an episode is detected and the patient's responses to the prompt, and other patient-entered notes, can be used to assess the behaviors that may be causing the changes in glucose level.

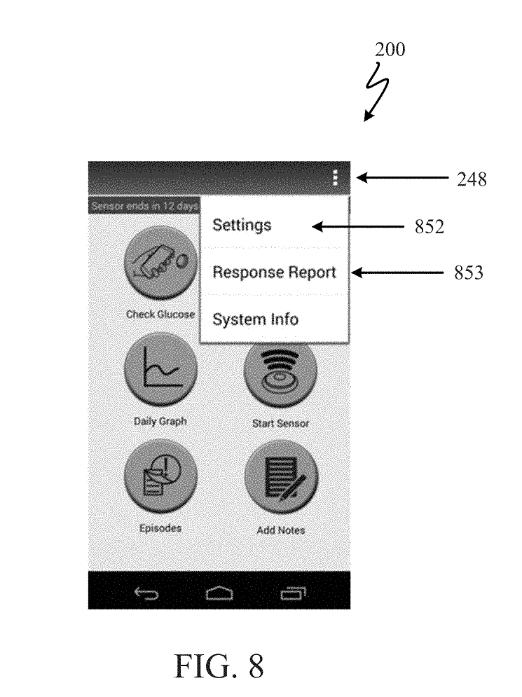

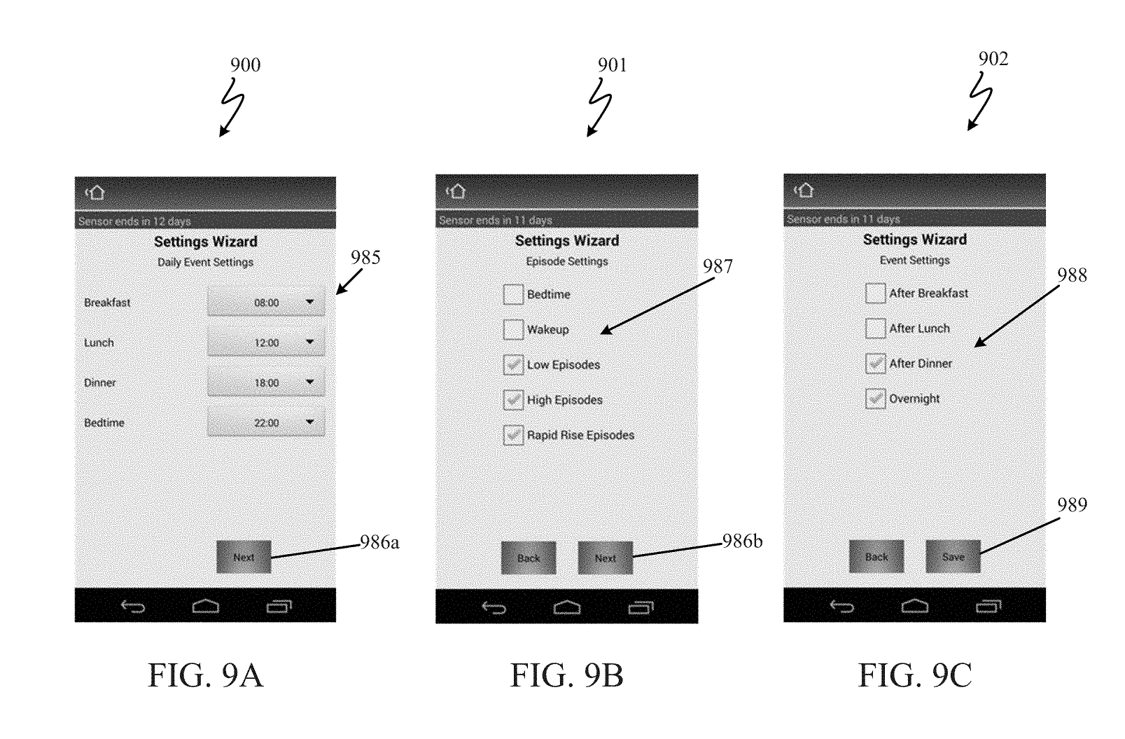

Episode detection can be configured through server 130 or the user interface of the EIS. The process can be similar for both. On reader device 120, menu button 248 can be selected from home page 200 (see FIG. 2) of the EIS. From the dropdown menu shown in FIG. 8, selecting the settings button 852 can start a settings wizard and open the daily events settings screen 900 in FIG. 9A. The daily events can include one or more of breakfast, lunch, dinner, bedtime, and other events, and can be displayed along with default times of day for each of these events on corresponding time of day buttons 985. Selecting the time of day buttons 985 can allow the user (e.g., the HCP, patient, and/or caregiver) to adjust the times to those typical for the diabetic and can set time of day periods for use in certain displays described below.

Once the times of day are set, selecting the next button 986a can open the episode settings screen 901, an example embodiment of which is depicted in FIG. 9B. On screen 901 the type of episodes to be detected can be chosen by selecting check boxes 987 corresponding to the type of episode desired. Episodes can include one or more of bedtime, wakeup, and actual or impending low episodes (low glucose levels, e.g., less than a predetermined value), actual or impending high episodes (high glucose levels, e.g., greater than a predetermined value), and actual or impending rapid rise episodes (e.g., glucose levels are rising at a rate greater than a predetermined rate, e.g., 1 mg/dl or 2 mg/dl per minute), and actual or impending rapid fall episodes (e.g., glucose levels are decreasing at a rate greater than a predetermined rate, e.g., 1 mg/dl or 2 mg/dl per minute). The episode triggers, e.g., the levels and rates of change, can be selectable from predetermined options or can be customized. Additional episodes can be defined by the user in the EIS.

Once the episode types are chosen, selecting the next button 986b displays the event settings screen 902, an example embodiment of which is depicted in FIG. 9C. On screen 902, the user can choose the times of day that episode detection can occur by selecting fields 988 (e.g., check boxes) corresponding to the times of day. Times of day can include one or more of after breakfast, after lunch, after dinner, overnight, and times of day associated with customized events. Selecting the save button 989 can save the configuration and activate episode detection.

Episode detection can be highly configurable and include many options. Episode detection can be enabled for one or more of selected time of day periods, certain days of the week, specific days, weeks, or months, or elapsed time. When episode detection is enabled, data from the selected time of day periods are used for episode detection, however, prior data outside the selected time of day period can be used to determine if and/or when an episode occurred. Episode detection also can include an option to prompt the patient with one or more predetermined questions if an episode is detected. Predetermined questions can be based on the type of episode detected. Additionally, new questions can be added, e.g., by the HCP, caregiver, patient, or the like.

In some embodiments, the EIS can include an optional automatic configuration wizard to facilitate configuration of episode detection that best suits a patient's needs. Automatic configuration can be implemented from either reader device 120 or server 130 and episode detection can be enabled immediately after automatic configuration completes or after review and manual implementation by the HCP, caregiver, and/or patient.

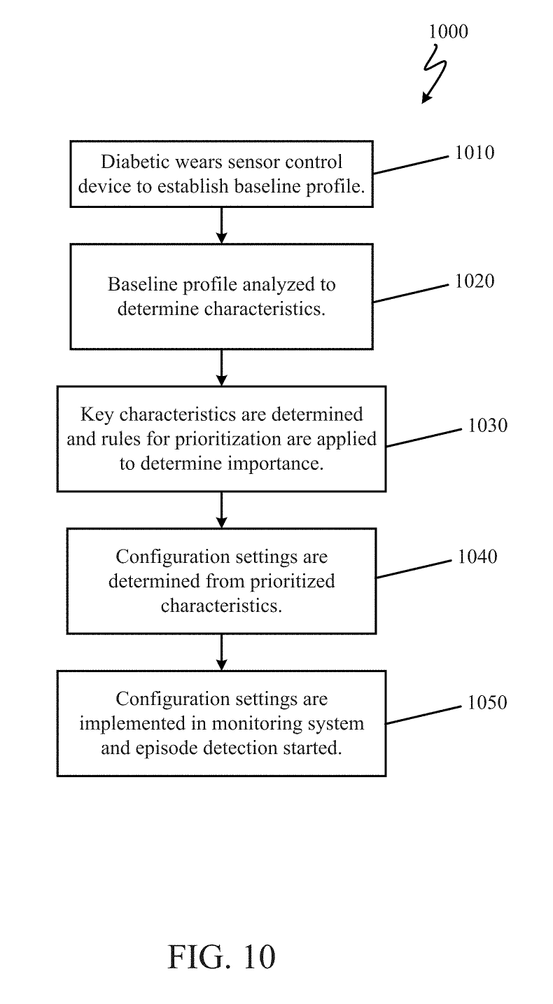

An example embodiment of an optional automatic configuration 1000 is depicted in the flow chart of FIG. 10, which can begin with the patient wearing a sensor control device 102 for a predetermined period of time, for example, 1-2 weeks, to establish a baseline glucose profile at step 1010. In the first instance that the patient wears sensor control device 102 the resulting profile will typically be the first or “baseline” profile for the patient. Of course, the profile can be revised any number of times, such as at follow-up visits with the health care professional, and thus any glucose profile can be used in this method. The EIS can be executed during this time in the masked mode where glucose level readings are not made known to the patient; alternately, this process may be executed with glucose profile data that was collected when reader 120 was unmasked. In some embodiments, the patient can input notes during the automatic configuration period, while in other embodiments, the notes feature is disabled. At the end of the automatic configuration period, the data can be analyzed by the EIS or the glucose monitoring application on reader device 120 using any of the techniques disclosed in the incorporated U.S. Patent Application Publication Nos. 2014/0088393A1 and 2014/0350369A1 to determine characteristics of the baseline glucose profile at step 1020.

In step 1030, key characteristics can be determined and rules for prioritizing them can be applied to determine which are most important for the patient. For example, the EIS can have an automatic time-of-day period enablement function that determines, e.g., based on the user's analyte level profile and/or historical times when episode occurrences are relatively high, whether to enable the EIS to detect one or more types of episodes. In some embodiments, the function can enable the EIS to operate at a first period of time of the day to detect one type of episode (e.g., hypoglycemia, etc.) and operate at a second, different period of time of the day to detect an episode of a second different type (e.g., rapid rise, hyperglycemia, etc.). In other embodiments the function can enable the EIS to operate at certain periods of time to detect episodes of all types. Any combination of the two can also be implemented. If the EIS is enable for a particular time of day period (e.g., 8 am-noon, 6 pm-8 pm, etc.), then the EIS can operate to detect the desired episode types every day of the week during that time period, while the EIS is then disabled at all other times. Further granularity can be implemented such that different time periods are enabled based on the day of the week (e.g., 8 am-noon only every Monday, Wednesday, and Friday, or 6 pm-8 pm only on weekend days). In these and every embodiment herein, the EIS can operate continually or repeatedly in real-time to detect episodes, or can operate only at certain times to detect episodes, as described herein.

In one example embodiment, a prioritization rule or condition set for enabling a particular time-of-day period for low glucose episode detection is:

Enable EIS for a first time-of-day period if:
Likelihood of Low Glucose is HIGH during that first time-of-day period;
and Variability below Median is HIGH or MEDIUM during that first time-of-day period.

Otherwise enable EIS for that first time of day period if (e.g., “Else if”):
Likelihood of Low Glucose is not HIGH for any other time-of-day periods;
and Likelihood of Low Glucose is MEDIUM during that first time-of-day period;
and Variability below Median is HIGH or MEDIUM during that first time-of-day period.

In another example embodiment, a prioritization rule or condition set for enabling a particular time-of-day period for high glucose and rapid rise episode detection is:

Enable EIS for a first time-of-day period if:
Likelihood of Low Glucose is LOW for all other time-of-day periods;
and Median Glucose is HIGH during that first time-of-day period;
and Variability below Median is HIGH or MEDIUM during that first time-of-day period.

Otherwise enable EIS for that first time-of-day period if (e.g., “Else if”):
Likelihood of Low Glucose is LOW for all other time-of-day periods;
and Median Glucose is LOW for all other time-of-day periods;
and Median Glucose is MEDIUM during that first time-of-day period;
and Variability below Median is HIGH or MEDIUM during that first time-of-day period.

Likewise, other combinations of logic can be implemented in a fashion similar to these examples. The determinations of the severity of a risk or characteristic (e.g., low, medium, or high, or others) can be made by reference to the diabetic” s most recent or other selected glucose profile, to a plurality of selected glucose profiles, and/or other historical glucose data. Descriptions of various approaches for defining a particular risk or characteristic (e.g., likelihood of low glucose, median glucose, variability below median) as well as various approaches for quantifying the associated severity of the risk or condition (e.g., low, medium, or high, or others), any of which can be used with the embodiments described herein, are set forth in the following references that are incorporated by reference herein in their entireties for all purposes: U.S. Publ. No. 2014/0187887; U.S. Publ. No. 2014/0188400; and U.S. Publ. No. 2014/0350369.

At step 1040, prioritized characteristics can be used to determine the appropriate settings for configuring episode detection. Configuration settings can be set in the EIS and episode detection can begin at step 1050. Automatic configuration 1000 of episode detection can be instead of, or in addition to, manual configuration. The full range of configuration settings can be accessed by the HCP, caregiver, and/or patient to further customize the configuration after automatic configuration 1000 is complete.

Episode detection, as previously mentioned, can be used with one or more in vivo analyte monitoring systems (both masked and unmasked modes), ex vivo systems, including, for example, ex vivo systems coupled to, including integrated with, an in vivo system. In unmasked analyte monitoring systems, episode detection can occur a) every time glucose level readings are transferred from sensor control device 102 to reader device 120, b) periodically, for instance, every 5 minutes or every 15 minutes, or c) whenever the user activates the EIS or reader device UI. The transfer can be patient-initiated or can be automatically prompted by the EIS.

Automatic prompts to transfer glucose level readings can be configured to occur at predetermined times or intervals, for example, daily at bedtime or at least once every 8 hours.

Glucose level readings measured since the last transfer can be transferred and the episode detector of the EIS on reader device 120 can process the data automatically to detect episodes. If an episode is detected, the EIS can notify the patient that an episode has been detected and prompt the patient to respond to questions about the episode. Patient responses can be time stamped and entered via one or more of a keyboard, a touch screen, a virtual keyboard, for example, a keyboard displayed on a touch screen, an audio recorder, and other input device. Glucose level readings and patient responses can be uploaded to the server 130.

Masked in vivo analyte monitoring systems and ex vivo systems work similarly to unmasked analyte monitoring systems, but can have some differences. For example, in masked systems, to avoid influencing patient behavior, glucose level data may not be displayed. As with unmasked systems, episode detection can occur when glucose level readings are transferred to reader device 120, however, in masked and ex vivo systems, the patient may not be prompted that an episode occurred, but can be prompted to respond to questions, including, for example, questions with respect to activities and conditions at the time of episode occurrence. To further reduce influencing the patient's behavior, episode detection can occur using glucose level readings from a pre-determined period of time, for example, the time period starting 24 hours prior and up to the current time, the time period starting 24 hours prior to and ending 2 hours prior to the current time, etc.

In ex vivo systems, glucose level measurements can be added to the EIS manually, or if the ex vivo system provides communication support, can be wirelessly transmitted to the EIS. Episode detection is performed on the data input into the EIS, and if an episode is detected, the patient is prompted to respond to questions about the episode. Patient responses are time stamped and can be entered via the touch screen and/or via the audio recorder. Glucose level readings and patient responses are uploaded to the server 130. In certain embodiments, an ex-vivo measurement system can be coupled to, including integrated with, an in vivo system. For example, an in vivo housing can include an ex vivo test strip port, and circuitry for both in vivo and ex vivo measurements. In these cases, the episode detection and patient response input features can be added to the integrated system.

Episode detection can also occur in the glucose monitoring application. The episode detector can use an array of glucose level readings and configuration parameters as input. Iterative examination of the glucose level readings can be searched for patterns using the chosen configuration parameters to define criteria for an episode. Episodes can be detected based on the defined criteria and displayed on the result screen. When the episode detector algorithm is active, glucose data preceding the period of interest can be included as input, ensuring accurate episode detection and start time, particularly for an episode that may span the start time of the time period of interest. Additionally, patient responses to questions about the detected episode can be stored on both reader device 120 and the device executing the EIS.

Both the glucose monitoring application and the EIS can operate on the same set of glucose level readings. Running independent episode detection algorithms on both systems can allow configuration of the glucose monitoring application to detect a subset of episodes that the EIS detects, or stated alternatively, the EIS can be configured to detect a superset of episodes that the glucose monitoring application detects. The most clinically meaningful subset of episodes for patient responses can be identified without overwhelming the patient to respond to every episode that can be detected. The EIS can detect all episodes, and as the patient becomes better able to control glucose levels based on behaviors, new episodes can be added to, or replace, the episodes in the subset detected by the glucose monitoring application to further improve patient control of glucose levels.

The glucose monitoring application and the EIS can have different configuration settings, so detected episodes are not transferred from the glucose monitoring application to the EIS. In some embodiments, one or more of the glucose data and patient responses, along with the time stamp, are transferred. The EIS can execute the episode detection algorithm and match patient responses to detected episodes based on the time stamp. Additionally, the EIS can execute routines for identification and characterization of new episodes.

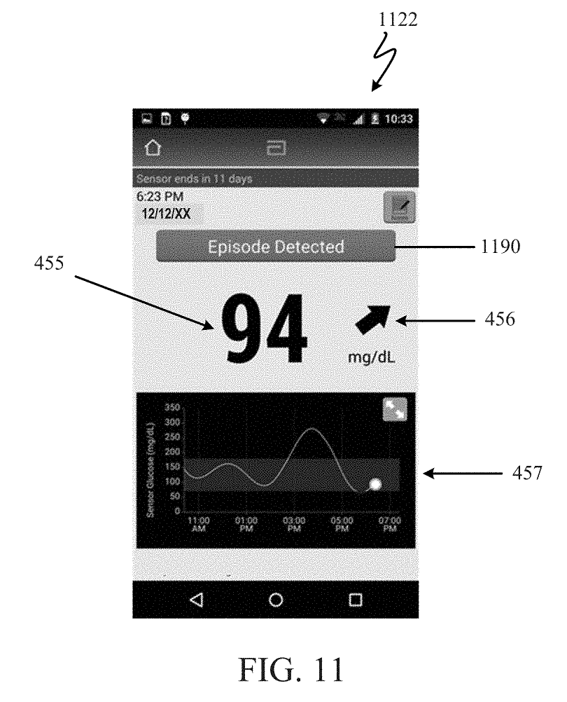

As described previously, when an episode is detected, or multiple episodes, within the previous predetermined time period, for example, the previous 8 hours, the patient can be notified by the EIS and result screen 1122, an example embodiment of which is depicted in FIG. 11, can be displayed. Result screen 1122 is similar to result screen 422 in many respects. In addition to the current glucose reading 455, the screen may include one or more of a trending arrow 456, and glucose graph 457, and an episode detected button 1190. Selecting the episode detected button 1190 can open one or a series of episode response screens displaying questions related to the detected episode. For every episode detected, a separate episode response screen can be displayed, for example, if three episodes are detected then a series of three response screens can be shown and the user can sequentially work through each one.

In an alternative embodiment, a “notification” feature, such as those commonly incorporated into smartphone operating systems, can be used to notify the user of an episode and provide that user with a mechanism to activate the episode response screens. Furthermore, both the episode detection button and the notification feature may be used concurrently.

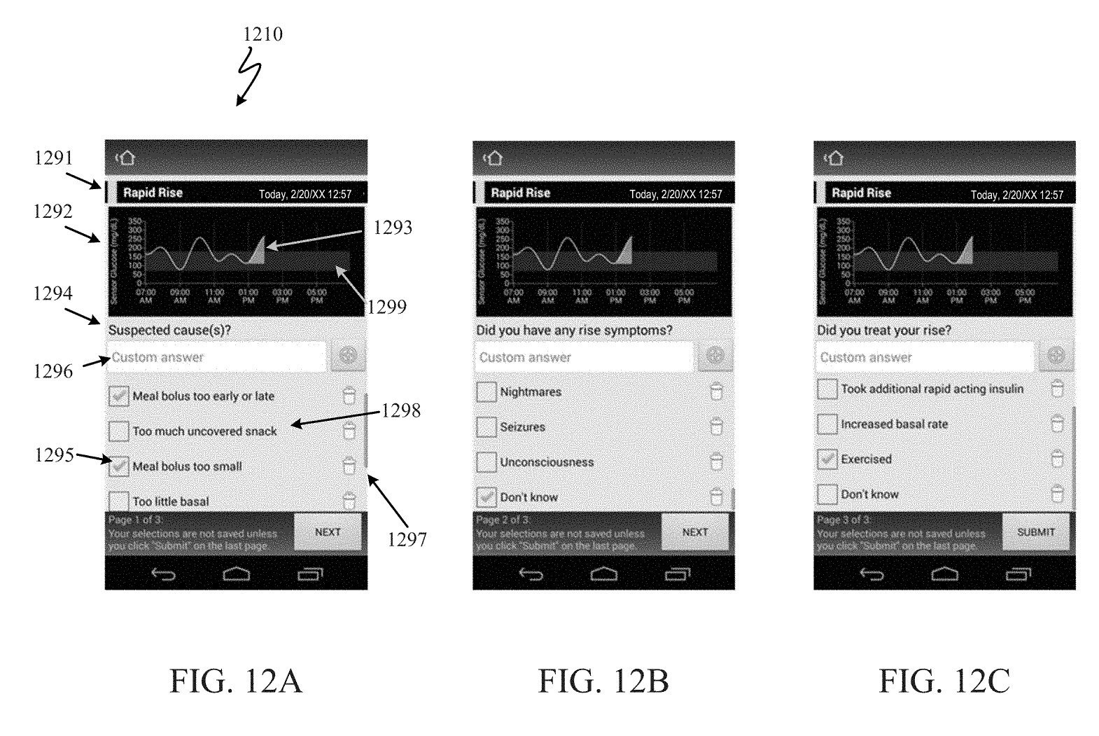

FIG. 12A depicts an example embodiment of an episode response screen 1210. Here, screen 1210 includes an area 1291 where the type of episode detected is displayed. A graph 1292 of recent glucose data, that includes highlights 1293 of the episode, can be displayed. Here the highlight is the shading of the space beneath the graphed curve, and can be in an alternative color from the curve itself. In some embodiments, graph 1292 can be similar to graph 766 described earlier and can also include medication timeline 450 and meal timeline 451 displayed above or beneath graph 1292. The region 1299 of glucose values within the graph that are considered to be normal or within a tolerable range can be denoted, such as with lines or the shading 1299 depicted here.

A question related to the episode can be displayed in area 1294 and a picklist 1298 of possible answers (e.g., reasons or responses), each with a corresponding tick field (e.g., a check box) 1295 for selecting that answer can be included on the response screen 1210. If multiple questions related to the episode are presented, then a picklist 1298 for each question can be included, with each picklist 1298 configured as a drop down menu. In addition to the predetermined responses, a free-form text entry field 1296 can be provided for custom responses (i.e., a response using terms chosen entirely at the user's discretion). If the patient adds a custom response to the text entry field 1296, the custom response can be automatically indicated as an answer, along with any other predetermined response that was selected.

In an alternative embodiment, the user is not provided with the option or ability to create a custom response in the mobile application (and in some embodiments can be prevented in the web-based software as well). However, if desired, an additional field can be provided where detailed information about the episode can be added, such as a free text note. For example, the user can be presented only with the picklist 1298 in FIG. 12A, and after selecting the one or more causes from the picklist 1298 and entering “next,” the user is presented with a subsequent screen containing the freeform text entry field 1296 where the user can provide additional optional details about the potential cause. In many embodiments the user is required to select a preset choice or answer from each of the one or more pick lists presented (e.g., at least one answer for each question presented, where that answer must be selected from the pick list, and where the user does not have the option to add new picklist answers or otherwise customize the pick list except, in some embodiments, through deletion of a pick list answer that is not applicable to the user as described herein).

An improvement in the techniques of managing diabetes (or other medical conditions) can arise from those embodiments that do not provide the option or ability for the user to enter a custom response but rather force the user to select from the preset pick list choices. These embodiments can raise the likelihood that the user thinks critically about which answer is most appropriate, for example, which cause most likely led to the episode occurrence. This can be important because consideration of the potential causes of an episode (or other relevant question about the episode) is often most accurate when that consideration occurs at the same time as the episode occurrence itself (or shortly thereafter). Use of the preset pick list answers provides generalization (avoids excessive specificity) and uniformity to responses and that can assist and simplify the categorization of responses and data analysis at a later stage. Prevention of custom answers can also help prevent a user from providing responses that may not actually respond to the question. For example, a user might, in response to a question about what caused an episode, provide a custom answer that is only a description of what the user was doing at the time the episode occurred, perhaps to save the context for later consideration. While the user can still take such a route by way of the note entry field, forcing the user to respond to a pick list of potential causes can also force consideration of that issue at the present moment, which can be optimal.

In certain embodiments, the user can choose to add the customized answer to the picklist 1298 of predetermined responses, in which case the customized answer will be displayed the next time the same type of episode occurs. In other embodiments, the customized answer is automatically added to the picklist 1298.

Additionally, any responses that are not applicable to the patient can be removed from the picklist 1298 by choosing to delete that response, in this embodiment, by clicking an associated delete button 1297. The deleted response will not be displayed during subsequent instances where that picklist 1298 is shown.

If more responses exist than can fit on the episode response screen 1210, the unseen responses can be seen by scrolling the screen or the unseen responses can be provided on additional screens. After all the questions for an episode are responded to, the next episode detected in the time period that has not been responded to can appear on the result screen 1100. This process of entering responses about the episode can repeated for each episode detected and not previously responded to in the time period. In some embodiments, only the most recent episode can generate a prompt for responses. If responses have already been provided for the most recent episode, the system may not prompt for responses again. Also, the picklists 1298 can be continually modified such that the most frequently selected responses are shown at the top and the most infrequently selected responses are shown at the bottom with the other response is arranged in descending fashion from top to bottom based on frequency.

By allowing customizations of the picklists 1298, the process of requesting and receiving input from a particular user is customized towards that user. The burden, or amount of effort, that the user subjectively considers to be required to record information about the episode is relatively lessened. This, in turn, lowers the user's subjective resistance to inputting information, thus resulting in greater usage by the user, which in turn improves the evaluative mechanisms of the system and can lead to the provision of more robust data and more accurate feedback to the user or HCP. If acted upon, the user's glucose variability can be reduced and quality of life improved.

Several questions can be associated with a particular episode. Each question can be displayed on its own episode response screen. FIGS. 12A-12C are non-limiting examples of episode response screens for a detected episode “Rapid Rise.” FIG. 12A illustrates a first screen with the question 1294 “Suspected cause(s)?” and 2 predetermined responses selected. FIG. 12B illustrates a second screen with the question “Did you have any rise symptoms?” and one predetermined response selected. FIG. 12C illustrates a third screen with the question “Did you treat your rise?” and one predetermined response selected.

Depending on the episode detected, there can be fewer screens and questions or more screens and questions. Questions and predetermined responses can vary depending on the detected episode, but in many embodiments the first or second screen displayed will query the user with regard to causes of the episode, while the other of the first or second screens displayed will query the user with regard to the symptoms associated with the episode, and the third screen will query the user with regards to what treatment, if any, was applied.

After all episodes are responded to and no new episodes are detected, if the time period is associated with bedtime or wake up, time of day questions can be asked. Time of day questions for a time period can be asked once per day.

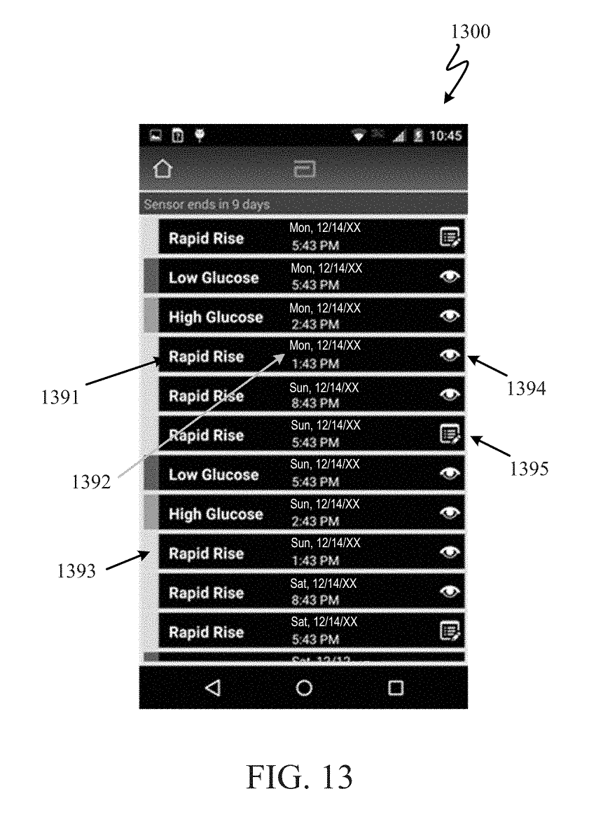

Referring back to FIG. 2, a list of all episodes that have been detected can be displayed by selecting the episodes button 275. FIG. 13 depicts an example embodiment of a list of episodes 1300. One or more of the type of episode 1391, dates and time of episodes 1392, color coded fields 1393 to the type of episode, and icons 1394, 1395 representing whether responses were recorded, can each be displayed. In FIG. 13, a response indicator 1394, for example, an eye-shaped icon, can indicate the patient has recorded responses to questions about the episode, and a non-response indicator 1395, for example a checklist icon, can indicate the patient has not recorded responses. Clicking an episode entry can allow the patient to view previously selected responses to the episode (FIGS. 12A-C) or to allow entry of responses if the patient has not yet responded.

Several reports can be generated to assist the patient, caregiver, and HCP in understanding how the patient's behaviors affect episodes and mitigate the effects of the behaviors. The reports can be generated by reader device 120 using locally stored data or the reports can be generated by server 130 for download to reader device 120 or to a printer.

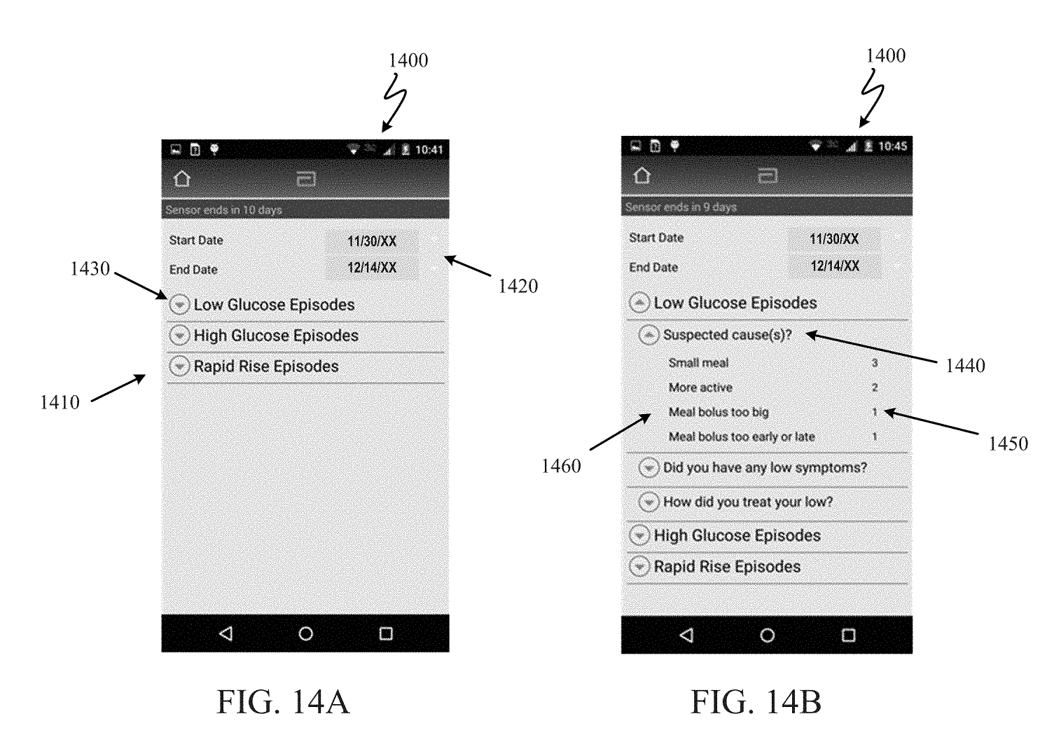

Referring back to FIG. 8, selecting the response report button 853 can open the example response report screen 1400, an example embodiment of which is depicted in FIG. 14A. Types of episodes 1410 having recorded responses can be displayed. The date range for the report can be adjusted using buttons 1420. Clicking a dropdown button 1430 can expand the response report screen, an example embodiment of which is depicted in FIG. 14B, to display one or more of questions 1440 associated with the episode type, the responses 1460 entered by the patient, and a count 1450 of the number of times the response was selected.

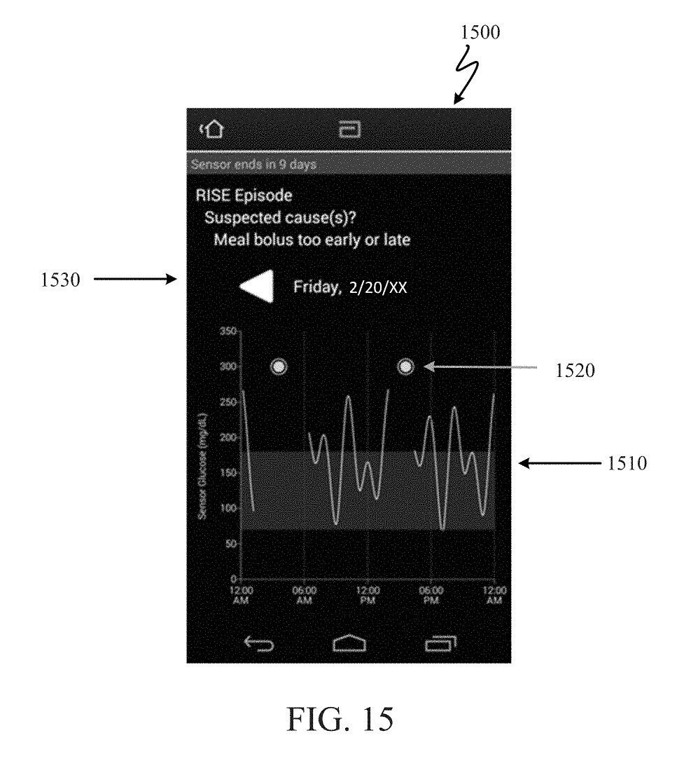

By clicking on a response of interest 1460, for example, “Meal bolus too early or late,” a new screen 1500, an example embodiment of which is depicted in FIG. 15, that can include a glucose level graph 1510 and episode indicators 1520, for example, a colored dot (shown) or vertical line (not shown), can be displayed. The graph 1510 can display the most recent day in which the selected response occurred. If the selected response occurred on additional days, a directional indicator 1530 can appear and the additional days can be viewed by clicking on the indicator 1530.

### Example Embodiments of Web Accessible Episode Investigative Software (EIS)

With respect to computing devices such as local computer 150 or remote computer 160, the EIS will be described as a software package served from a server 130 and accessible by a web browser on the computing device 150 or 160. Mobile devices (e.g., tablets and smartphones) often have web browsers and are considered a subset of such computing devices, although there may be no need to access the EIS via a web browser if the EIS is already installed on the mobile device as an app.

When a sensor control device 102 conveys data indicative of the diabetic's analyte level to reader device 120, that reader device 120 can be programmed to upload the data (either as raw data in the same general form as received from the sensor control device 102, or as algorithmically processed data (e.g., with proprietary data processing algorithms that may apply temperature compensation or other calibration to the data, etc.)) to a database or server 130 over a wireless and/or wired communication path.

After uploading (and any required data processing performed by server system 130 that did not occur already on reader device 120), the data can be stored and associated with that diabetic such that the diabetic, the HCP, and/or a caregiver (or others) can use a web browser presenting the EIS to access the data. In many embodiments the EIS is capable of presenting the data in different report formats that are designed to convey the underlying information in an improved and nuanced fashion that enables the user to readily understand the information and use the EIS as a tool to identify underlying causes of glucose variability.

An administrator, who is a technical person responsible for managing server 130 and/or accounts associated therewith, can provide the URL to use in the web browser to access server 130. The administrator can be responsible for locking and unlocking accounts. There can be multiple types of user accounts, including those associated with the administrator, those associated with an HCP, and those associated with the diabetic or caregiver. Each account type can have a different feature set that is available to the person who is assigned that type of account. An account can be set up by defining a user ID and password, as already discussed to an extent herein. To access the EIS, a user will enter their user ID and password to gain access to their account and the associated features. Users may have multiple different account types.

The administrator is typically a person that has clinical management responsibilities. The administrator's function is to add HCP accounts. In many embodiments, administrators do not have access to personal data or reports of the diabetic or patient. An HCP user can add patient accounts to the system. In many embodiments HCP users have full access to patient data and reports. The HCP users can supply the patients with an initial user ID and password to be used by a patient to access EIS data and reports via a web browser, and to configure the EIS on their reader device 120 to automatically upload data to server 130.

Upon logging in the HCP user can choose a particular patient from a list of patients associated with the HCP's practice. After selection of the patient, the HCP can generate a review session for that patient by selecting an option to generate a new session, typically from the home screen. An example of when this action would be taken is upon the occurrence of a conference or visit between the HCP (or a representative thereof) and the patient.

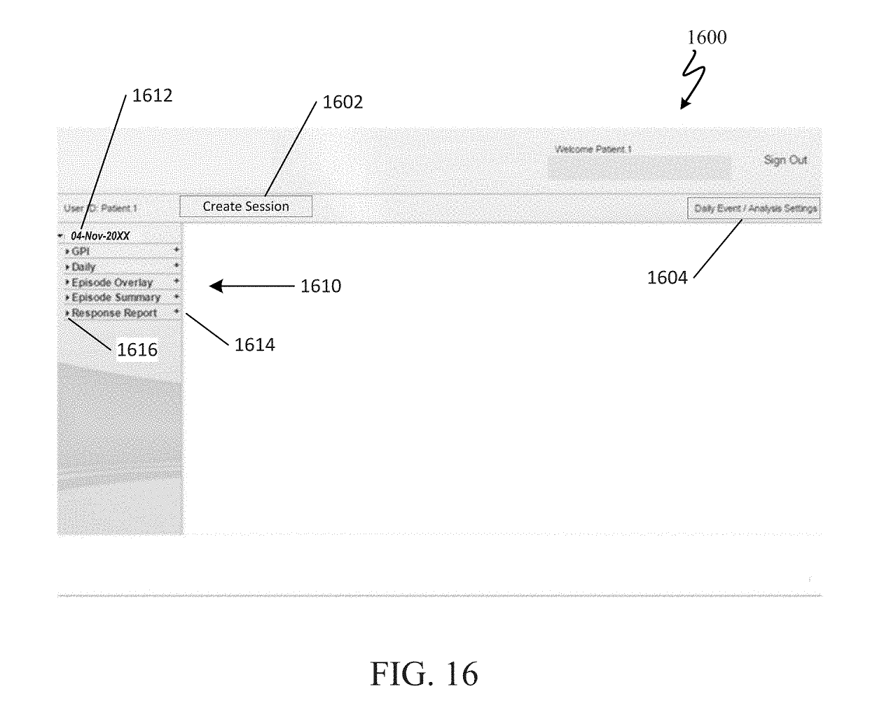

An example embodiment of a home screen or page 1600 that is displayable of the display of the computing device (e.g., 120, 150, 160) via a web browser is depicted in FIG. 16. This home screen 1600 can be displayed to the HCP user upon selection of a particular patient, and can also be displayed to the patient user upon logging in.

On the left side of this home screen 1600 is a navigation bar 1610. A session can be generated by selection of a Create Session button 1602, which in some embodiments can be presented only when new data has been uploaded to server 130 that has not yet been viewed through the EIS or since the date or time when the last session was created. When a new session 1612 is created, its date and/or time can be added to navigation bar 1610 (e.g., 4 Nov. 2015), and reports can be generated for this session using all past data up to that date and/or time.

The user (patient, caregiver, or HCP) can create reports based on data collected and uploaded to server 103. Navigation bar 1610 can provide links to separate report types that can be generated within each session 1612. Whenever a new session 1612 is created, a new set of report links can appear along with the session identifier 1612 at the top of the navigation bar 1610. In the embodiment depicted here, the reports available for creation include one or more of a GLUCOSE PATTERN INSIGHTS (GPI) report, a Daily report, an Episode Overlay report, an Episode Summary report, and a Response report. These various report types will be described in greater detail herein.

Prior sessions 1612 (not shown here) can also be displayed below the current session 1612. Reports generated in those previous sessions 1612 can be viewed and edited, but in many embodiments, are based only on the glucose data that was available up to the day and/or time when that prior session was created.

A new report can be generated by selecting the appropriate field 1614, which in this embodiment is a plus sign (+) next to the respective report title. Once the new report is generated, a link to that report is located in a position associated with that particular session 1612 (e.g., beneath) in navigation bar 1610. To the extent multiple reports of a particular type are generated, a list of those reports can be expanded or collapsed by selecting that field 1616.

Also on home screen 1600 is a selectable field 1604 to modify report settings, which is labeled here as Daily Event/Analysis Settings. For HCP users, a selectable field 1606 to return to a patient list screen can also be provided. Navigation bar 1610, field 1602, field 1604, and field 1606 can be available on all report screens, such as those described below.

Each of the reports generated with the EIS can be based on one or more days of glucose data from the diabetic. A graphical, interactive data selection tool can be presented by the EIS and can assist the user in readily (and rapidly) identifying those days that contain the most relevant data for analysis. Those days can then be individually selected and used for running the report.

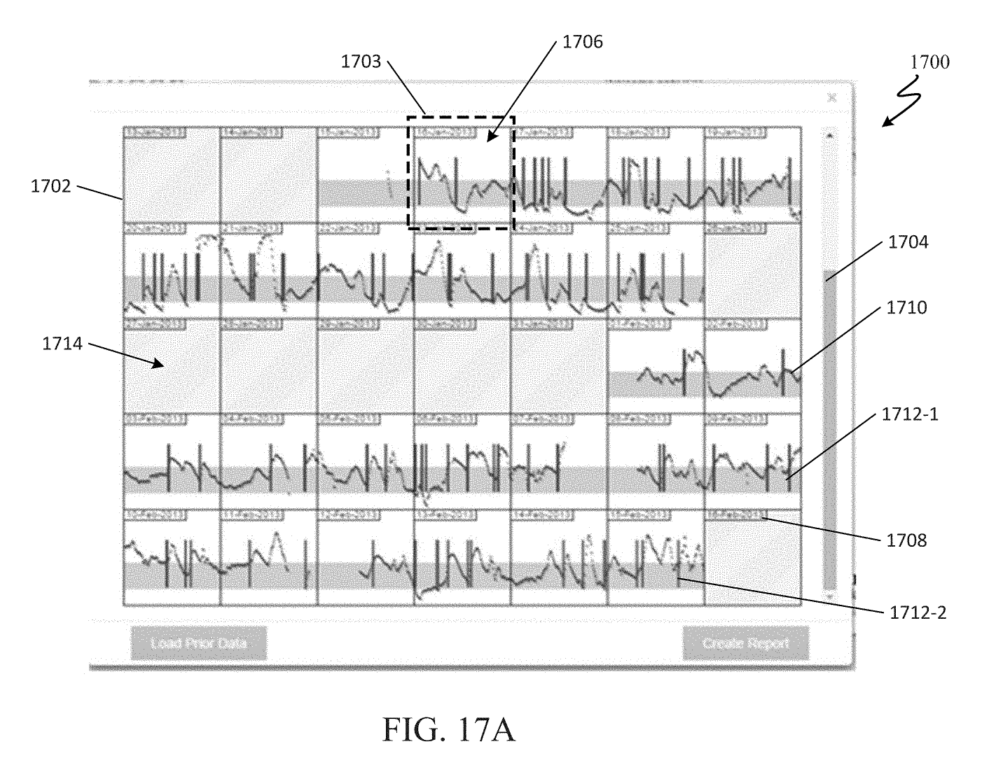

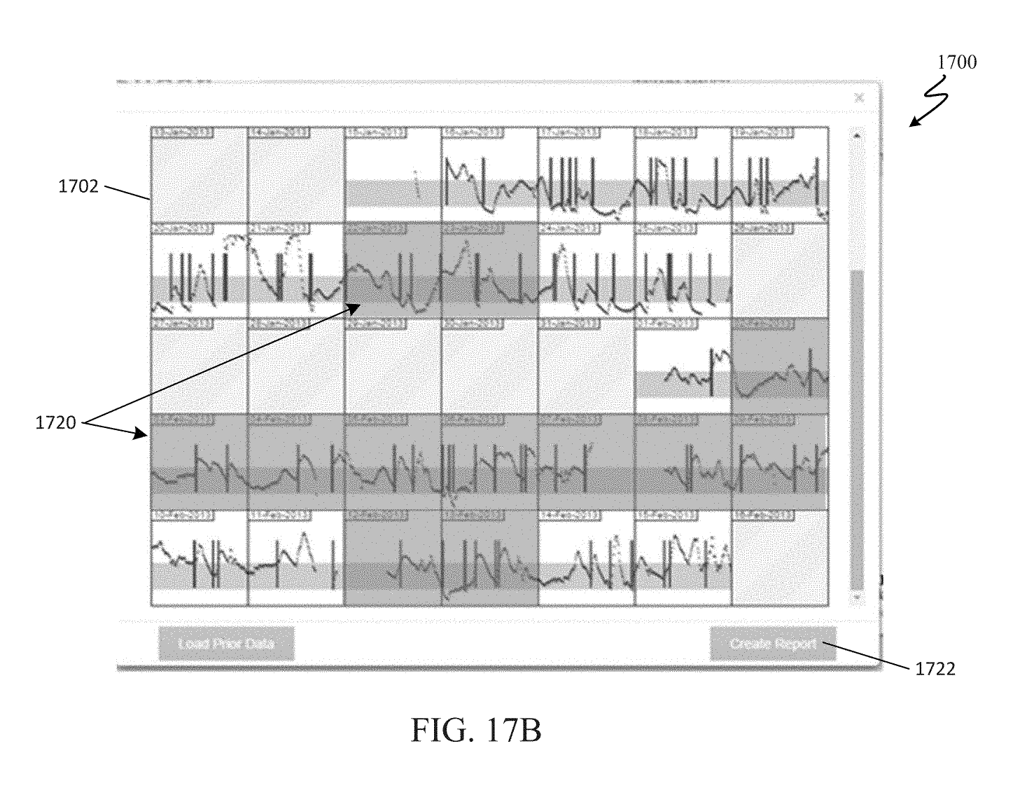

An example embodiment of the data selection tool 1700 is depicted in FIGS. 17A-B. Data selection tool 1700 can be displayed to the user upon selecting the option to generate a report (such as by selecting field 1614 of FIG. 16), or at another point in the report generation process where the selection of the appropriate data set is desired.

In this embodiment, data selection tool 1700 displays data arranged in a grid 1702, where each subdivided region 1703 (e.g., each unit or box), which can be bounded or unbounded, within grid 1702 corresponds to a day, and the days are presented with one week per row (e.g., with Sunday at far left and Saturday at far right). One example of a region 1703 is denoted in FIG. 17A with a surrounding dashed line. The number of days displayed per row is not limited to seven, and any number of one or more days can be displayed per row. Any number of one or more rows can be displayed at any one time (e.g., one, two, three, four, five, six, and so forth.). A vertical scroll bar 1704 can assist the user in scrolling through as many rows as are displayable, the upper limit of which may be set by the amount of weeks in which data is available. For example, there may be five rows of data displayed on the screen at any one time, although ten weeks of data are displayable, with the user being able to scroll through those ten weeks with the vertical scroll bar.

The uppermost row can be the most recent week or current week and the lowermost row can be the week that occurred longest in the past, both as measured from the date and/or time of session 1612, with those rows in between descending back in time in sequential fashion. Alternatively, the weeks can be displayed in reverse fashion, like a typical monthly calendar, where the week that occurred earliest in time is at top and each subsequent lower row is one week later in time.

In alternative embodiments, the grid can be configured such that the days can be displayed vertically in one single column, such that, for example, the most recent day is displayed at top and the day that occurred longest in the past is at the bottom, and vice versa. However, this arrangement does not allow as many of the days to be displayed at any one time.

Within each unit 1703 of the grid is a graphical representation 1706 of the glucose (or other analyte) data for that particular day. A label 1708 that indicates the day of the week and/or the date can also be displayed in or adjacent to unit 1703. The graphical representation, or graph 1706, can have an appearance like that of graph 1292 described with respect to FIGS. 12A-C, or like that of any other glucose graph described herein, including those in the incorporated references. Days for which no data is present, such as day 1714, can be blank, grayed out, or otherwise shaded or indicated to be missing data.

Graph 1706 can include a trace 1710 of the glucose values for that corresponding day, either as a sequence of the measured data points as depicted here or as a broken or continuous line. If each unit 1703 is directly adjacent each other, then trace 1710 can be continuous from one day to the next within each row, as shown here. Each graph 1706 can include an indication or marker 1712 for each episode detected within that corresponding day. The marker 1712 can have any desired shape (e.g., circle, oval, line, curve, square, polygon, triangle, etc.), color, or appearance. For example, marker 1712 can be a vertical line as depicted here, or can be the shaded region 1293 under the trace as depicted in FIG. 12A.

Any combination of shapes, colors, and appearances can be used for marker 1712. For example, episodes of a first type (e.g., high glucose) can be indicated by a first marker 1712-1 of a first shape and/or color, and episodes of a second, different type (e.g., low glucose) can be indicated by a second marker 1712-2 of the same shape but with a different color. In an alternative, one color can be used to indicate episodes of all types, where different episode types are indicated by shape. In still other examples, one shape can be used to indicate episodes of all types, or different shapes can be used to indicate different episode types, but in both cases the severity of the episode is indicated by different colors (e.g., yellow for mild, orange for moderate, and red for severe) or the darkness or opacity of a single color. Severity can also be indicated by the size of marker 1712 (e.g., the more severe the episode, the thicker the vertical line, or the larger the shape, etc.) or by other different shapes or appearances of marker 1712.

The user can then select each day within grid 1702 having data that the user wants to be used to generate the report. As can be seen from FIG. 17A, tool 1700 provides an efficient digest of a large amount of the user's glucose data over what can be a lengthy span of time. This constitutes an improvement that enables the user to more readily and efficiently select data for report generation. For example, because the data for each day is displayed in graphical form, the user may more efficiently identify days with repeating patterns or trends in the glucose traces 1710, and can readily and easily select them. Clusters of days where episodes of a particular type or occurring at a particular time are occurring can be readily identified and selected. Similarly, days where glucose variability and/or episode occurrence was lowest can also be readily identified and selected to search for trends in the diabetic's behavior for those days.

Also, the embodiments of tool 1700 described herein can constitute an improvement in the field of treating diabetes, as tool 1700 allows the identification and selection of the most relevant data sets in a manner that reduces the burden on the user as compared to prior approaches, and which thereby increases the likelihood that the user will use the EIS to investigate and identify the causes of glucose variability and/or episode occurrences, allowing those causes to be ameliorated or treated and thus improving the health and lifestyle of the diabetic or other patient.

Selection of the days can be done directly on the displayed grid 1702, such as by touching each day (in the case of a touchscreen display) or by selecting each day or group of days with a mouse cursor. In either situation, the selection of a particular day can result in only that day being selected (where a previously selected day or days is then unselected).

Selection of multiple days can occur, for example, by holding a first key (e.g., the control key) and selecting each desired day, in which case all selected days will remain selected. A group of days can be selected by holding down a second key (e.g., the shift key) and clicking on a first day of a range of days and a last day of the range, in which case the EIS will automatically select each intervening day in that range. The EIS can be configured so that days 1714 that have no data are not selectable. For reports that rely on only one day of data, such as the Daily report, or on a limited number of days of data, the EIS can be configured to only allow the selection of a number of days equal to or less than the limit.

An example embodiment of a selected group 1720 of days is depicted in FIG. 17B. Here, each day in the selection is indicated by the application of a partially transparent coloring or shading. Other manners of indication can also be used, such as by placing a notable border around the selected days, and the like. As shown here, the user can select non-contiguous ranges of days for use in generating the report. In some embodiments, a default range of days can be selected, such as, e.g., the most recent seven days, the most recent 10 days, the most recent 14 days, etc. When the desired one or more days are selected, the user can generate the report by selecting the Create Report button 1722.

A number of example reports have been and will be described herein. Although these example reports are shown and described in a format that can achieve beneficial results, these reports are not limited to the exact manners in which information is displayed and user interactivity is allowed. Thus, each and every feature (e.g., graph, icon, note, table, list, trace, indicator, selectable field or button, drop down list, response, array of information, ordering of information, etc.) can be modified from the manner in which it is shown and described here while still being within the scope of this disclosure. Further, each and every feature can be added to each and every different report type (or omitted from each and every different report type) unless noted otherwise or logically implausible or impossible, while still being within the scope of this disclosure.

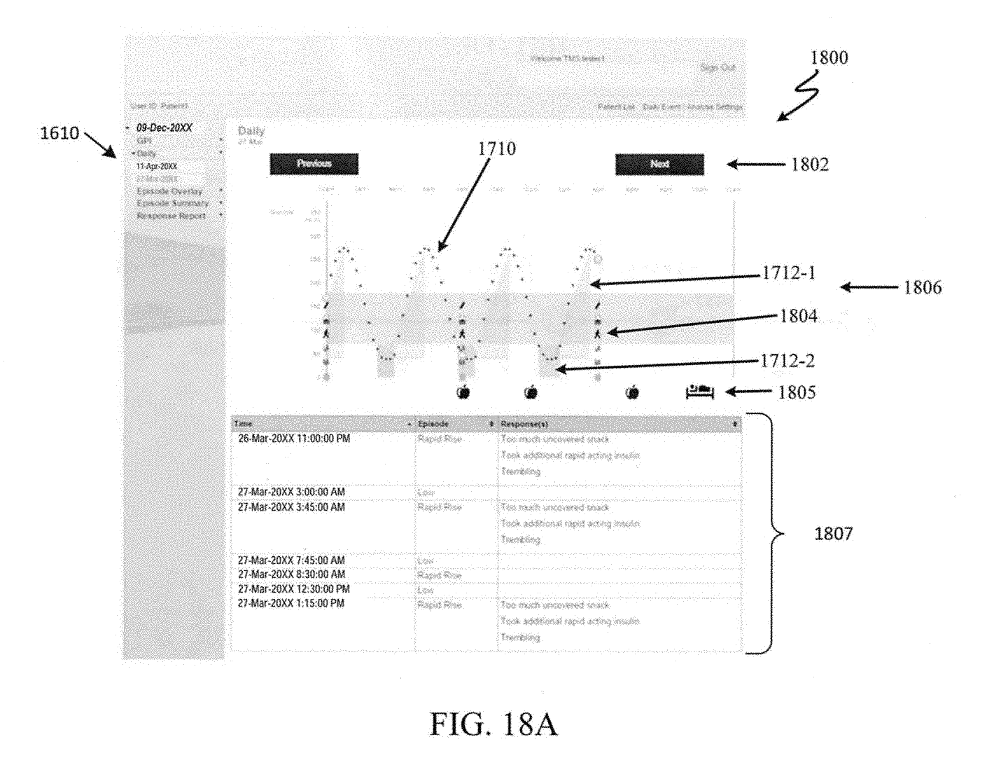

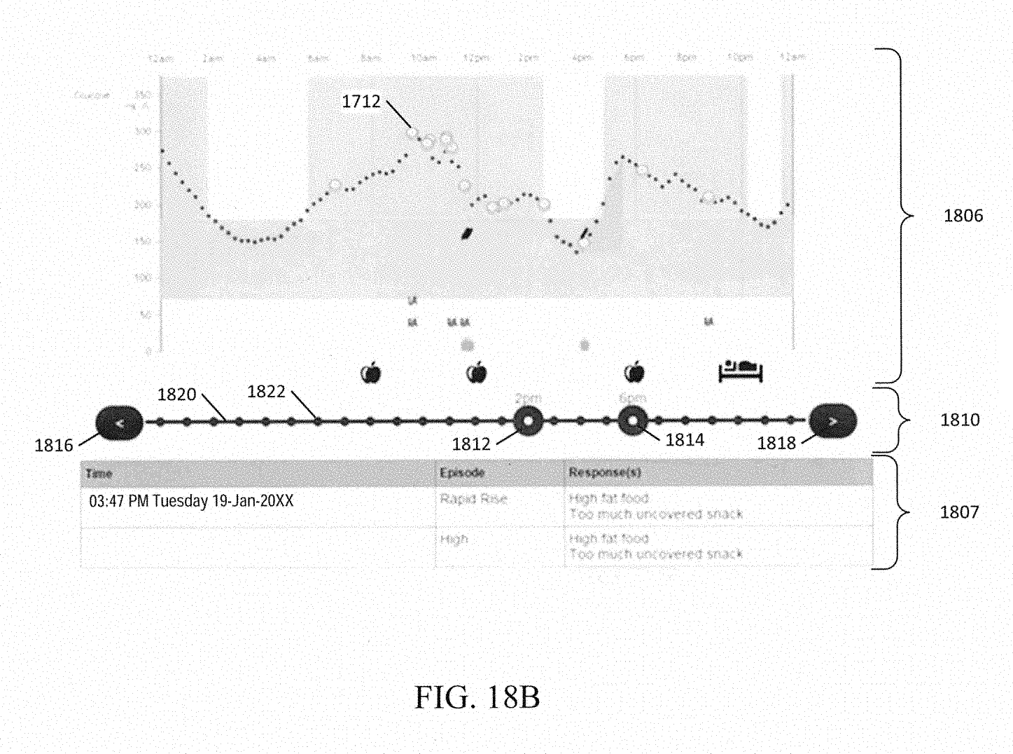

FIG. 18A depicts an example embodiment of a daily report 1800, which, like all reports described herein, can be displayed on the display of a computing device by use of, e.g., a web browser. Daily report 1800 can include a graph 1806 including one or more of a glucose trace 1710 and color-coded markers 1712 for episodes occurring during the time period plotted, typically 24 hours or less. Here, marker 1712-1 is a shaded region beneath trace 1710 that indicates a rapid glucose rise episode (e.g., a rise at a rate that exceeds a threshold for a minimum duration). Marker 1712-2 is a shaded box that indicates a low glucose, or hypoglycemic, episode (e.g., the time in which the user's analyte levels were below a threshold).

Various icons 1804 representing notes entered during that day (such as those described with respect to FIG. 6B) are shown along glucose trace 1710. As described already, each note icon 1804 can indicate one or more of exercise, the consumption of food and/or fluids, the administration of medication (e.g., rapid acting (RA) insulin or long acting (LA) insulin), the presence of a freeform text entry, or the occurrence of sleep, to name a few. Hovering, the cursor over a particular note icon (or touching the note icon as in the case of a touchscreen) can display an overlying pop-up window with additional information about that note (e.g., length of the event, time of the event, calories burned, type of food or drink consumed, amount of carbohydrates or calories consumed, quantity of fluids consumed, dose amount and type, the actual freeform text entry, etc.).

Additional icons 1805 can also be displayed that indicate typical times of event occurrences, which can be set and adjusted by the user. Here, three meal icons (e.g., in the shape of an apple) indicate breakfast, lunch, and dinner. Also shown is a sleep icon (e.g., in the shape of a person in bed) that indicates the typical time the user goes to sleep for the night.

A table 1807 listing and describing each episode can be presented in daily report 1800, for example, beneath the graphical display 1806. Table 1807 can include the time the episode occurred, the type of episode, and any information entered by the user about that episode, such as suspected cause, symptoms, and treatment (see, e.g., the description with respect to FIGS. 12A-C). The table or list can also display note information, for any notes that are entered during the day; it can also display any other recorded information, such as blood glucose readings, etc. Each entry in table 1807 can correspond to a marker 1712 indicating an episode in the graph. Within table 1807, the user can sort episodes and responses by day, episode timestamp, episode type, or response.

Navigation buttons 1802 (labeled here as Previous and Next) allow the user to scroll one day forward or one day back in time, where the EIS can automatically skip those days without data (if any). Selection of one of the buttons 1802 will cause a daily report 1800 to be generated for either the next or the previous day. Each time a new daily report is displayed, it can be added in the appropriate location in navigation bar 1610.

FIG. 18B depicts another embodiment of daily graph 1806 and episode table 1807. In this embodiment, an interactive filter 1810 is located between graph 1806 and table 1807. Filter 1810 allows the user to select which information is displayed in table 1807 based on the position of an indicia for the information in time within graph 1806. Here, the indicia are the episode indicators 1712, and the information displayed in table 1807 is the date and time of each episode, the type of each episode, and the responses recorded with respect to questions posed about the episode. Other information such as notes, photos, etc. can also be displayed. Filter 1810 includes an axis 1820 with two markers 1812 and 1814 that can be slid along the axis by the user in a number of ways. For example, markers 1812 and 1814 can be slid directly, e.g., such as by touching a dragging on a touchscreen or by clicking and dragging with a mouse cursor. The position of marker 1812 along axis 1820 sets the earliest time for the filtering function, while the position of marker 1814 along axis 1820 sets the latest time for the filtering function.

Here, marker 1812 is placed at 2 pm (a label indicating the time at which each marker is placed can be optionally displayed as shown here) while marker 1814 is placed at 6 pm. This placement serves to filter from inclusion in table 1807 all episodes, notes and other information that occurred before 2 pm and after 6 pm in the daily graph 1806. Thus, only those episodes and notes occurring between 2 pm and 6 pm (inclusive or exclusive of those end times) are displayed in table 1807, which in this embodiment is two episodes. Filter 1810 can therefore serve to reduce the amount of information displayed in table 1807, and also to allow the user to focus on only those episodes occurring during a particular time of day. This can be significant for days in which a relatively large number of episodes were detected, such as in the case depicted in FIG. 18B, where approximately fourteen episodes 1712 are indicated in graph 1806.

Markers 1812 and 1814 can also be moved indirectly by selecting fields 1816 and/or 1818. These fields slide markers 1812 and 1814 together to the left (field 1816) or to the right (field 1818) such that both markers 1812 and 1814 maintain a constant spacing (for example, four hours as shown here). Axis 1820 can have multiple discrete landing positions 1822 spaced at even intervals, where the position and movement of markers 1812 and 1814 is limited to only those landing positions 1822. For example, clicking and dragging marker 1812 to the left would cause marker 1812 to jump from one landing position 1822 to the next. Likewise, selecting field 1816 would cause both markers 1812 and 1814 to move one position 1822 to the left, thereby changing their respective times from 2 pm and 6 pm to 1 pm and 5 pm.

In this embodiment, each landing position 1822 is separated by an hour, although a greater or lesser interval can be used. In various different embodiments, the interval between all positions 1822 can be 10 minutes, 15 minutes, 30 minutes, 60 minutes, 90 minutes, and 120 minutes, to name a few. In alternative embodiments, no discrete landing positions 1822 are present and markers 1812 and 1814 can take any position on axis 1820, to simulate a continuous or analog-type movement from the user's perspective. Those embodiments where landing positions 1822 are present can be referred to as having non-analog-type movement.

Filter 1810 can be included with any graph or other representation of data described herein having an associated table, such as table 1807 listing episodes. Filter 1810 is not limited to use with analyte level graphs and episode indicators, but rather can be used to filter based on any type of information presented in the reports, such as exercise indicators, meal indicators, sleep indicators, medication dose indicators (e.g., LA insulin and/or RA insulin), textual notes or comments, or even segments of data display in graph 1806 without any overlying indicator (e.g., the analyte data itself). Furthermore, filter 1810 can be used in any instance, including non-medical applications, where it is desired to focus the display of information in one data structure or visual representation (e.g., table 1807) on one portion or part of another data structure or visual representation (e.g., graph 1806).

In the embodiment of FIG. 18B, filter 1810 is oriented horizontally (e.g., in the direction of the X axis), but other embodiments can utilize a filter 1810 that is oriented vertically (e.g., in the direction of the Y axis), a filter 1810 that is oriented in a third direction normal to the horizontal and vertical directions (e.g., in the direction of a Z axis), and any combination thereof. Furthermore, multiple filters 1810 can be used with the same directional orientation, such as with dual-scaled graphs having, e.g., a left side Y scale representing magnitudes of one type of data and a right side Y scale representing magnitudes of a different type of data. Each filter 1810 can be independently adjusted relative to each other filter 1810, where each adjustment of a filter 1810 can cause a real-time adjustment to the information displayed in the dependent visual representation (e.g., table 1807). Thus, any number of one or more filters 1810 in broad range of different manners.

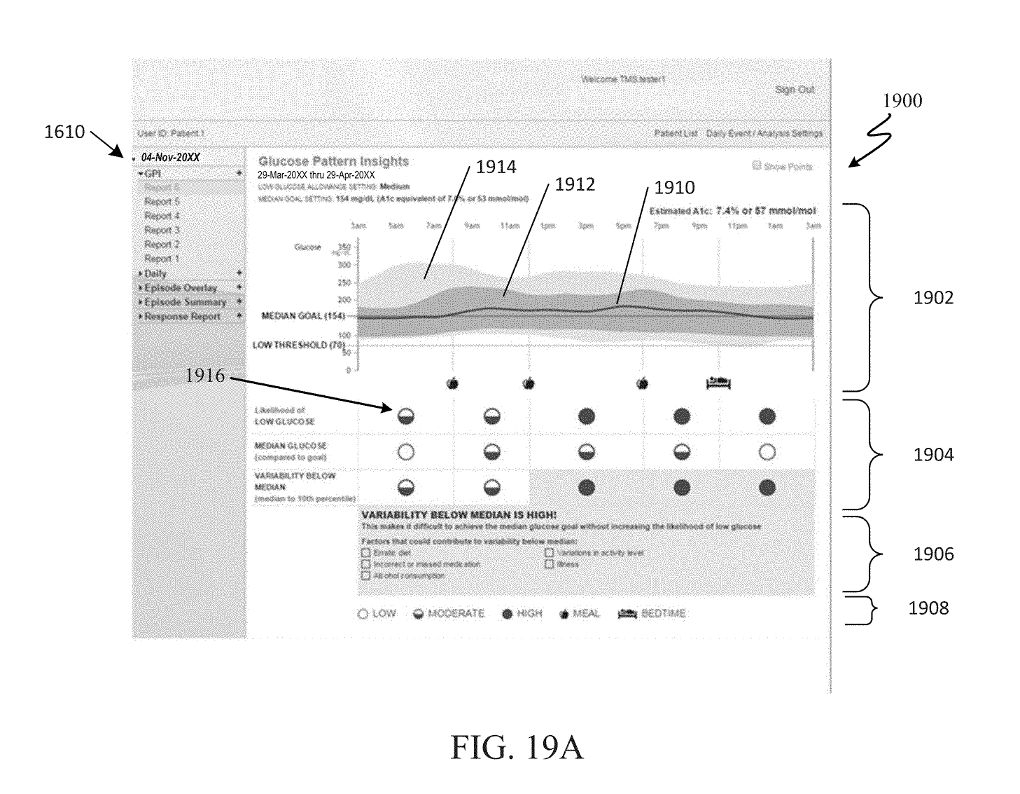

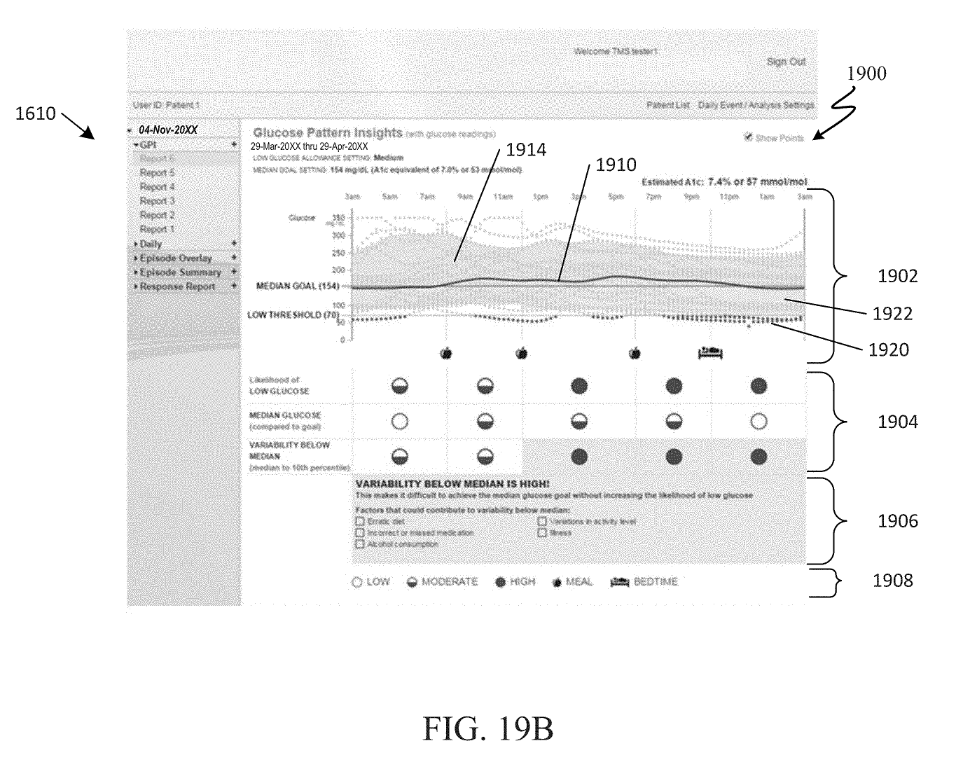

FIGS. 19A-B depict example embodiments of a GPI report 1900. Here, GPI report 1900 includes a first region 1902 that displays an ambulatory glucose profile (AGP) of the user and a second region 1904 that displays assessments of the severity of certain conditions related to the user's diabetes. Examples of GPI reports (referred to as an “Advanced Daily Patterns (ADP)” report) including the AGP are described in detail in US. Publ. No. 2014/0188400, which is incorporated by reference herein in its entirety for all purposes. In light of this incorporated description, the GPI report 1900 is described here in brief.

The AGP region 1902 can include a plot of glucose values for the selected days across a time scale of a typical day. A central tendency (e.g., a median or mean) 1910 is depicted with a trace. Surrounding this trace 1910 is a region 1912 that corresponds to the values of the data that are within a percentile range of the central tendency (e.g., the 25^{th} percentile of data points to the 75^{th} percentile of date points). Next to region 1912 is a second region 1914 that corresponds to the values of data that are within a wider percentile range of the central tendency (e.g., the 10^{th} percentile of data points to the 90^{th} percentile of date points). Regions 1912 and 1914 can be denoted in any desired fashion, such as with different lines, colors, or shadings, to name a few. These regions 1912 and 1914 can be solid or can show each data point as with the embodiment of FIG. 19B. Also shown in the AGP region 1902 is an indication of the user's central tendency goal (here a “median goal”) and the user's low glucose threshold, which can be set and adjusted by the user (e.g., diabetic or HCP).

Region 1904 is a table with icons 1916 identifying the level of severity of the corresponding conditions for subsets of time throughout the typical day. Any number of one or more conditions can be listed and cross-referenced with any number of one or more icons corresponding to one or more periods of time during the day. In this example, there are three conditions listed vertically (e.g., along a Y-axis) that are the: (1) likelihood of low glucose (or hypoglycemia); (2) the median glucose value compared to the median glucose goal; and (3) the variability below the median (e.g., the difference in glucose value between the median and the bottom 10^{th} percentile). For these conditions, a different central tendency, such as mean, can be used instead of the median.

In this example, the day is subdivided into five periods of time, which are not required to have equal lengths, but can be set by the user to correspond to the user's lifestyle (e.g., aligning with meals and bedtime). These periods of time can be aligned (e.g., can use the same time axis) as the AGP 1902 for ease of reference. The icons 1916 in the table can graphically represent the level of severity of the condition during that period of time as indicated by the key in region 1908.

In this example, the diabetic has experienced a variability below the median that is high according to the diabetic's goals set in the EIS, during the period of time from noon to 3 am. There is also a likelihood of low glucose that is high during this time.

Region 1906 can display a notification raising awareness of any one or more conditions that are high and can list potential causes for that high condition. Here, region 1906 displays a notification pertaining to the high variability below the median along with factors that could contribute to that condition.

FIG. 19B depicts another embodiment of GPI report 1900 where the same data of FIG. 19A is used, but depicted in a different fashion. Here, each data point in AGP region 1902 is depicted. Data points 1920 falling below the low threshold can be highlighted (e.g., colored as red). One or more of regions 1912 and 1914 can be omitted (here region 1914 is displayed but region 1912 is omitted). Additional highlighting or indication can be used to denote regions 1922 where one of the tracked conditions of region 1904 was determined to be high. Here, those points associated with a high variability below the median are denoted in the corresponding time range by shaded region 1922.

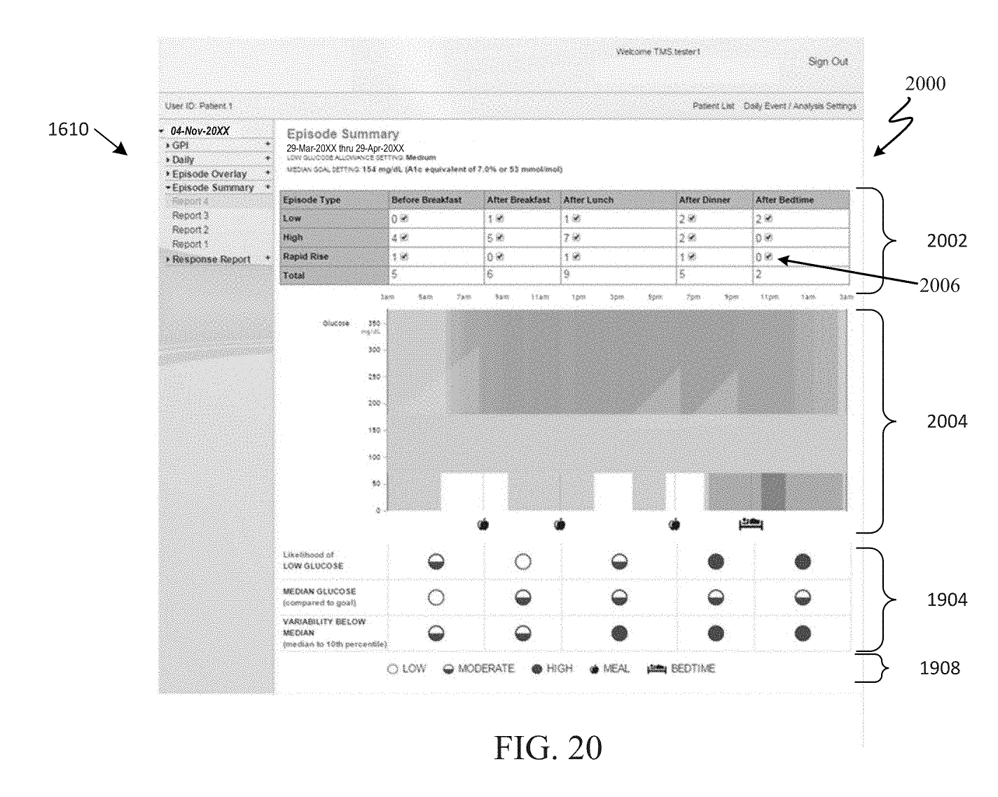

FIG. 20 depicts an example embodiment of an episode summary report 2000, which can assist in identifying episode patterns that may be repeating during a particular time of day. Here, episode summary report 2000 includes a modal day graph 2004 of glucose levels for each selected day over a predetermined period of time, for example, over the entire 24 hours. Also displayed within graph 2004 are markers for the placement and duration of episodes at their time of occurrence with the selected data set.

Episode summary report 2000 can also include a table 2002 that sets forth each episode type (e.g., low glucose, high glucose, rapid rise) along with the number of instances when that episode type occurred during a particular time of day (e.g., which can be the same time periods as depicted in the condition assessment region 1904, both of which can be aligned with the time axis of graph 2004). Total episode occurrences within each time period are also shown. This tabular display 2002 allows the user to readily understand patterns in episode type occurring against particular times of day. A selectable field 2006 (e.g., a checkbox) is present adjacent to each episode count. Selection of this field causes the markers for each episode of that type during that time period to be displayed (with checkbox checked) or hidden (with checkbox unchecked) within graph 2004, which can further assist the user in identifying patterns or times of day where certain episode types are most likely to occur.

In some embodiments of the EIS, an overnight summary report (not shown) can also be provided in a fashion similar to episode summary report 2000. The overnight summary report can differ in that the focus can be for a particular period of time, for example, the overnight period of time, whereas episode summary report 2000 can extend in most embodiments over a greater period of time, for example, over 24 hours. The overnight summary report can include one or more of a table that indicates the number of occurrences of each episode type or risk factor overnight, a plot of glucose level readings, and indicators for episodes and risk factors. Non-limiting examples of overnight low glucose risk factors can include a) bolus within 3 hours of bedtime, b) afternoon or evening physical activity, c) alcohol consumption. Non-limiting examples of overnight high glucose risk factors include a) high carb evening meal or snack, b) high fat evening meal or snack, c) ate out at restaurant.

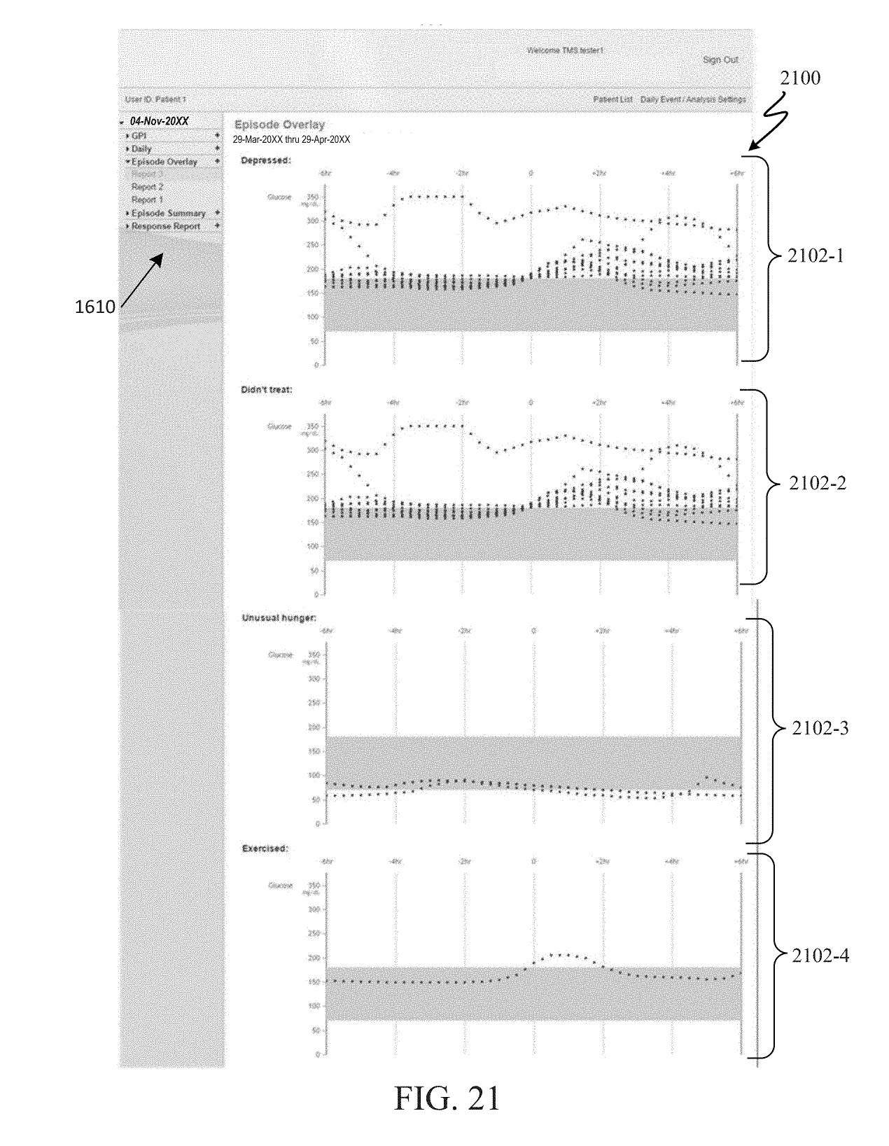

FIG. 21 depicts an example embodiment of an episode overlay report 2100. For each response that the patient identified in response to the questions asked when a particular episode was detected (see the discussion with respect to FIGS. 12A-C), an episode-aligned modal graph or plot 2102 of glucose level readings for every episode having that response identified can be displayed. Time of occurrence of each episode can be aligned at a central axis (e.g., 0 hours) and glucose level readings for a predetermined time period, for example, 1-12 hours, before and after that episode can be graphed or plotted. Episode overlay report 2100 assists in illustrating the effect on the user's glucose levels whenever a particular suspected cause results in an episode.

For example, for each episode detected within the selected date range, the user may have entered a suspected cause. A separate graph 2102 can be generated for each suspected cause, where the graph contains the glucose data associated with each episode where that suspected cause was identified. Although not shown here, graphs 2102 can be similarly generated for each symptom identified and for each treatment identified, or for any other response associated with a question posed about the episode.

In this embodiment, four graphs are shown: graph 2102-1 that includes glucose data for each episode to which the suspected cause “Depressed” was identified by the user; graph 2102-2 that includes glucose data for each episode to which the suspected cause “Didn't treat” was identified by the user; graph 2102-3 that includes glucose data for each episode to which the suspected cause “Unusual hunger” was identified by the user; and graph 2102-4 that includes glucose data for each episode to which the suspected cause “Exercised” was identified by the user (showing only one set of glucose data as only one such episode qualifies). The graphs 2102 can be arranged in sequential fashion where the response with the most qualifying episodes is listed at top and the response with the least number of qualifying episodes is listed at bottom.

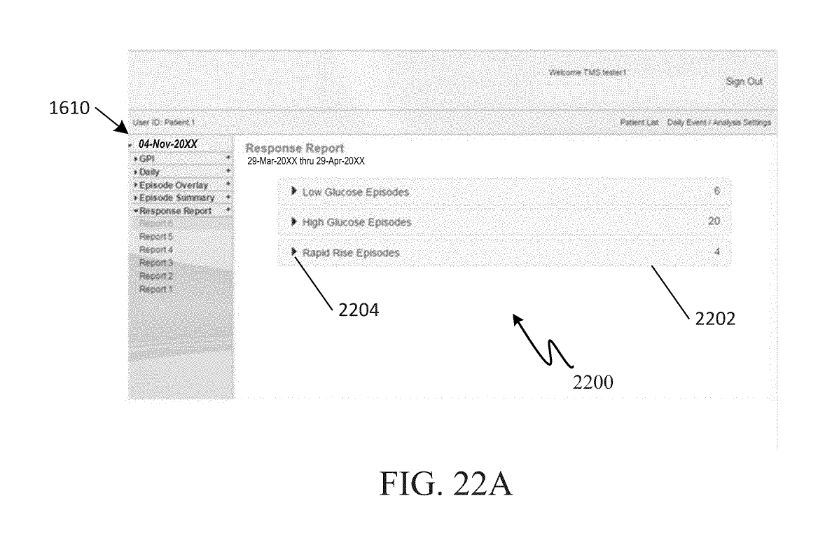

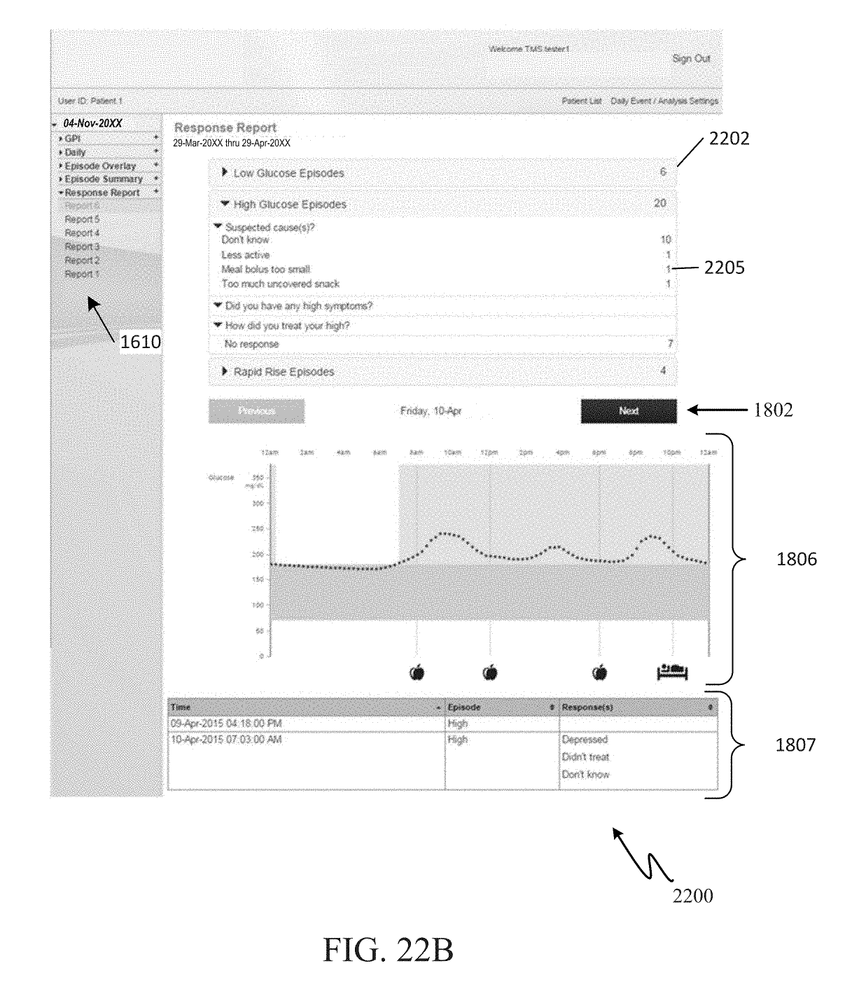

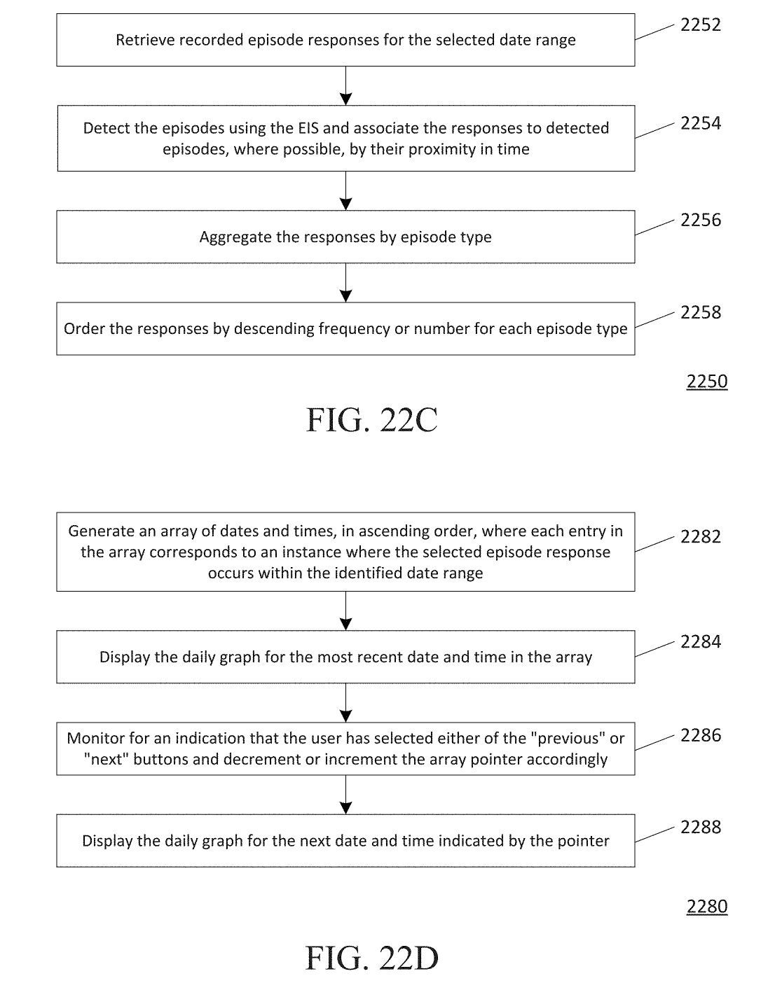

FIG. 22A depicts an example embodiment of a response report 2200, which can assist the user in quickly identifying days of interest—that is, days where episodes have most frequently occurred. This report can assist (e.g., alleviate a perceived need by HCP's) in replacing the practice of searching through day-by-day to find problems; rather this report aggregates the episode responses recorded by the patient and highlights the responses—responses that represent self-care behavior problems that need to be addressed to reduce glucose variability—that occur the most frequently. In the example in the figure, “High Glucose Episodes” are shown to occur the most frequently. After selection of the date range, generation of response report 2200 can cause the display of an expandable summary 2202 of the number of responses recorded for each type of response associated with each type of episode. Selection of the expander field 2204 can cause the list to expand for the associated episode type.

FIG. 22B depicts an example embodiment of response report 2200 after the high glucose episode type has been expanded. As seen here, a list of each question propounded to the diabetic in response to the detection of an episode type is displayed (e.g., those questions described with respect to FIGS. 12A-C). These lists can also be expandable such that expansion causes every response type given to the particular question to be displayed along with the number of instances that particular response was indicated. The responses can be ordered by descending number of instances to facilitate quick identification of the response or problem that occurs the most frequently, which is typically the problem that should be addressed first by the HCP and patient in order to maximize reduction in glucose variability. Selection of a particular response row 2205 can cause the display of one or both of a daily graph 1806 along with an episode table 1807. Other graph types can also be displayed. The displayed daily graph 1806 can be associated with, for example, the most recent day in the selected date range where that response type was actually indicated by the user. Selection of the next or previous buttons 1802 can advance to the next day or backtrack to the previous day where this response was actually indicated or provided by the user.

Report 2200 thus provides a mechanism where users can quickly identify the most frequent response or problem occurrence that needs to be addressed and drill down to (or identify) the particular days where the response or problem occurred. Users can then examine the more detailed information and glucose traces that are presented on an individual day-by-day and episode-by-episode basis and that are associated with this problem in order to understand what needs to be addressed.

FIG. 22C is a flow diagram depicting an example method 2250 of the sequence of instructions, in a server-based version of the EIS, that can be used to develop report 2200. Note that the episode responses and corresponding date and/or time stamps can be uploaded automatically from reader 120 to server 130. In this embodiment, the episodes are detected using the EIS on server 130 instead of with the version of the EIS being executed on reader 120, and the detected episodes are associated to the episode responses by temporal proximity. This can have the advantage of simplifying the design of the EIS stored and executed by reader 120 in that the stored responses do not need to be associated with episodes and the association does not need to be communicated to server 130. (In an alternative embodiment, reader 120 can store and communicate the association between episodes and responses to server 130.)

Referring now to FIG. 22C, at 2252 the server's processing circuitry 131 (see FIG. 1A) retrieves recorded episode responses for the selected date range from the non-transitory memory 132 communicatively coupled with processing circuitry 131. At 2254, processing circuitry 131 can detect the episodes using the EIS and associate the responses to detected episodes, where possible, by their proximity in time (for example, if the first responses recorded after a detected episode are within a minimum threshold time then those responses are associated with that detected episode). At 2256, processing circuitry 131 can aggregate the responses by episode type. Then at 2258, for each episode type, processing circuitry 131 can order the responses by descending frequency or number.

FIG. 22D is a flow diagram depicting an example method 2280 of the sequence of instructions, in the server-based version of the EIS, that can be used to generate the daily graph or report 1806 for a selected episode response. At 2282, processing circuitry 131 can generate an array of dates and times, in ascending order, where each entry in the array corresponds to an instance where the selected episode response occurs within the identified date range. At 2284, processing circuitry 131 can cause the display of daily graph 1806 (e.g., by supplying information for use in rendering the display with daily graph 1806) for the most recent date and time in the array.

At 2286, processing circuitry 131 can monitor for an indication that the user has selected either of the “previous” or “next” buttons 1802 and then decrement or increment the array pointer accordingly. The “previous” button or the “next” button may not be available for selection on the display if the pointer is at the beginning or end of the array, e.g., if there is no date earlier than the date of the information in the presently displayed graph 1806, then the “previous” button will not be available for selection, and if there is no date later than that of the present graph 1806 then the “next” button will not be available. At 2288, processing circuitry 131 can cause the display of the daily graph 1806 for the date and time indicated by the pointer.

If interactive episode filter 1810 is displayed with daily graph 1806, then processing circuitry 131 determines and the positions markers 1812 and 1814 to be on opposite sides of the time of occurrence of the episode, in order to highlight that episode. For instance, left marker 1812 can be located a predetermined time (e.g., one hour) before the time of occurrence of the episode and right marker 1814 can be located the same or a different predetermined time (e.g., three hours) after the start of the episode, adjusted to the nearest clock hour increment.

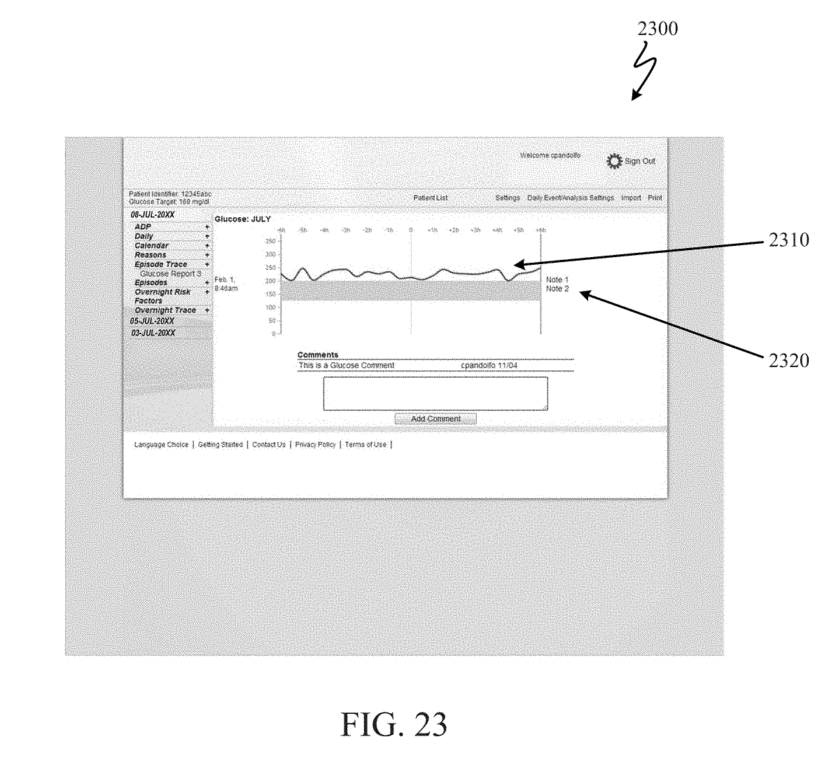

FIG. 23 depicts an example embodiment of an episode trace report 2300. For each episode, a plot 2310 of glucose level readings can be displayed with the episode centered at 0 hours. The plot of glucose levels can span a predetermined time period, for example 6 hours before the episode and 6 hours after the episode. Report 2300 also can display one or more of patient reported episode reasons, symptoms, treatments, and notes 2320 associated with the episode. Report 2300 can allow detailed review of a particular episode.

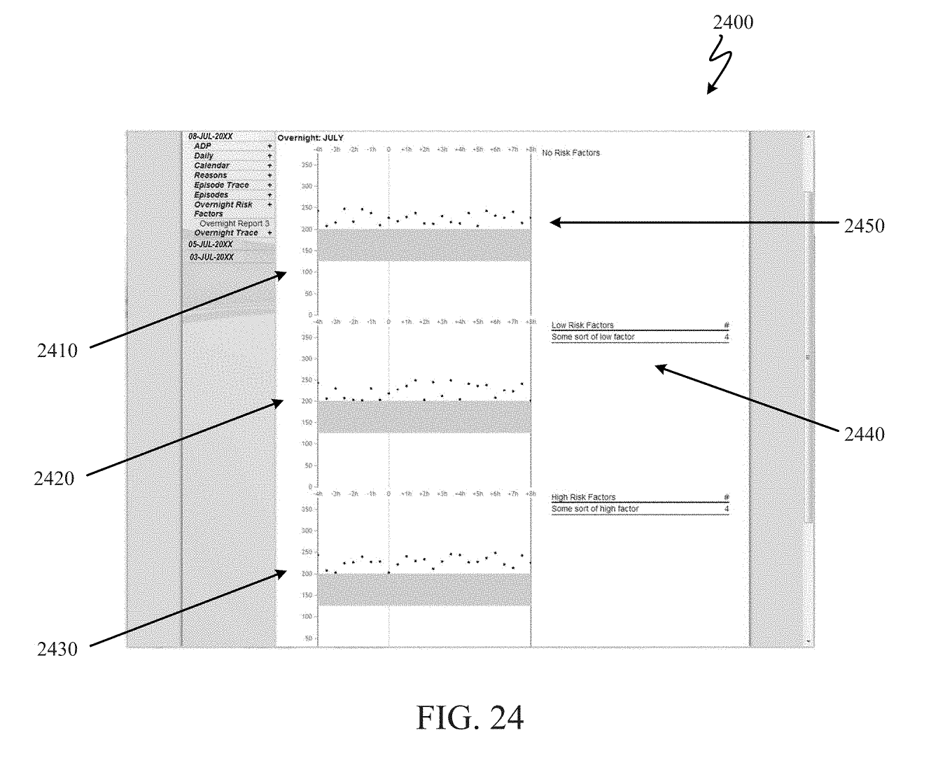

FIG. 24 depicts an example embodiment of an overnight risk factors report 2400, which can include one or more of plots of modal glucose levels 2450 associated with no risk factors 2410, high glucose risk factors 2420, and low glucose risk factors 2430. Each plot of modal glucose levels 2450 can include a tabulation of the number of each type of risk factor 2440.

The reports described herein are merely examples. Embodiments of the EIS can include any combination of some or all of the reports described herein. Embodiments of the EIS can include different reports in addition to, or instead of, those reports described herein. Furthermore, it is stressed that the various fields, data arrangements, icons, notes, markers, and indicators of each graph, table, and/or report described herein can be freely substituted in each and every other embodiment of a graph, table, and/or report described herein, unless stated otherwise or logically implausible.

The local and remote computers 150 and 160 can perform some or all of the functions of reader device 120 and/or network server 130. For example, the local computer 150 can perform episode detection on glucose level readings and prompt the patient for responses to detected episodes. The episode detection algorithm on the local computer 150 can include a configuration to search for all episodes, similar to that of server 130, or a subset of episodes, similar to the glucose monitoring application on reader device 120.

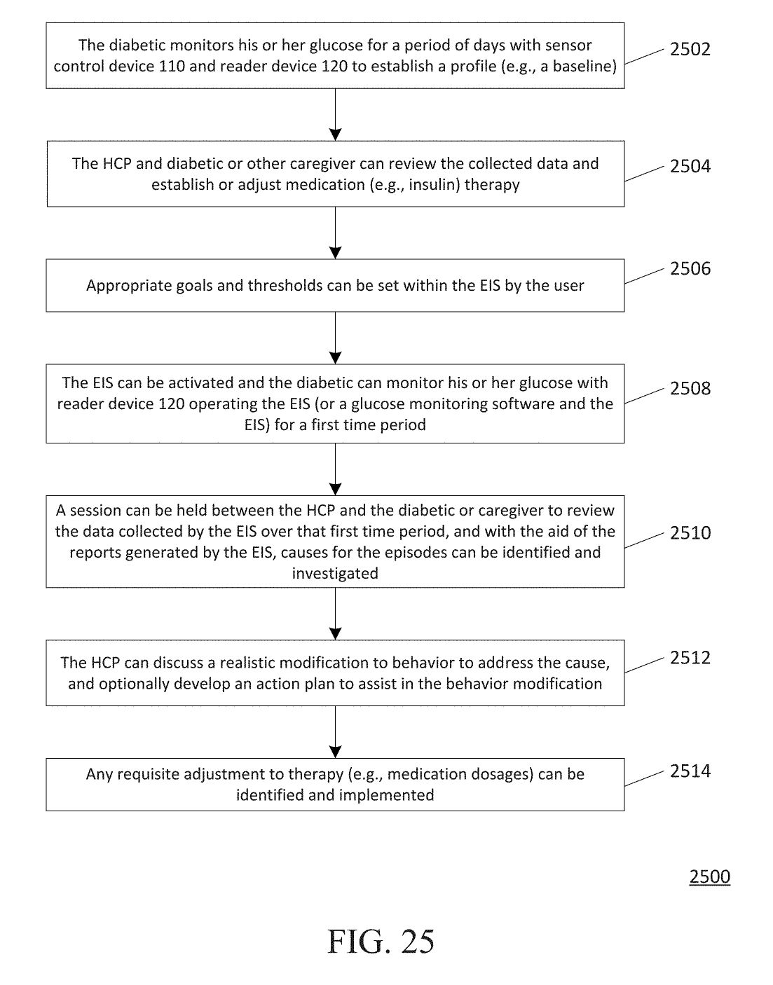

FIG. 25 is a flow chart depicting an example embodiment 2500 of using the EIS. At 2502, the diabetic monitors his or her glucose for a period of days (e.g., 7 days or 14 days) with sensor control device 102 and reader device 120, which can be operating with a masked glucose monitoring software or a masked embodiment of the EIS. This collected data can be used to establish a baseline glucose behavior profile for the diabetic.

At 2504, the HCP and diabetic or other caregiver can review the collected data and establish or adjust medication (e.g., insulin) therapy, such as with the aid of a GPI report 1900. At 2506, appropriate goals and thresholds can be set within the EIS by the user (the HCP, caregiver, or diabetic) to enable the production of the various reports (e.g., a median threshold goal in the GPI report) and to detect episodes and excursions (e.g., low glucose threshold, high glucose threshold, rapid rise threshold, to name a few). At 2508, the EIS can be activated and the diabetic can monitor his or her glucose with reader device 120 operating the EIS (or a glucose monitoring software and the EIS) for a first time period (e.g., a number of days or weeks). The EIS can be set to operate at only certain times of the day, such as those that have shown to be problematic in the baseline data.

At 2510, a session can be held between the HCP and the diabetic or caregiver to review the data collected by the EIS over that first time period. This can include review of one or more reports capable of generation by the EIS. For example, the episode response report 2200 can be generated and the most frequent episode cause or causes can be identified. The daily graphs 1806 within the episode response report 2200 can be then be used to identify the specific underlying behavior causing the episodes. Other reports can be utilized based on the specific situation.

At 2512, the HCP can discuss a realistic modification to behavior to address the cause, and optionally develop an action plan to assist in the behavior modification. At 2514, any requisite adjustment to medication therapy can be identified and implemented, for example, with the assistance of the GPI report 1900. This process can be repeated as necessary until the diabetic's variability levels and/or excursions are reduced to the desired levels.

Example Embodiments of Episode Investigative Software (EIS) with Visit Guide and Action Plan Functionality

In order to reduce glycemic variability, HCPs and patients need to identify and understand the underlying behavioral causes and then take appropriate steps to address them. Embodiments of the EIS are skilled at assisting in the identification of episodic problem categories, e.g., such as too much uncovered snack, but it remains that the HCP and patient need to further understand what specifically is contributing to a particular episodic problem and discuss ways to address the problem to prevent or minimize future episodes.

The traditional process is for HCPs and patients to collaborate and review historical glucose readings and notes, which are often sparse or non-existent, searching for problems but not really understanding how prevalent the problem is. Embodiments of the EIS help the HCP quickly understand the most prevalent problems so the HCP can prioritize his or her time to isolate and better understand the underlying contributing influences on the patient or subject. The EIS provides a mechanism for the HCP and patient to analyze and identify, e.g., “drill down to,” daily traces or trends that illustrate prevalent episodic problems (e.g., high or low excursions, high variability, etc.), and further drill down to specific notes and other details related to the episode. As described herein, example embodiments of the EIS provide tools to make the sub-process of identifying and investigating the most prevalent episodic problems both easier and quicker.

However, in a sizable number of instances the HCP and patient still need to proceed through the additional sub-process of gaining a deeper understanding of the underlying influences and behaviors that contribute to a particular episodic problem, and then collaborate to identify and work out a solution that the patient is willing and able to accomplish and maintain. The entirety of this process does not follow the traditional pattern of a doctor visit, where an examination is performed by the doctor, a diagnosis is arrived at, and a treatment is prescribed. Rather, when using the EIS the process involves diagnosis, investigation into episodic problems with the aid of the EIS, and identification of one or more patient actions to take, and these require a high degree of collaboration between the HCP and patient.

In this context, the EIS is an improvement in the field of diabetes management, as it is a tool that enables the diabetic and HCP to perform a higher level of investigational analysis and take corrective actions not readily achievable in a typical HCP-diabetic relationship. Put differently, the intensive level of human effort required by patients to track or log the contextual information about specific episode types at specific times of day, coupled with the high degree of difficulty in identifying patterns in the patient's glucose data and causes of those patterns that are clinically relevant, which can depend on the quality of the HCP-patient collaborative relationship itself, and/or still further considering the degree of difficulty in causing the patient to take corrective action (e.g., behavioral modification), together constitute a human barrier to furthering the success of treatment. This human barrier is not easily overcome. Use of the EIS is intended to enable the patient and HCP to overcome this barrier. The collection of clinical data demonstrating the efficacy of the EIS in this regard is on-going.

Example embodiments of the EIS can include a visit guide structure and an action plan control interface structure to assist HCPs and patients with this more involved sub-process. Briefly, the visit guide structure aligns collected data based on its temporal relationship to the HCP visit, e.g., whether the data was collected before or after a particular HCP visit. The visit guide structure also provides a step-by-step textual guide, or a script, that sets forth the order of actions that should occur during an ideal visit by the patient with the HCP. The action plan structure delineates the actions agreed to be taken by the patient following each visit in order to address the episodic problems identified. Embodiments of the EIS also provide a mechanism for tracking the degree to which the patient complies with those actions.

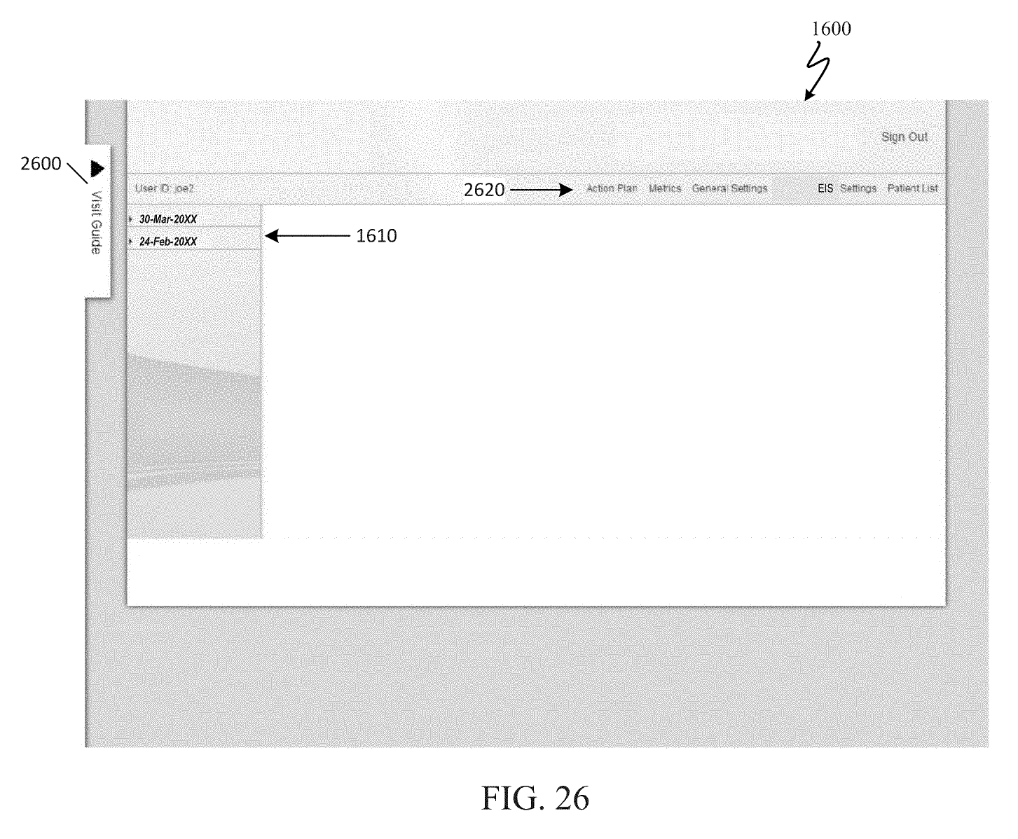

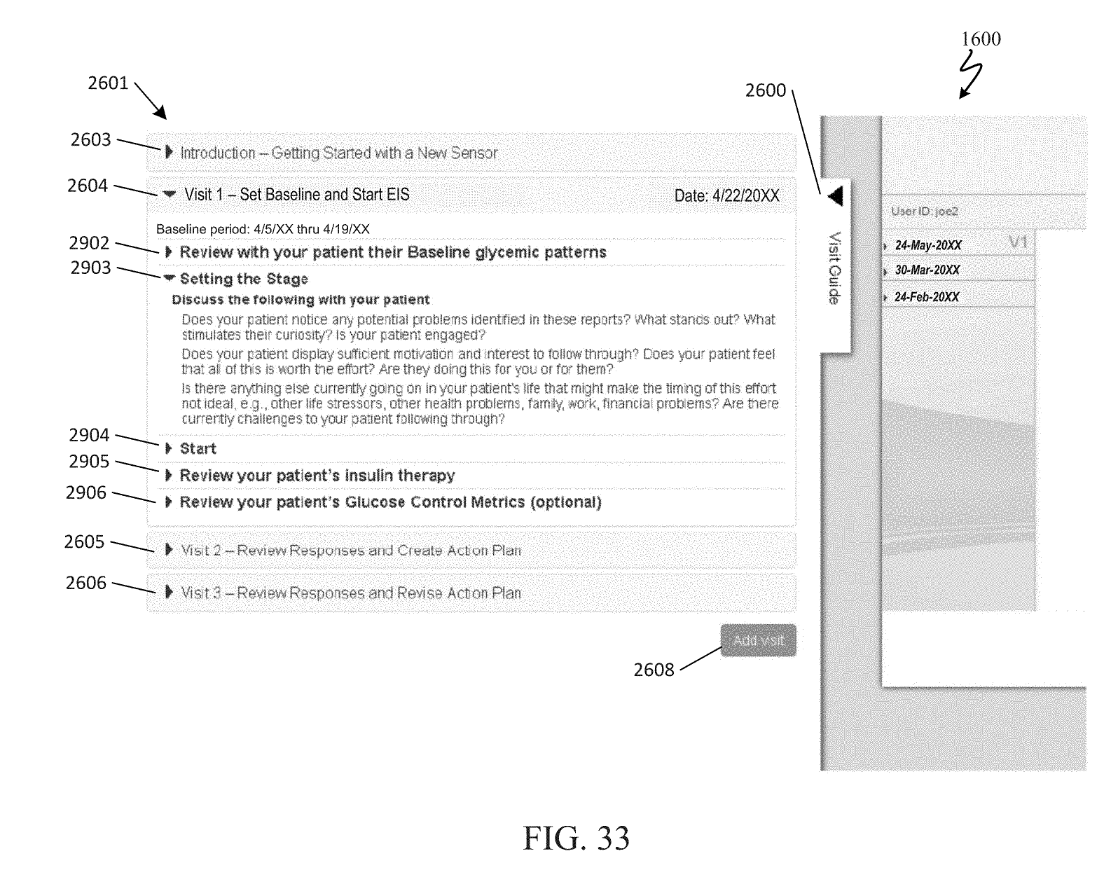

FIGS. 26-56 depict example embodiments of graphical user interface (GUI) screens for the EIS containing visit guide functionality and structure and are displayable on any computing device operating the EIS, such as through a software application resident on the computing device or through an internet browser. FIGS. 26-51A and 52-56 depict example embodiments that are intended for (but not limited to) display on a large screen computing device using an internet browser or web application, referred to herein as the dual access platform since, in many embodiments (but not all), this is the version with greater functionality that the HCP would most often access and that the user can access also.

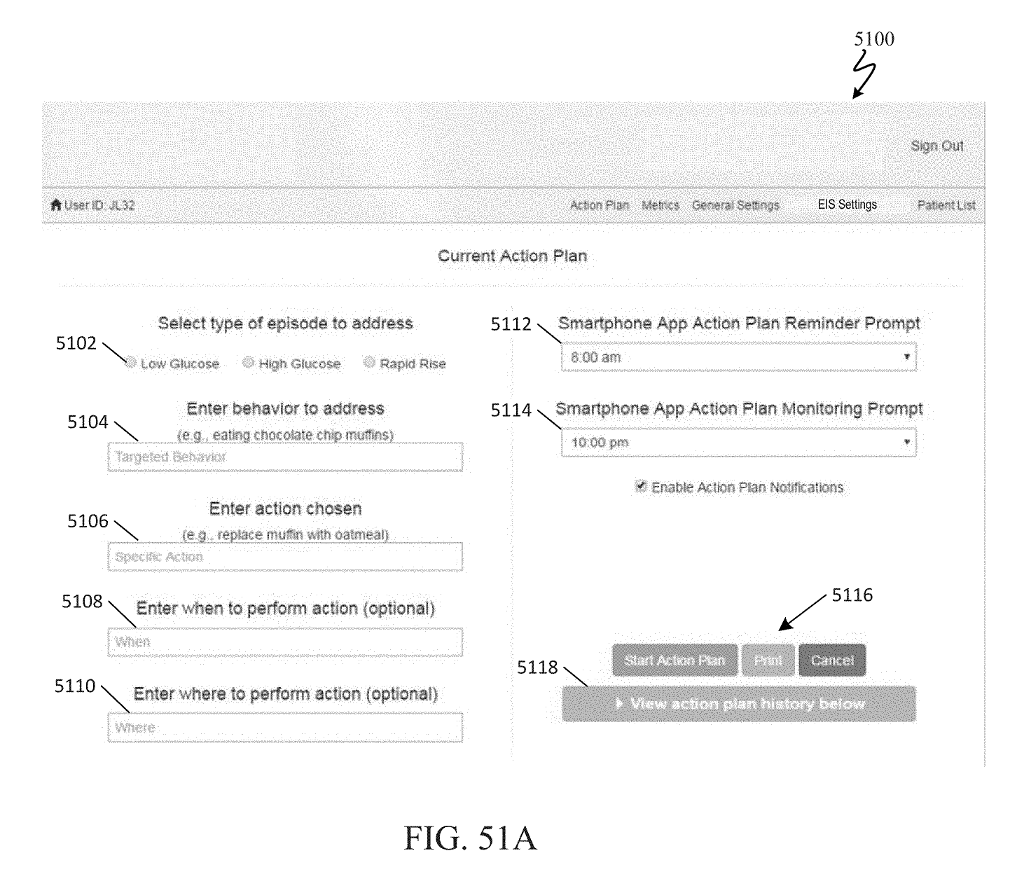

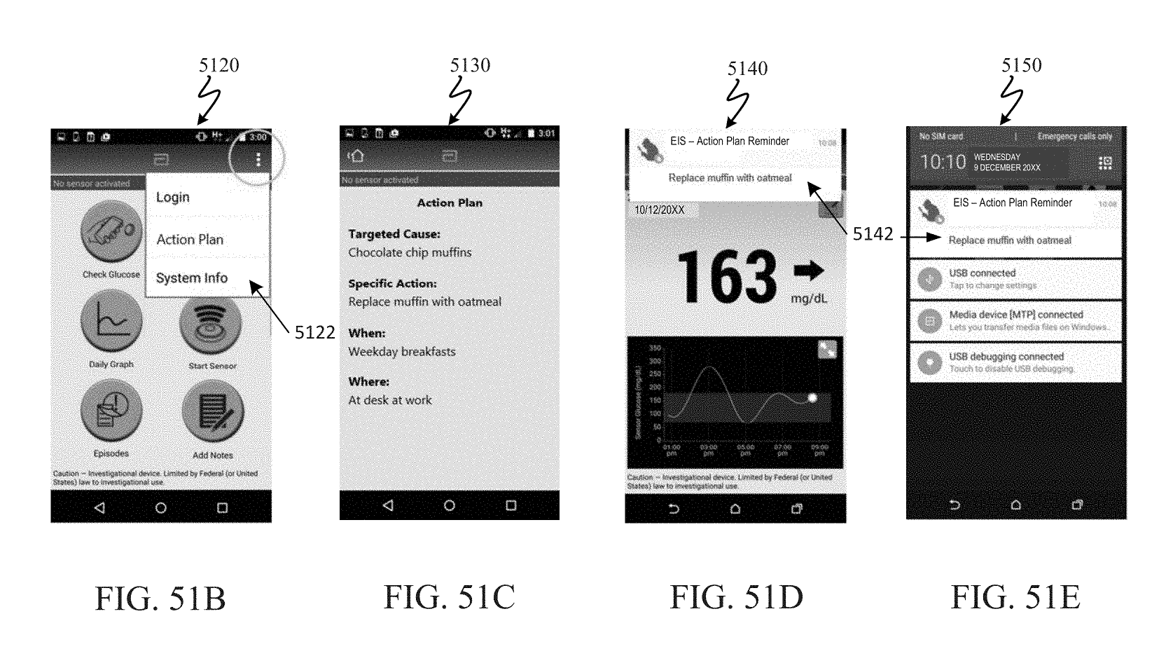

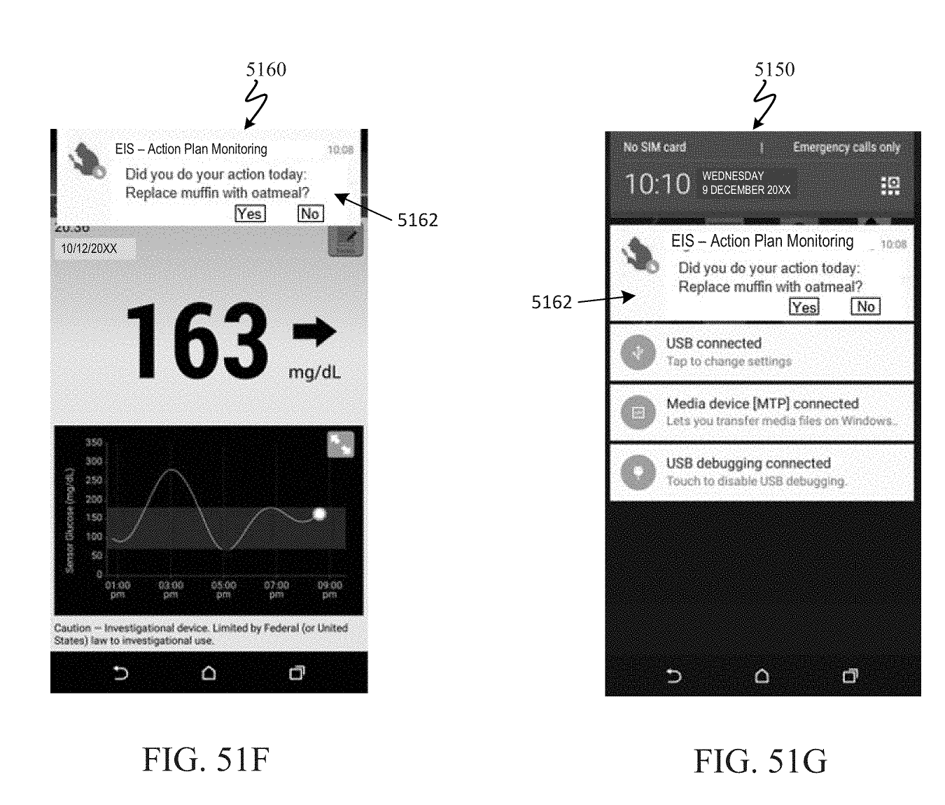

FIGS. 51B-G depict example embodiments that are intended for (but not limited to) display on a portable computing device such as a smartphone or reader 120, and is referred to herein as the mobile access platform since, in many embodiments (but not all), this is the version customized for everyday use by the patient on his or her mobile device. Information or settings entered into the EIS via the dual access platform (or information processed or determined by the dual access platform) can be downloaded to the mobile access platform through the network as needed, and vice-versa.

FIG. 26 is a diagram depicting an example embodiment of GUI screen for the EIS.

Here, home screen 1600 includes navigation bar 1610, a tool menu (or bar) 2620 with various selectable fields, and also a visit guide selectable field 2600, e.g., in the form of an expandable tab, located to the side of navigation bar 1610.

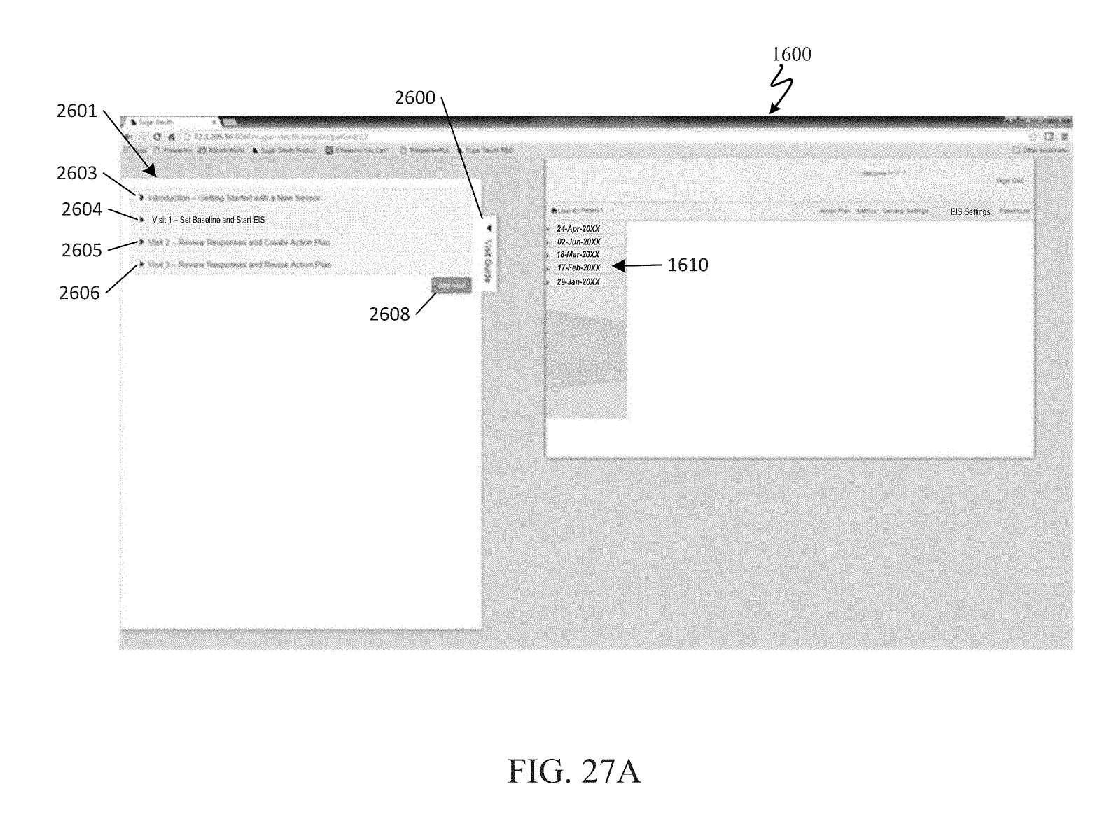

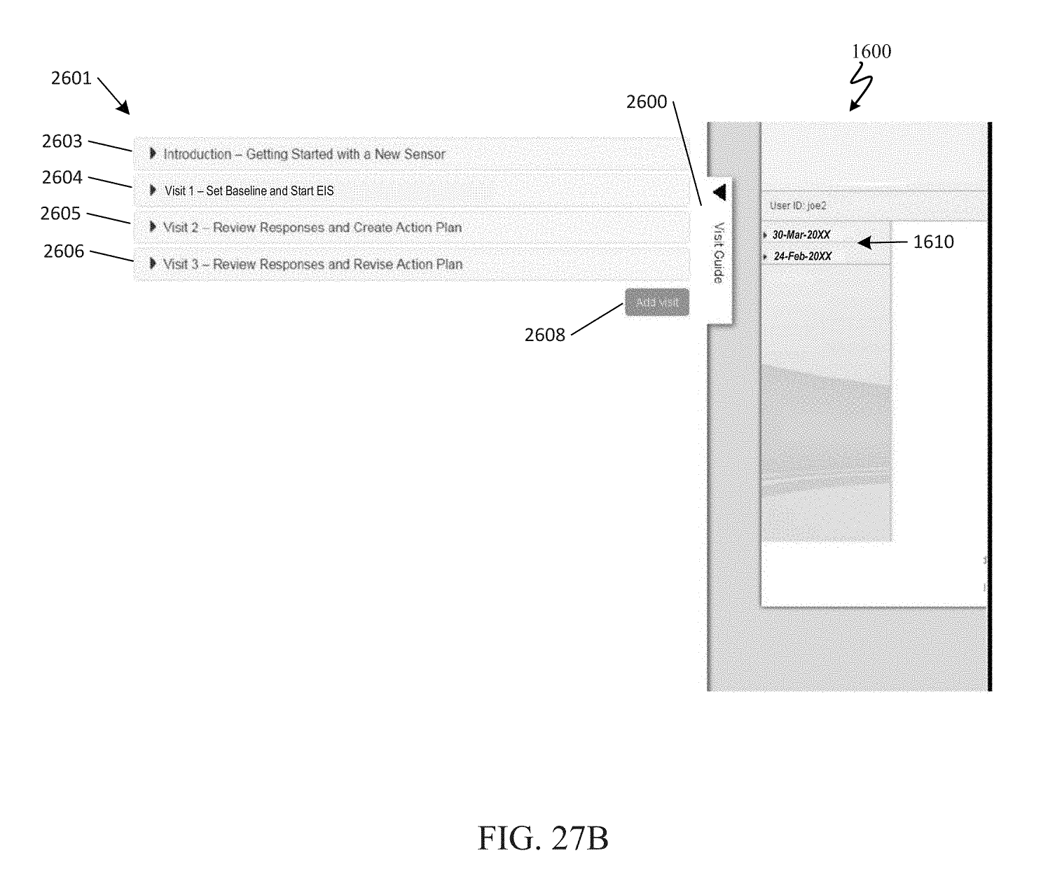

Upon selection of visit guide field 2600, a visit guide menu 2601 can expand across home screen 1600 as depicted in FIG. 27A. In other embodiments, the EIS can transition to a separate page for display of the visit guide menu 2601. Visit guide menu 2601 (or simply the “visit guide”) provides a roadmap for the HCP to follow that facilitates the more involved process. Visit guide menu 2601 provides instruction for training HCP's to follow the full EIS process, but it also does not restrict the experienced HCP's from skipping steps or otherwise changing the process they follow.

Visit guide menu 2601 is shown in greater detail in FIG. 27B. Menu 2601 can include multiple expandable fields 2603-2606 where each expandable field corresponds to a particular visit with the HCP or other stage of the investigation and treatment process. Visit guide menu 2601 can show all the visits on one screen so that the user can see the instructions in the context of all the visits. Each visit corresponds to an expandable field 2603-2606 and the sub-fields within each visit can also be expandable to allow the optimization of space so the user can focus on the current section of the visit instructions while maintaining the overall context. In some embodiments, when one sub-field is expanded the others collapse, but the higher level fields 2603-2606 are still visible to maintain context. This format facilitates guidance but does not restrict the user to a specific sequence of steps, such as is often required by a “wizard” design.

Here, field 2603 corresponds to an introduction of sensor usage for the HCP and/or patient that may occur during a first consultation between the HCP and patient, after which the patient may wear a sensor control device 102 for a period of several days or weeks. Field 2604 corresponds to a first visit for the review of the patient's analyte data (e.g., the first visit after the initial consultation) between the HCP and patient where a first baseline data range is selected and the EIS is used to generate reports from that first baseline data set. Field 2605 corresponds to a second visit between the HCP and patient where the patient's responses to identified episodes are reviewed and an action plan is created. Field 2606 corresponds to a third visit between the HCP and patient where again, the patient's responses to identified episodes are again reviewed and the action plan is revised if necessary. Selectable field 2608 allows the user (e.g., HCP or patient, etc.) to add one or more additional visit fields to menu 2601.

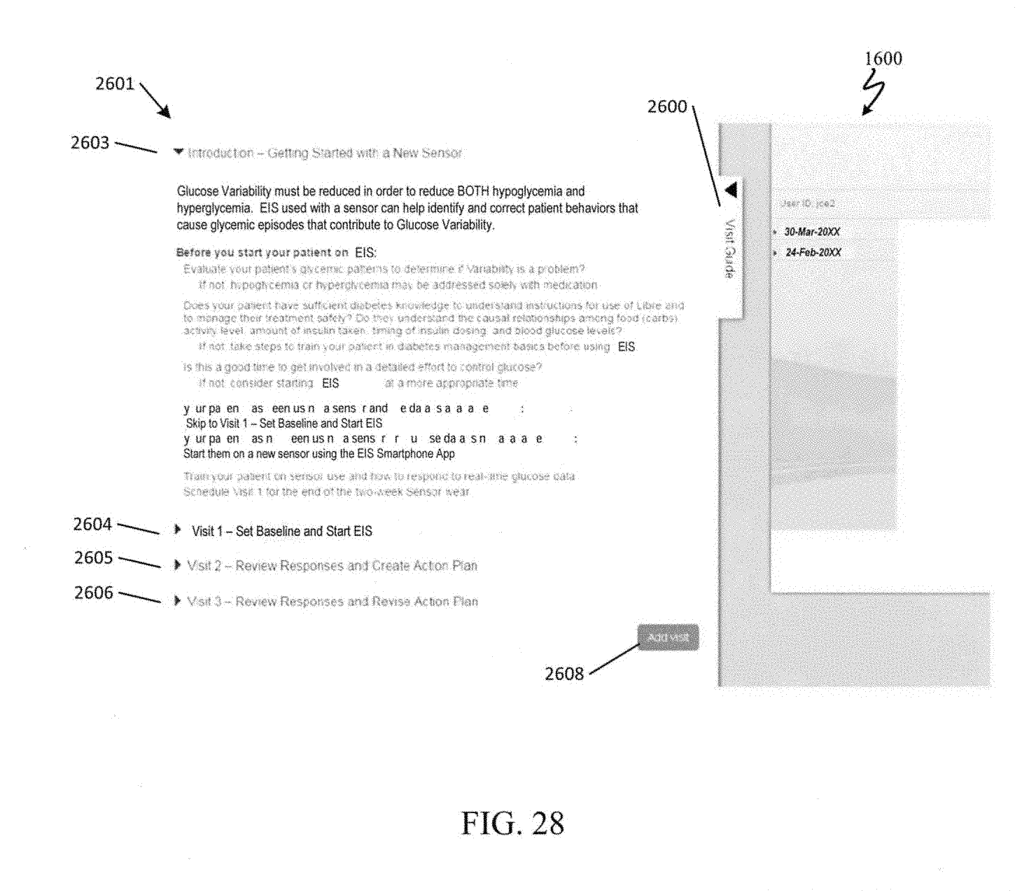

Selection of expandable field 2603 displays a detailed guide that instructs the HCP how to precede through an initial consultation with the patient, as depicted in FIG. 28. This detailed guide can address a number of considerations such as inquiring whether the patient's diagnosis reveals that glycemic variability is a problem, e.g., is above accepted thresholds for variability, inquiring whether the patient has sufficient knowledge of operation of system 100 to manage treatment safely, inquiring whether the patient understands causal relationships between carbohydrate intake, activity level, insulin dose and timing of administration, and blood glucose levels, and inquiring whether this is an acceptable time for the patient to begin a detailed effort to control glucose. If the patient has been using system 100 to collect data already, then guide 2603 can instruct the HCP to proceed to the Visit One guide 2604. If the HCP determines that the patient is ready to begin a detailed effort to control glucose, then the HCP can begin the patient on a data collection routine using system 100 and the EIS on the patient's reader device 120.

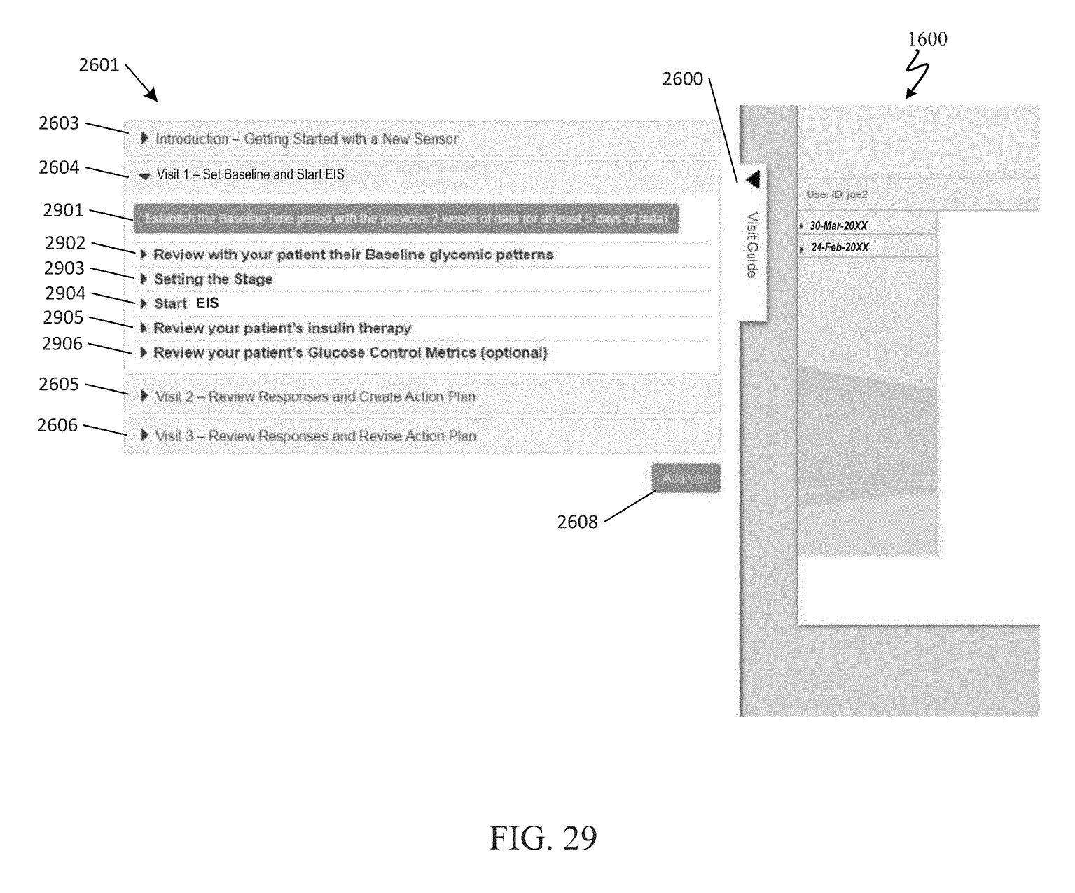

When ready to precede to Visit One field 2604, selection of that field displays an expanded menu including selectable fields 2901-2906, as depicted in FIG. 29. Selectable field 2901 allows the user to select the first period of baseline data for analysis using the EIS. The baseline data can be selected using a data selection interface such as that described with respect to FIGS. 17A-17B. A period of one or more days, one or more weeks, or longer can be selected and designated as the baseline period.

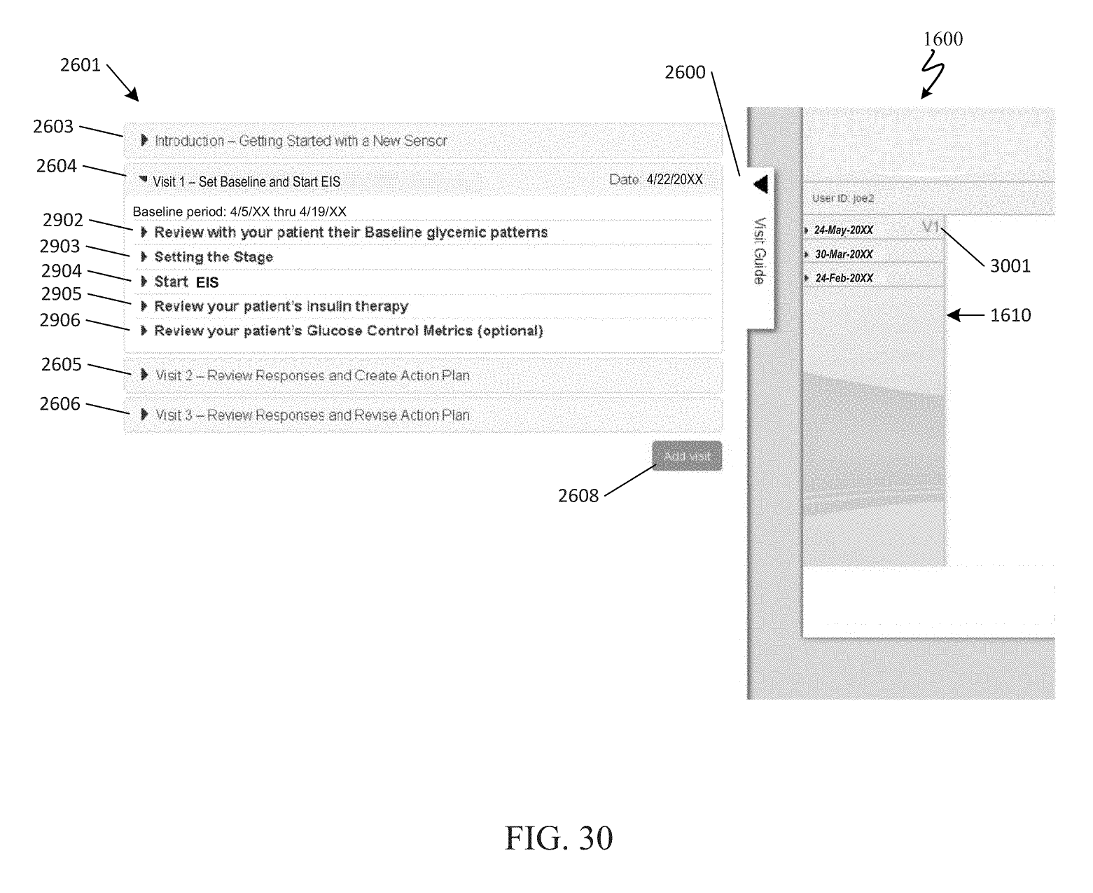

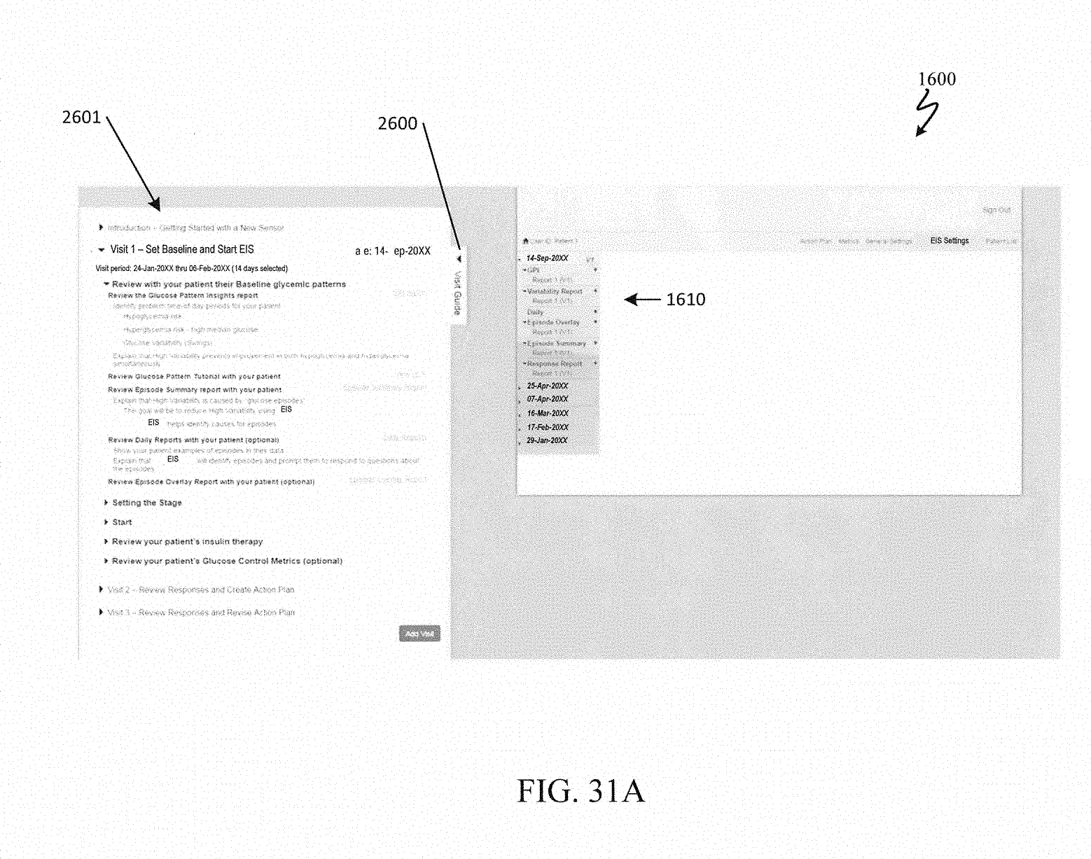

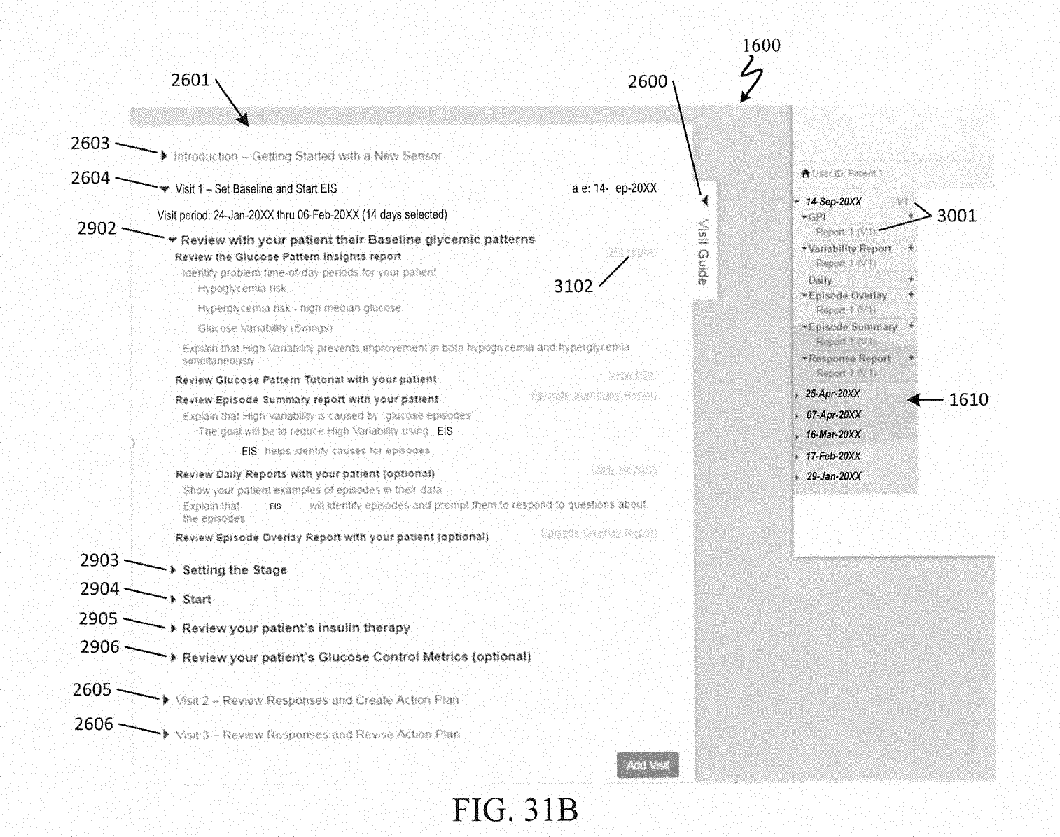

Once the first baseline data range is selected the selectable field 2901 is replaced with an indication of the date range selected as depicted in FIG. 30. A new session is indicated in navigation bar 1610 corresponding to the data established as the baseline. An indicator 3001 is displayed to indicate that the session was created during Visit One. This indicator 3001, when displayed for multiple sessions across multiple visits, allows the users to readily identify which sessions correspond to which visits. Also, once the date range for baseline data is selected, all reports which require a date range selection are created and made available for viewing with associated indicators 3001 (see FIG. 31B) and additional links that provide access to specific reports are enabled in the visit script (e.g., within expandable fields 2902-2906).

The selectable fields 2901-2906 are arranged in sequence according to the order in which each step should occur. For example, the first step was to establish a baseline data using field 2901. In some embodiments, the subsequent fields 2902-2906 can be locked out (prevented from expanding) until the baseline data range is established. Upon completion of that step 2901, the HCP can select the subsequent field 2902 that corresponds to the step of reviewing the patient's baseline glycemic patterns. As depicted in FIGS. 31A-B, selection of field 2902 expands a menu with a list of suggested actions that the HCP can take including, reviewing various reports generated by the EIS with the patient and reviewing informative or tutorial documents. Each action has a corresponding link 3102 that can be selected to retrieve the recommended report or other document.

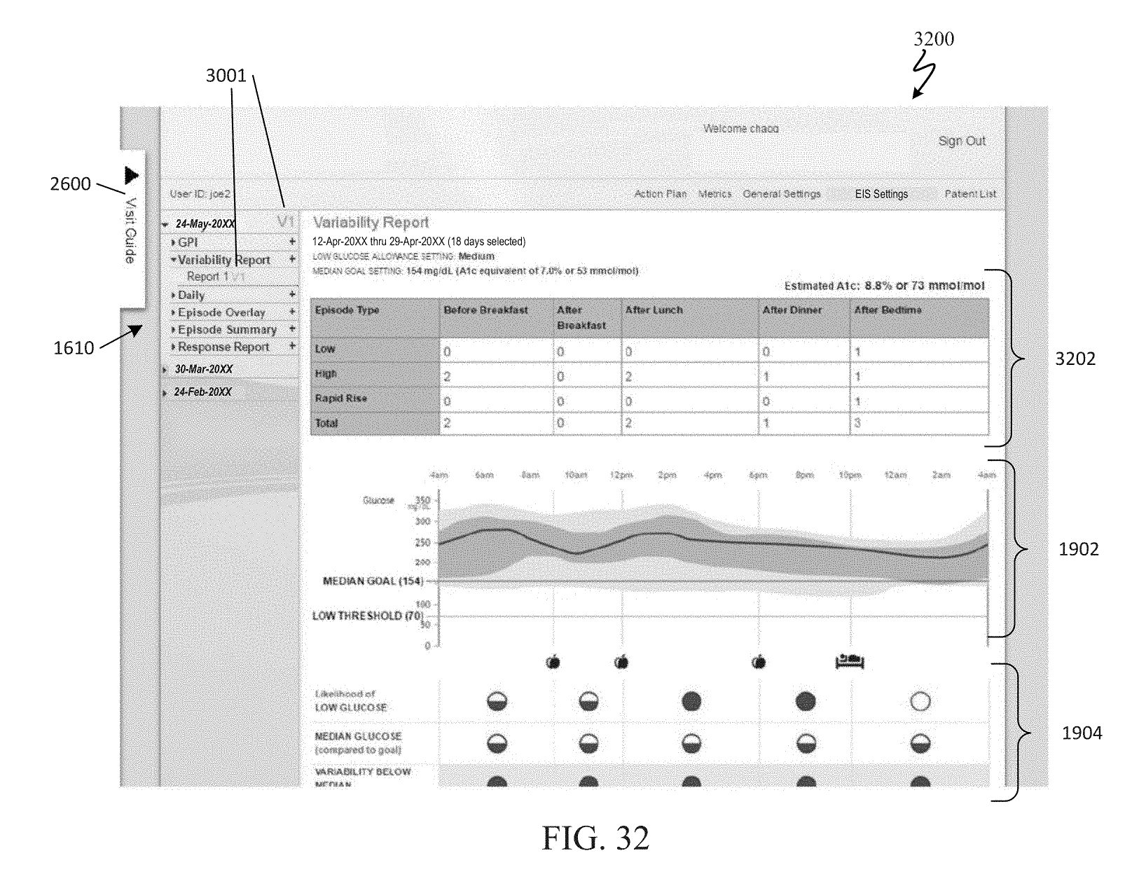

For example, selection of the link 3102 for a variability report will cause the display of screen 3200 showing the variability report created from the baseline data as shown in FIG. 32. In the navigation bar 1610 each of the available report types are displayed as selectable fields. Underneath the variability field is another selectable field corresponding to the identification of this report as “Report 1” with a corresponding indicator 3001 that indicates this report was generated during Visit One. The variability report itself includes first region 1902 (see also FIG. 19A) that displays the ambulatory glucose profile (AGP) of the user and second region 1904 (see also FIG. 19A) that displays assessments of the severity of certain conditions related to the user's diabetes. Above region 1902 is a region 3202 with a table of episode types and the number of instances that each episode type occurred in one of a number of defined time periods, such as before breakfast, after breakfast, after lunch, after dinner, and after bedtime.

The display of all other reports can also occur with the inclusion of indicators 3001 in navigation bar 1610. For example, generation of daily reports for each day in the baseline period (see FIG. 18A) will cause indicators 3001 to be displayed in the navigation bar for each daily report created or viewed during Visit One.

The next step in the Visit One itinerary corresponds to selectable field 2903, which displays to the HCP a series of questions or factors to be considered to assess the patient's level of engagement with the investigation process and the patient's motivation and interest in identifying the causes for glycemic episodes. Field 2903 also displays questions to assist the HCP in identifying whether outside factors in the patient's life are too significant to warrant engaging in a detailed behavioral analysis and modification routine. Overall, this narrative assists the HCP in a conversation with the patient during Visit One and helps ensure that the HCP does not embark in an effort intensive regimen to collect additional data about the patient's behavior and seek to modify it without ample commitment by the patient.

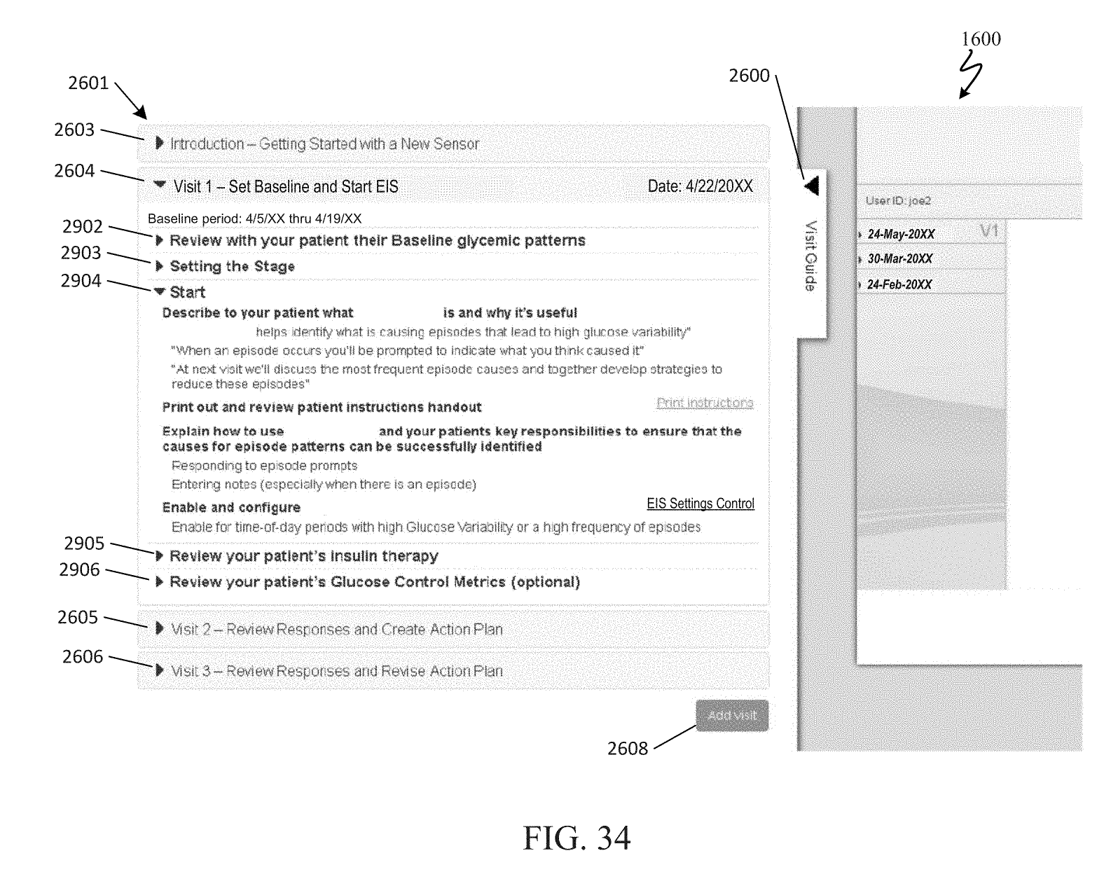

The next step in the Visit One itinerary corresponds to selectable field 2904, selection of which displays a drop down menu with a list of steps to precede through sequentially that efficiently begins the process of using the EIS. For example, as shown in FIG. 34, the first step can be to describe to the patient what the EIS is and why it is useful, and a sample script is provided to accomplish the same. The next step can be to print and review a document containing patient instructions for use of the EIS and a selectable link is provided for the same (“Print instructions”). Next, the drop down menu 2904 instructs the HCP to explain how to use the EIS and the patient's key responsibilities to ensure that the causes for episode patterns can be successfully identified.

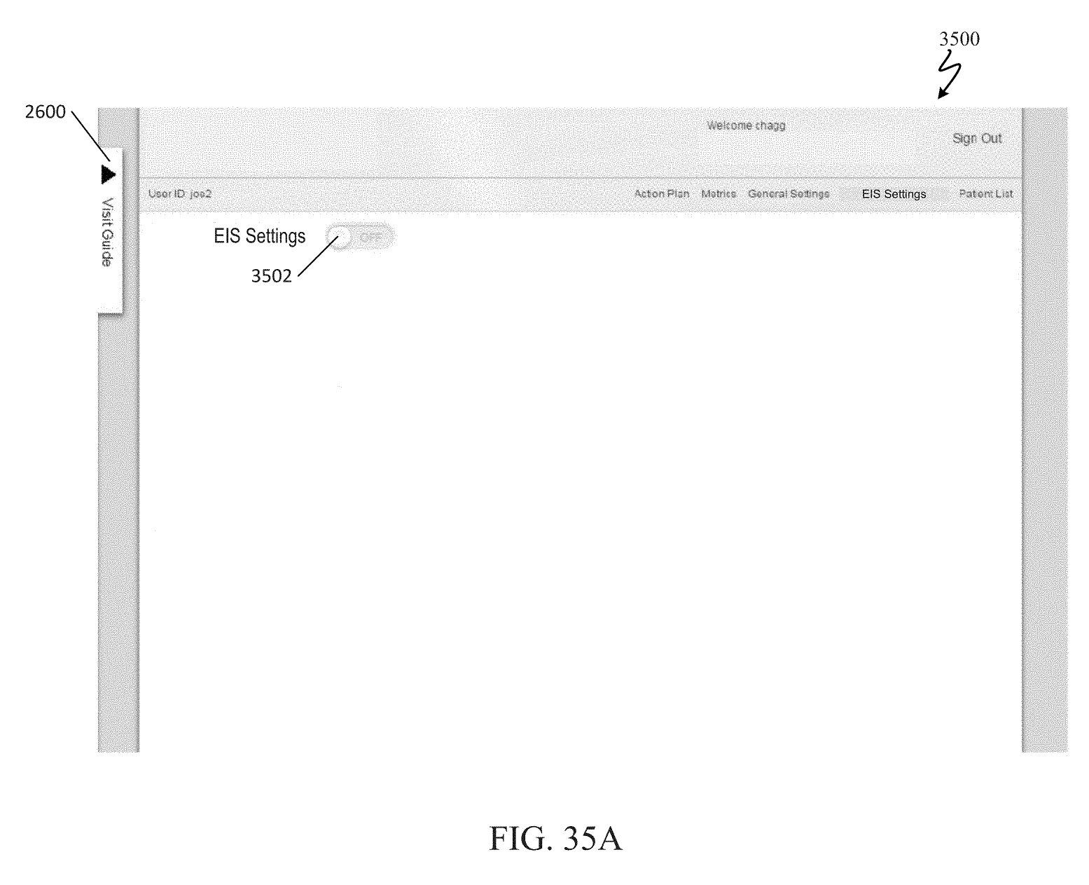

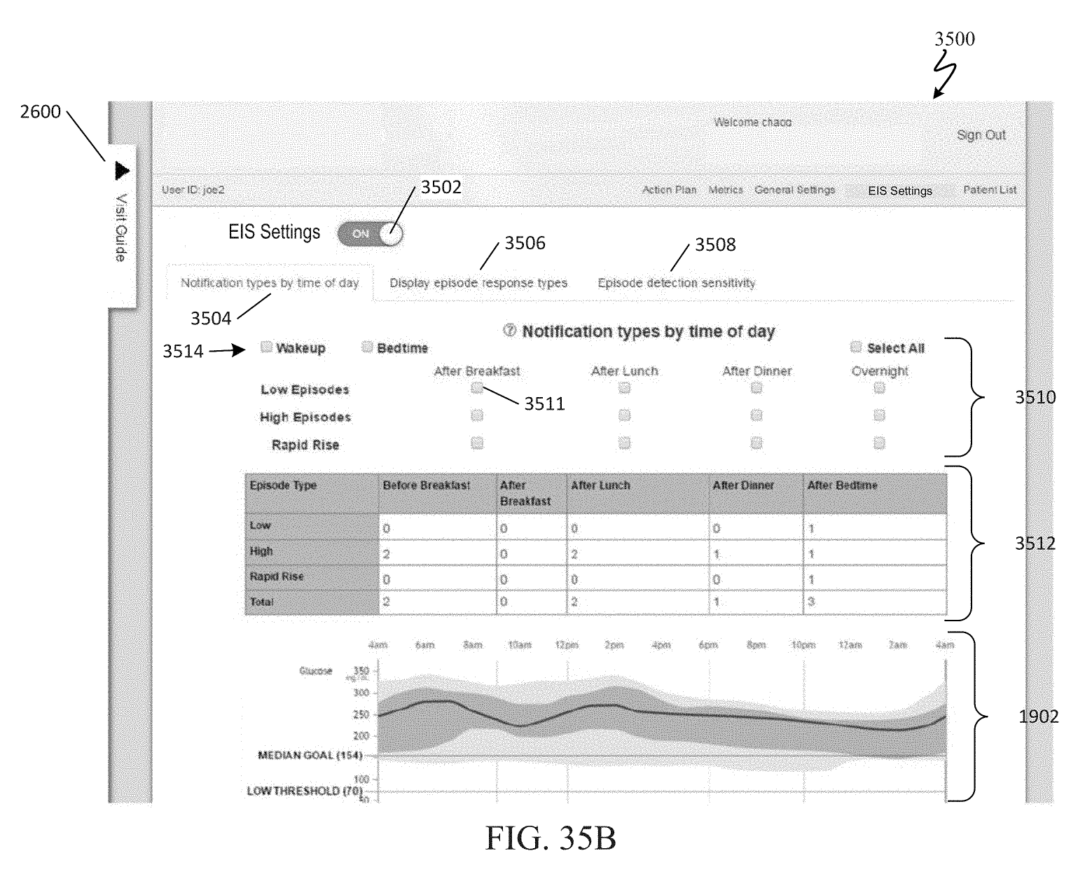

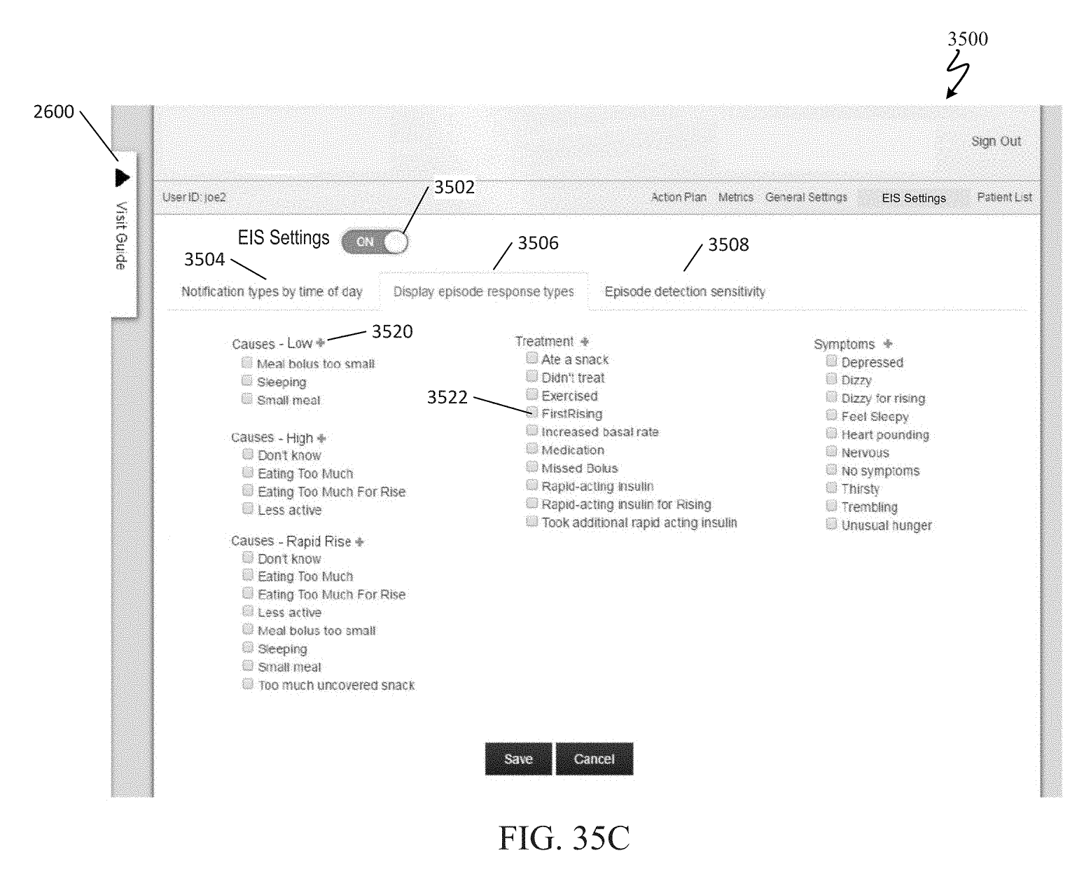

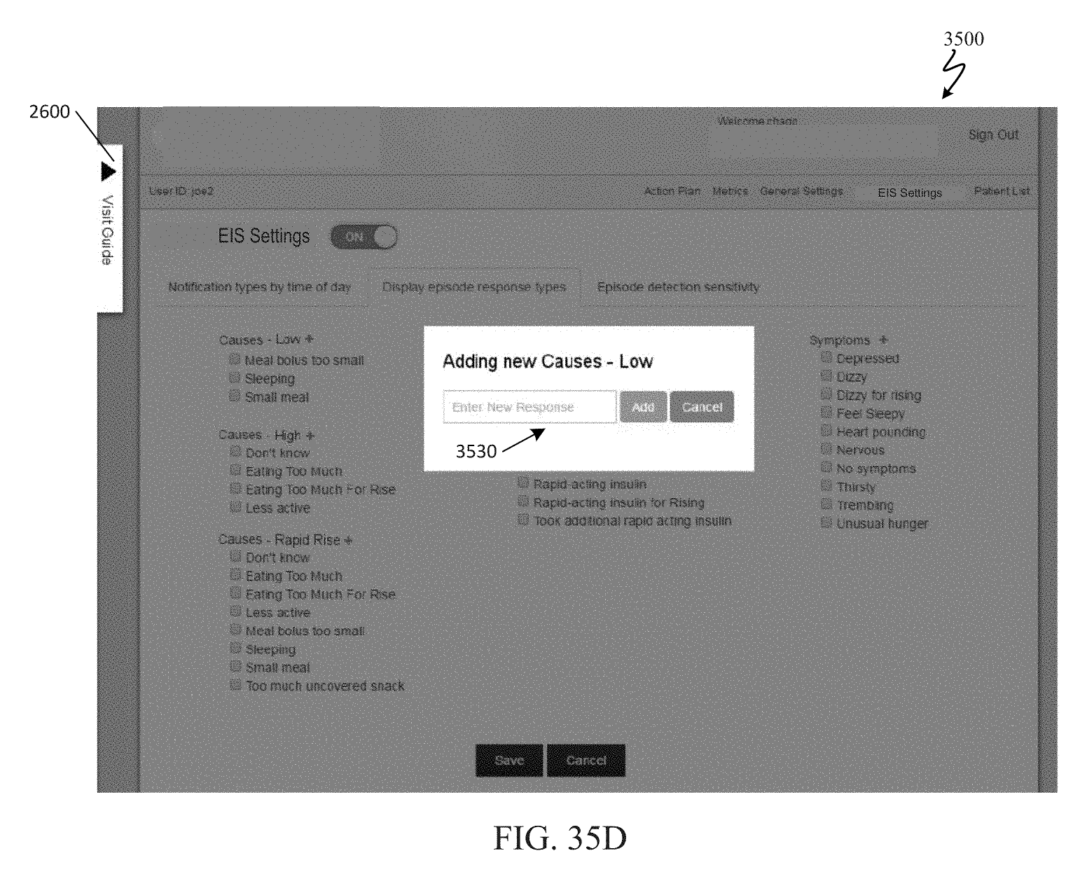

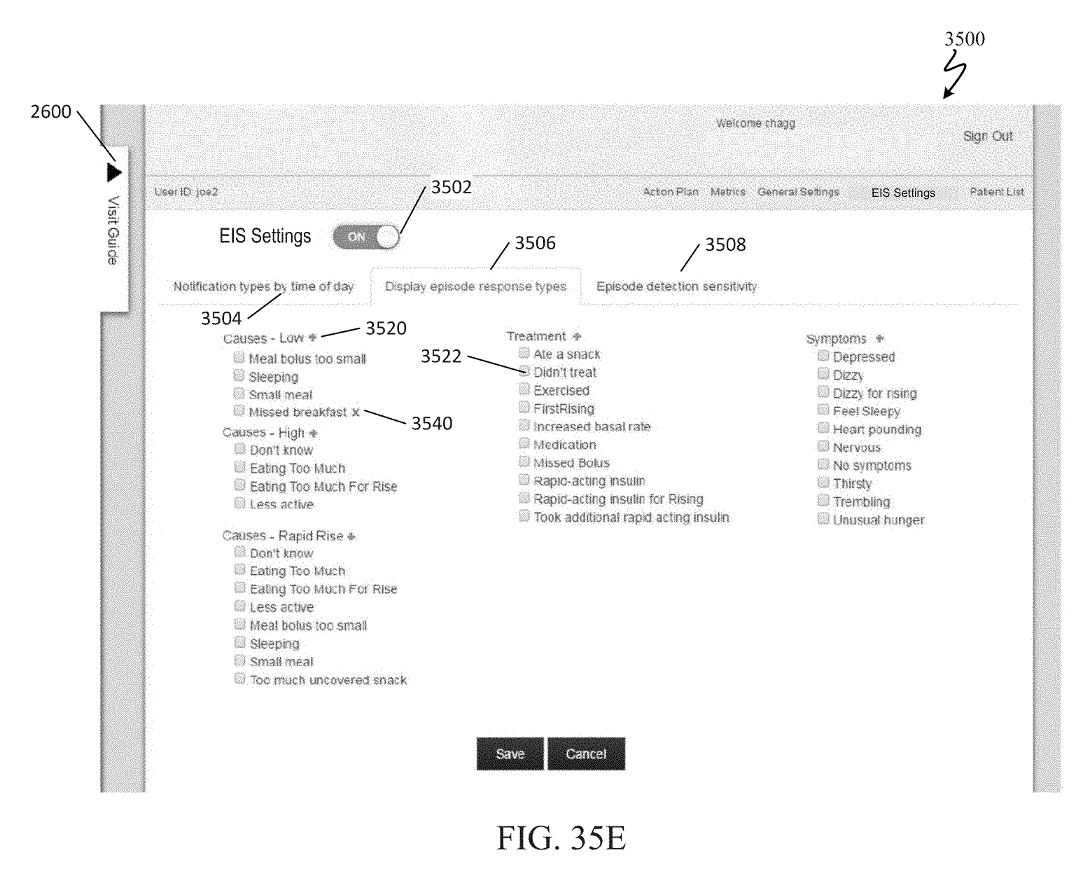

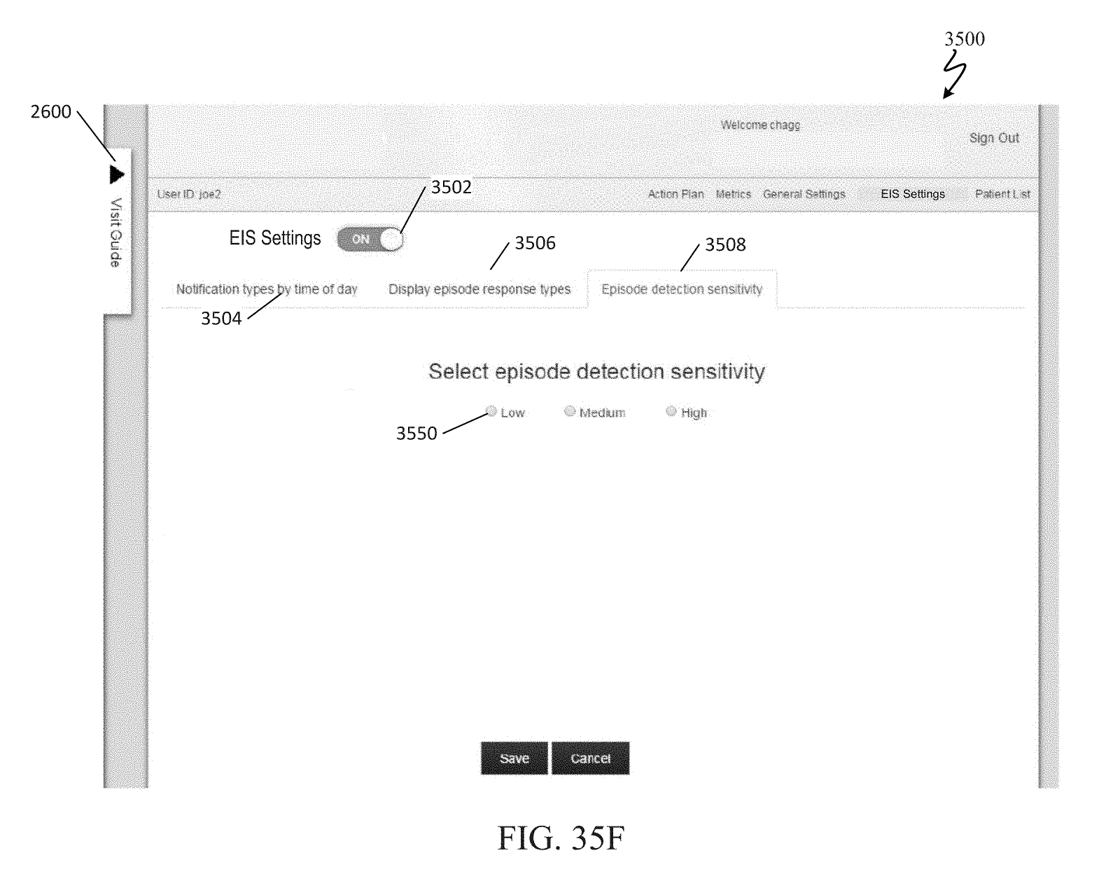

Finally, menu 2904 instructs the HCP to enable and configure the settings for the EIS and a link to the EIS settings control panel is provided (“EIS Settings Control”). Selection of this link causes the display of a settings control screen 3500 as depicted in FIG. 35A. In this embodiment, a selectable field 3502 is present in the form of a toggle switch that determines whether the adjustable portion of the settings panel 3500 is shown. Field 3502 defaults to the off position shown here and selection of (e.g., clicking) field 3502 causes the settings panel to be displayed as shown in FIG. 35B.

Settings panel 3500 can include multiple interfaces, and, in this embodiment, there are three selectable interfaces in the form of tabs 3504, 3506, and 3508. Selectable interface 3504 is selected in FIG. 35B and controls which notification types will be issued to the patient during which times of day. Interface 3504 includes a first region 3510 that contains selectable fields 3511 in the form of check boxes, each corresponding to a particular episode type and time-of-day.

For example, three episode types are shown: low glucose episodes (“Low Episodes”), high glucose episodes (“High Episodes”), and rapid rise in glucose level episodes (“Rapid Rise”). Each of these episode types has a selectable field 3511 in each of the various times of day, which, as shown here, can be displayed in groups according to the time of day relative to a meal or the user's sleep schedule (e.g., after breakfast, after lunch, after dinner, overnight, etc.). Selection of one of these fields 3511 can enable or disable the generation of notifications for that episode type and time-of-day. When one or more selections are made on the dual access platform, those selections are downloaded to the mobile access platform on the smartphone reader 120 responsible for generating the respective notifications for the associated episode in the proper time-of-day period. For example, when the Save field at the bottom of the screen for any settings tab is selected (bottom of screen not shown in FIG. 35B), these settings are sent to the patient's smartphone app, which will commence to provide notifications as configured. In other embodiments, different groupings based on time can be used, for example, time periods corresponding to each hour of the day, two hour periods of the day, and so forth.

Selectable fields 3514 are, in this embodiment, labeled as “Wakeup” and “Bedtime.” Additional notifications can be enabled using these selectable fields 3514, where these additional notifications are initiated by time-of-day rather than glucose episode type. For example, bedtime notifications can occur on smartphone 120 at the indicated bedtime setting time and wakeup notifications can occur at the indicated breakfast setting time. In some embodiments, the bedtime notification can include questions for the patient to answer about overnight glucose risk factors and any treatment action that had been performed. In some embodiments, the wakeup notification can include questions about whether the treatment was appropriate. In both examples, the EIS notification can provide access to a mechanism or link by which the patient can enter a response that will then be recorded by the local memory and uploaded to the dual access platform if necessary.

To assist the HCP and making the selections within region 3510, a summary of the episode types that occurred in the baseline data (or other selected group of data) is displayed in a region 3512 in the form of a table. The episode occurrences are aligned by episode type and by time-of-day in the same fashion as within region 3510. Thus, identification of time periods and episode types that may result in the most episode occurrences can be readily identified and used to complete the notifications selection fields 3511 in region 3510. The table 3512 can provide the HCP guidance about which time-of-day periods are most important to enable notifications, for instance, periods with the most episode occurrences. This can be important in certain embodiments for reducing the burden on the patient such that the patient can focus on answering prompts only when necessary.

Beneath region 3512 is an AGP display 1902 also with the data aligned by time period in the same fashion as regions of 3510 and 3512. Although not shown, a region 1904 can also be included with the indicators aligned again in the same fashion according to time. Thus, the settings control panel can simultaneously display the patient's data in tabular and graphical form in a manner aligned with the adjustable settings control region 3510, which can greatly facilitate the process of setting a notification functionality to the patient.

Selection of interface (tab) 3506 causes the display of a setting control panel for the various indications that will be displayed upon detection of a particular episode type as shown in FIG. 35C. For example, when an episode of each episode type (e.g., low, high, rapid rise) occurs, the user will be queried to enter a potential cause of the episode type, a treatment action that was performed and response to the episode occurrence, and symptoms that accompanied the episode occurrence.

Interface 3506 allows the user to set the potential causes, potential treatments, and potential symptoms that will be displayed to the patient on reader device 120 when a particular episode is detected. Selectable fields 3522 are present with one field 3522 associated with each potential cause, treatment, and symptom, such that selection of various fields 3522 will determine which potential causes, treatment, and symptoms are displayed when each episode is detected. In this embodiment, the causes are further subdivided by episode type although this is not a requirement. Similarly, the potential treatments and symptoms can also be subdivided by episode type. In some embodiments, by default, all fields 3522 are selected. When the Save field at the bottom of the screen is selected then these settings can be automatically downloaded to the mobile access platform on the patient's smartphone 120, which will commence to provide notifications as configured.

When interface 3506 is displayed a default list of selectable fields is presented corresponding to common causes, treatments, and symptoms. If the user wishes to add additional fields then an add field 3520 can be selected which will cause the display of an interface 3530 that allows the user to enter a new cause, treatment, or symptom for display in settings control panel 3506. For example, selection of the add field 3520 for “Causes—Low” will cause interface 3530 to be displayed as depicted in FIG. 35D. Entry of a new selectable response in to interface 3530 will cause that new response to the added to the list of selectable fields in interface 3506 as depicted in FIG. 35E. Here, the new selectable response “Missed Breakfast” that was entered via interface 3530 is now shown along with a selectable field 3540 that allows deletion of that entry if desired. The user can then select each and every potential response that should be displayed upon detection of an episode by selecting (e.g., checking) the corresponding field 3522.

Selection of interface 3508 causes display of a screen with selectable fields 3550 that allow the user to adjust the episode detection sensitivity for the EIS as depicted in FIG. 35F. Here, selection of a low sensitivity will cause the EIS to utilize episode detection thresholds of a relatively high magnitude resulting in the detection of relatively fewer episodes. Conversely, selection of a high sensitivity will cause the EIS to utilize episode detection thresholds of a relatively mild magnitude resulting in the detection of relatively more episodes. Selection of the medium or moderate setting will cause the EIS to utilize thresholds that are between the low and high settings. When the Save button at the bottom of the screen is selected these settings can be sent to the mobile access platform on the patient's smartphone 120, which will commence to detect episodes with the newly configured settings. In this embodiment the interface 3508 allows the user to choose from three different discrete settings, however in other embodiments the episode detection sensitivity can be adjusted with a higher level of granularity, for example by allowing the user to enter numerical values for the various thresholds. In some embodiments, access to settings panel 3500 is only available through the web-based software accessed under the account of the HCP.

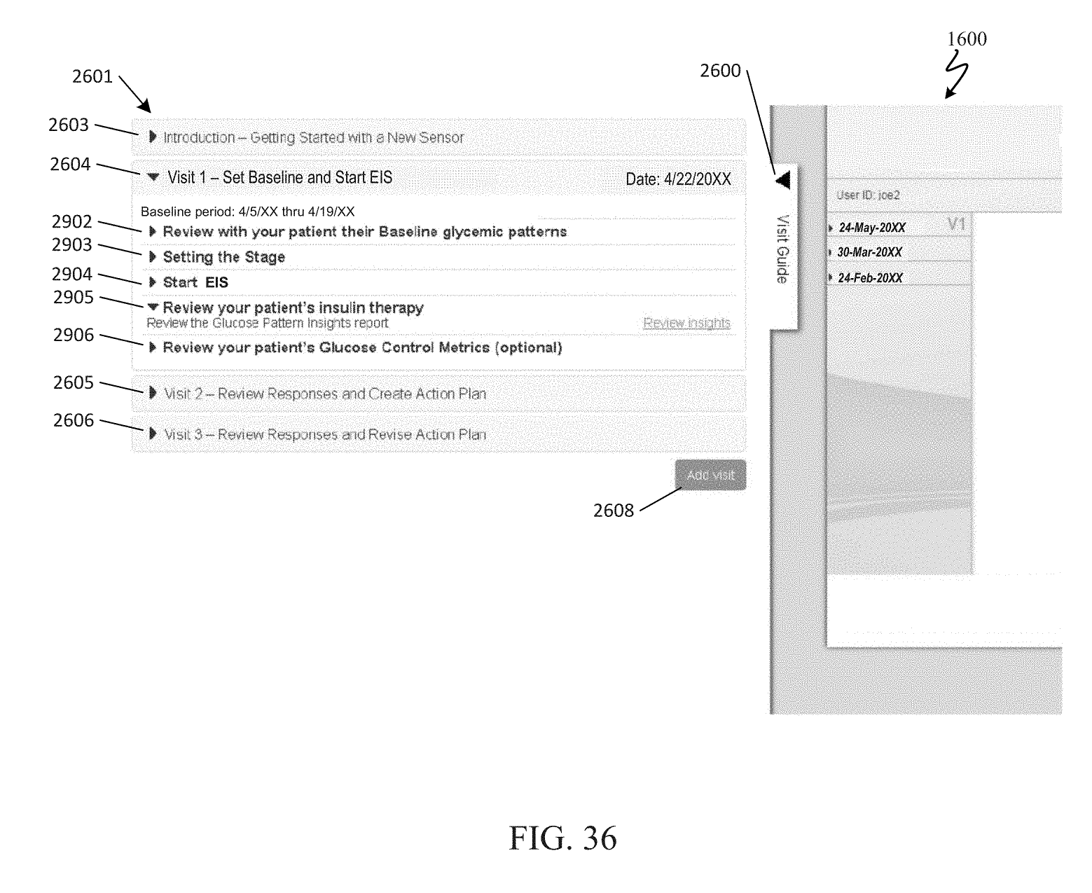

Once the entry of the settings is complete, the user can return to the visit guide by selection of field 2600, which will cause display of FIG. 36 to be shown. Field 2905 corresponds to the next step under Visit One, where it is recommended that the HCP review the patient's insulin therapy with the patient. This can be accomplished by reference to a GPI report (see FIGS. 19A-B) and a link to the same as provided (“Review insights”).

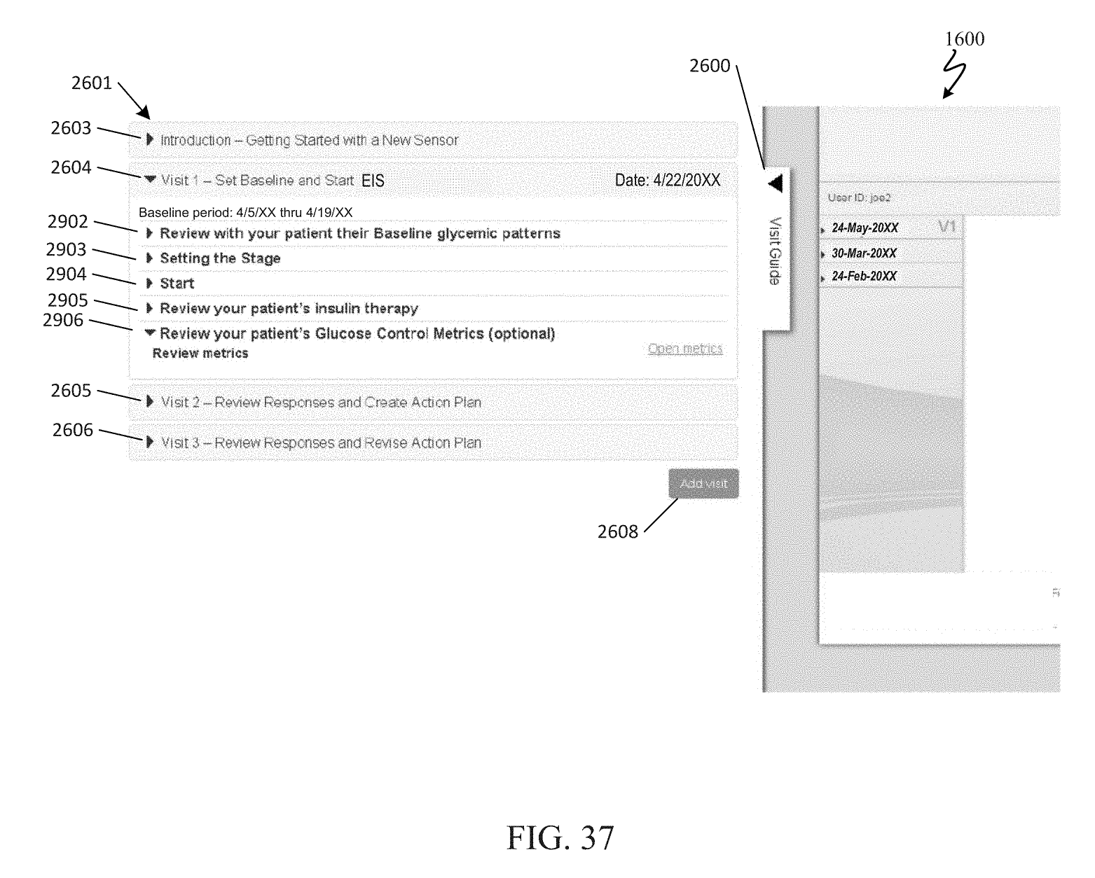

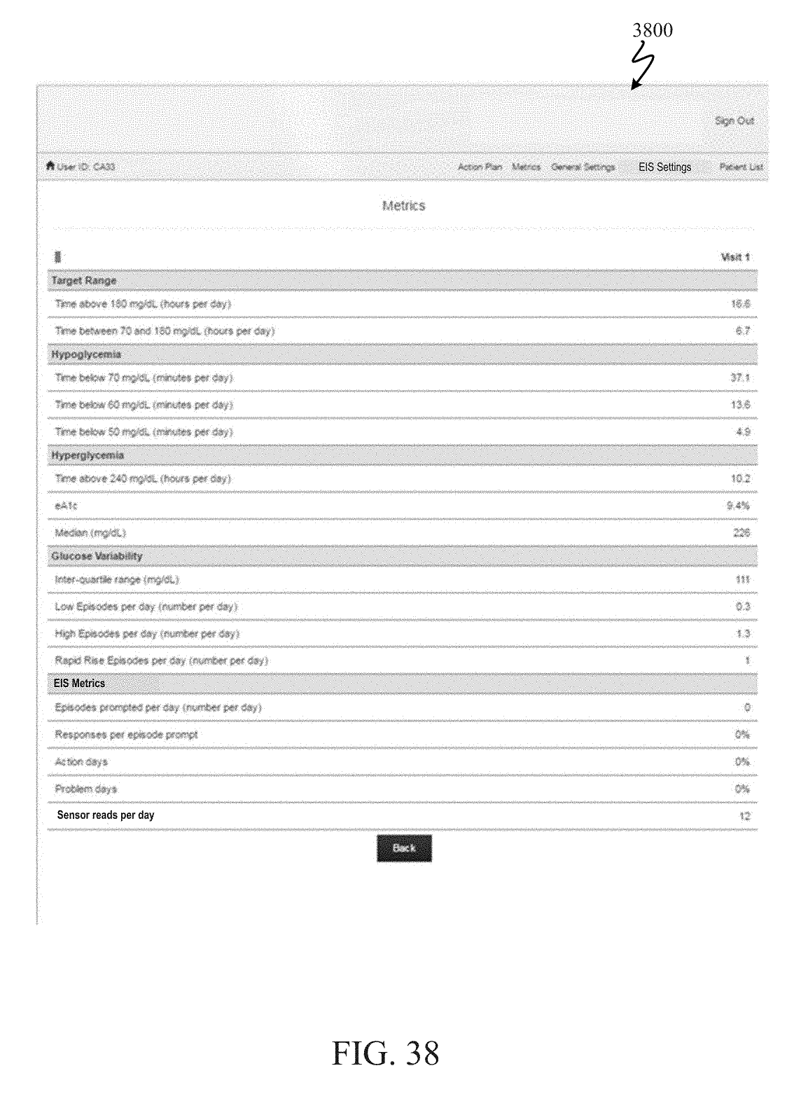

In this embodiment, field 2906 corresponds to the final step of Visit One, where it is recommended that the HCP optionally review the patient's glucose control metrics and includes a link to those metrics (“Open metrics”) as depicted in FIG. 37. Selection of the link causes the metrics report 3800 to be shown as depicted in FIG. 38. Report 3800 can include a list or table of various metrics useful in summarizing the glycemic behavior for the patient in the data of the baseline period reviewed during Visit One.

The various metrics can be grouped according to categories such as target range, hypoglycemia, hyperglycemia, glucose variability, EIS specific metrics, and others. Metrics report 3800 can include numerical results for each metric organized by visit so as to provide a easily digestible summary for the patient and the HCP as to the degree of progress that has occurred in reaching the patient's glycemic targets.

In some embodiments, there are three types of attributes that the EIS associates with each detected episode: a) not prompted, b) prompted and responded to, and c) prompted but not responded to. The first (a) can occur if an episode is detected but not displayed because a more recent episode is detected during analysis of the glycemic data (such as in embodiments where reader device 120 only requests a response for the most recent episode detected during data analysis, but not for prior episodes detected during the data analysis). In some embodiments, metrics report 3800 will aggregate, for each visit baseline data range, the average number of episodes per day that were prompted (attributes b+c), and the fraction of these prompted episodes that were responded to (attributes b/(b+c)). The former metric is useful in tracking if the number of episodes is reducing over time. The latter metric is useful in determining the patient's level of compliance with responding to prompts.

The “Action days” metric counts the number of days in which an action was recorded by the EIS monitoring function (see the goal monitoring prompt 5162 described with respect to FIGS. 51F-G), and displays the result with respect to the number of days in the visit date baseline data range. The “Problem days” metric counts the number of days where the patient, using the EIS app, indicated that the problem occurred at least once, as a response to a detected episode, and displays the result with respect to the number of days in the visit date baseline data range.

The “Sensor reads per day” metric refers to an example embodiment where the sensor control device 102 can be read or scanned by a reader device 120 according to an NFC communication protocol, and upon scanning the sensor control device 102 transmits the collected glucose or other analyte data to reader device 120. Thus this metric tracks how actively the patient scans sensor control device 102 for new glucose data, where a higher frequency of scanning is desirable if prompts are only generated for the most recent episode detected in the scanned data recently obtained by reader device 120. As already stated herein, these embodiments can also be implemented in to analyte monitoring systems 100 where scanning is not required. In those cases, instead of the number of scans, this metric can represent instances where the system detected the data was viewed by the patient.

After the conclusion of Visit One, the patients will continue to use system 100 to collect glycemic data and will use the EIS to generate notifications each time an episode is detected and enter one or more responses about the potential causes, symptoms, and treatments pertaining to a particular detected episode. This process will continue until the patient's next scheduled visit with the HCP, which is referred to herein as Visit Two.

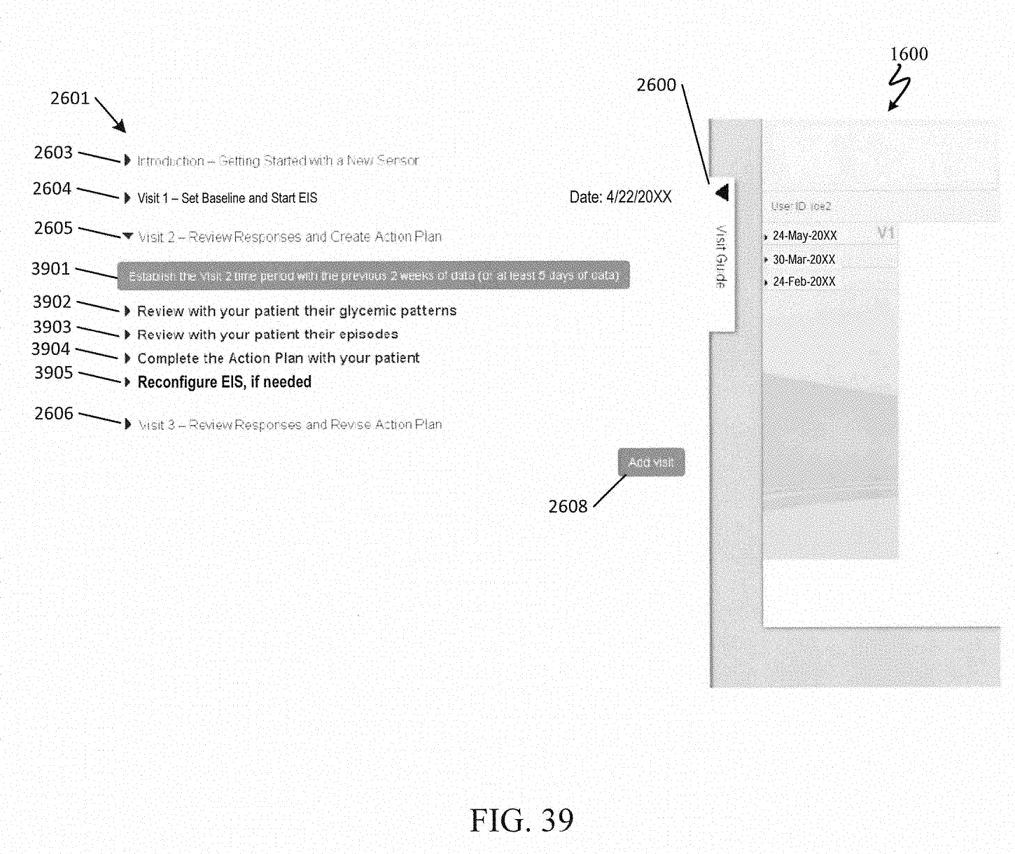

FIG. 39 depicts an example embodiment of the EIS home screen 1600 with visit guide menu 2601 displayed. The expandable menu 2605 corresponding to Visit Two has been expanded and a sequence of selectable fields 3901-3905 are shown, in a manner similar to expansion of the selectable field 2604 for Visit One.

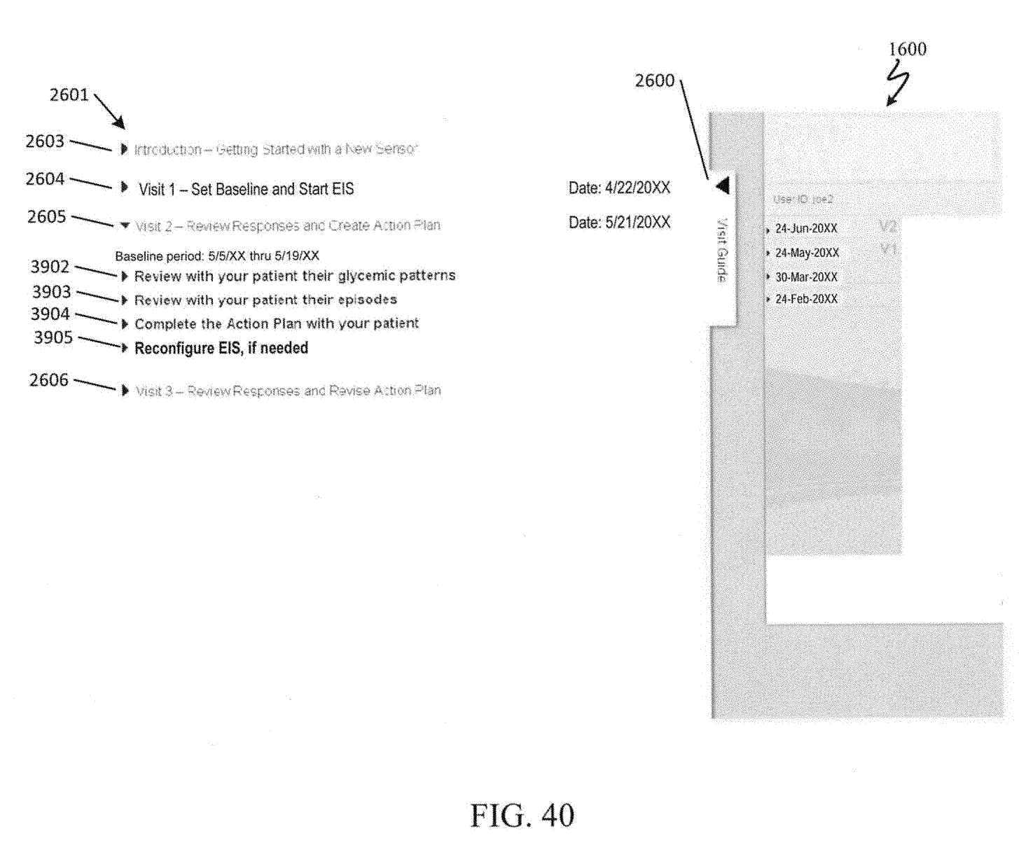

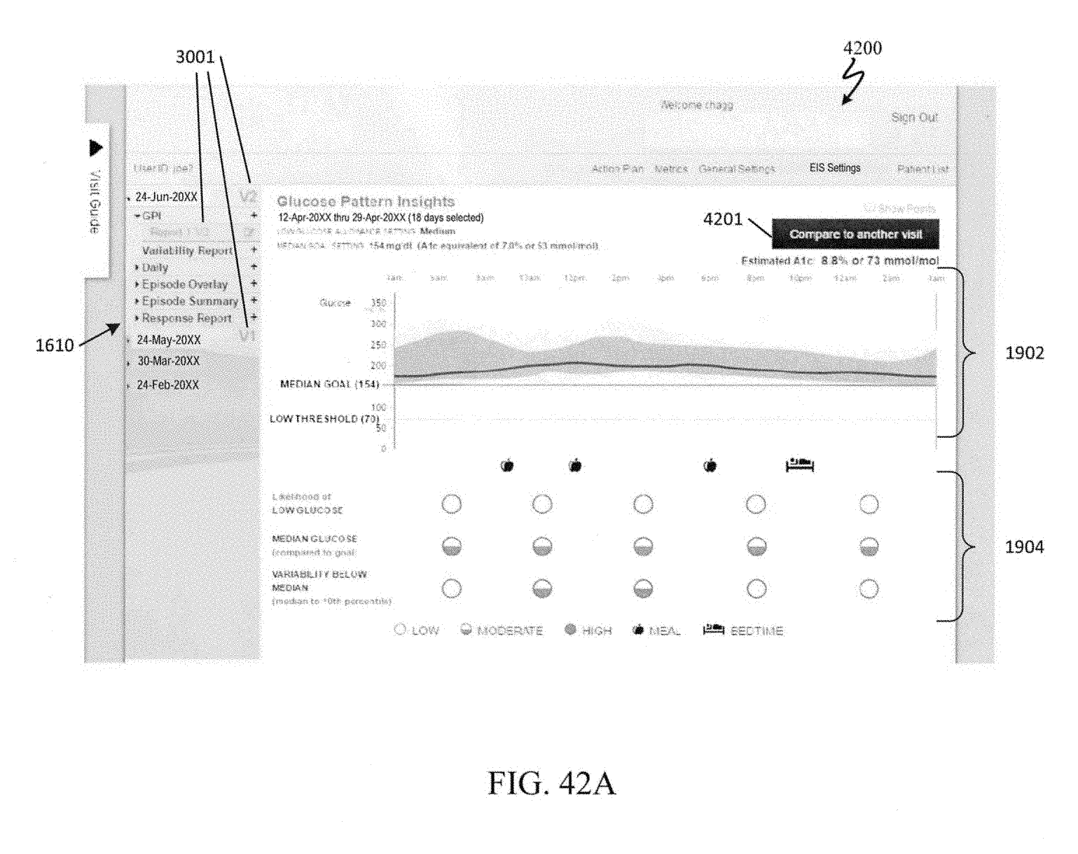

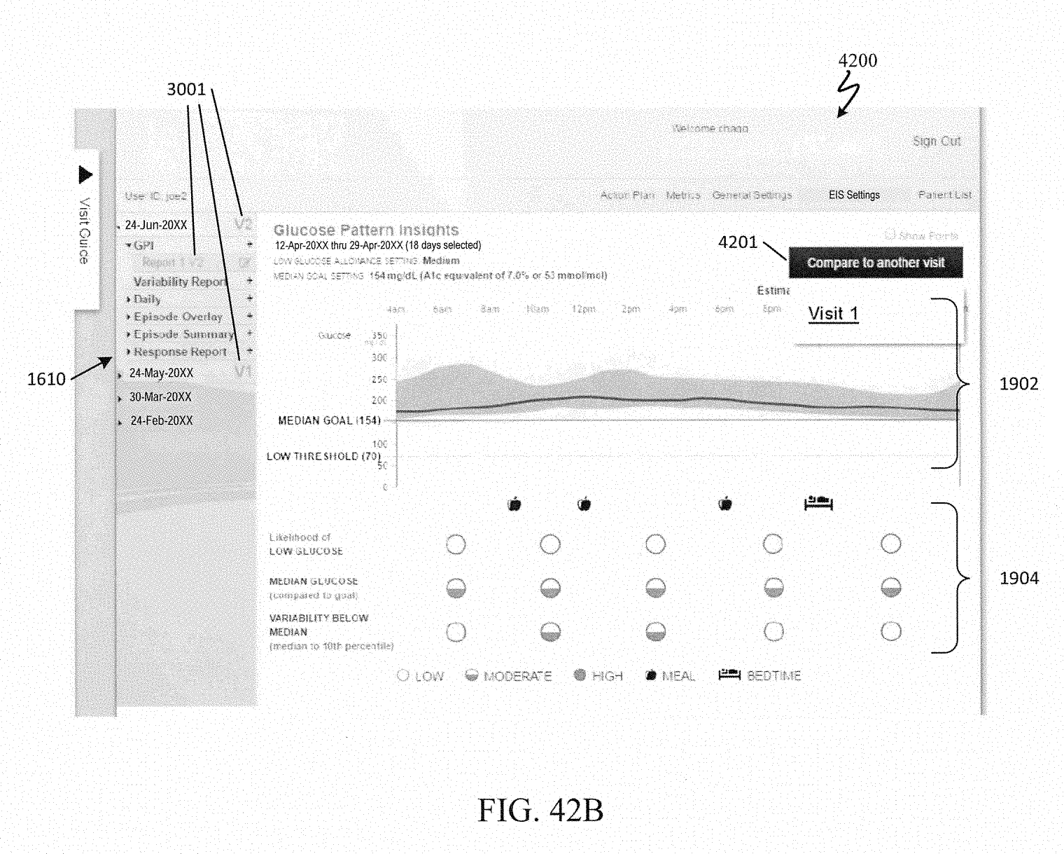

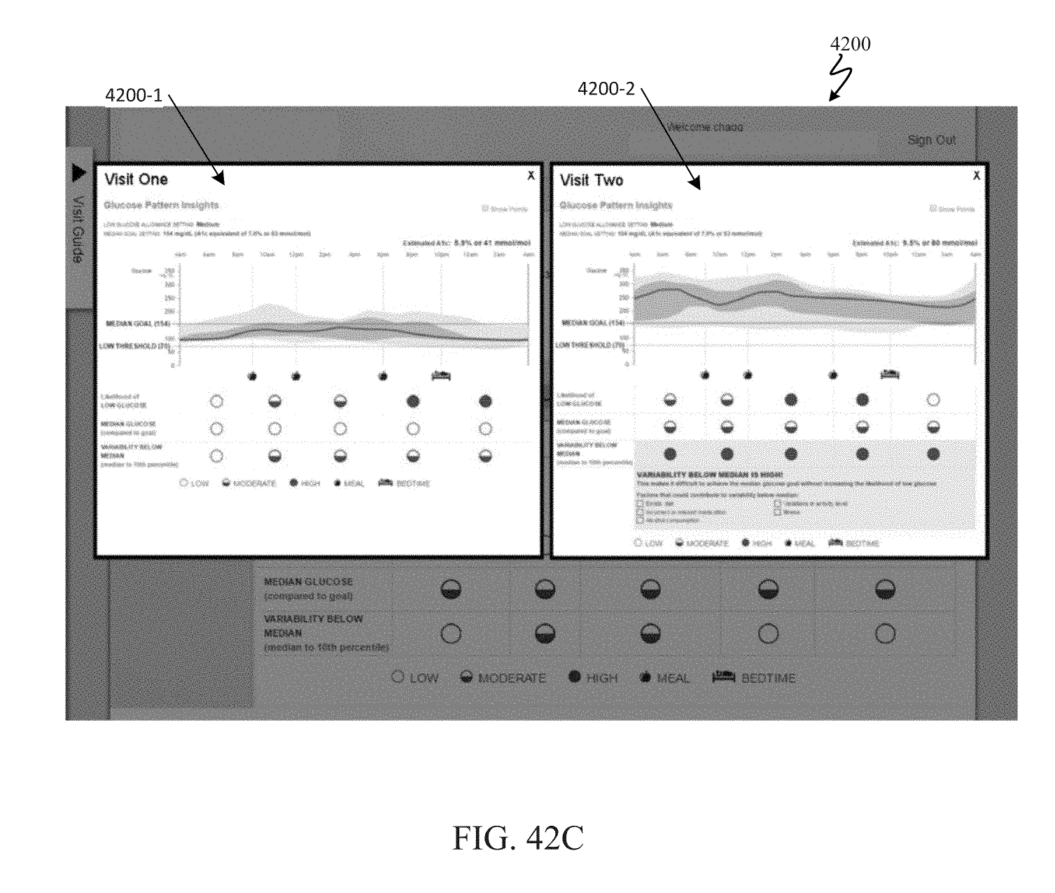

Selectable field 3901 queries the user to establish a data set from the time interval after Visit One that will be used for investigation, analysis, and report generation during Visit Two. Again, this data can be selected using the interface described herein with respect to FIGS. 17A-B. FIG. 40 depicts menu 2601 after the baseline data period is established, where selectable field 3901 has been replaced with an identification of the baseline data time period. Although not shown, in the embodiments described herein a selectable field can also be displayed allowing the user to modify or select a new baseline time period. As with the Visit One example, establishing the baseline time period can cause: a) all reports which require a date range selection to be created and made available for viewing, b) additional links that open up specific reports to be enabled in the visit script, c) the session date in the navigation bar to be generated with an indication 3001 of the visit number (see FIG. 42A) and the current date to be shown on the script for that visit.

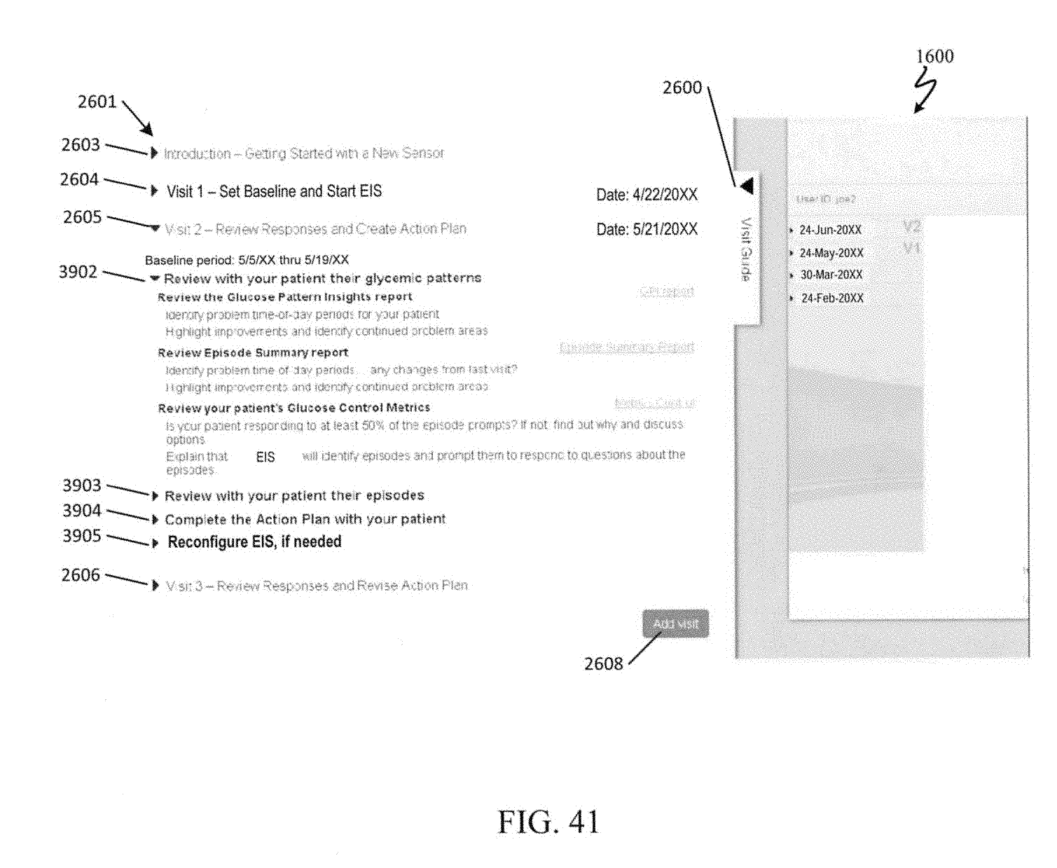

FIG. 41 displays menu 2601 after expansion of selectable field 3902, where the HCP can find a series of suggestions, or a proposed itinerary, for conducting a first part of the Visit Two consultation where the patient's glycemic patterns are reviewed. The first suggested action is to review the GPI report with the patient and a selectable link to the “GPI report” is provided.

Selection of the “GPI Report” link can cause the display of any embodiment of the GPI Report described herein. FIG. 42A depicts one example embodiment of GPI report 4200 that can be displayed. Here, navigation bar 1610 shows each of the various sessions that have been conducted with the patient and the current session is expanded with a listing of the various reports that can be accessed. Within navigation bar 1610 the GPI report has been expanded and the current report is identified along with an indicator 3001 that corresponds to Visit Two (“V2”). A similar indicator 3001 is also shown adjacent to the current session, and an indicator 3001 is shown adjacent to the Visit One session (“V1”).

In this embodiment, GPI report 4200 again includes regions 1902 and 1904 populated with data and graphics generated from the newly selected Visit Two baseline time period. A selectable field 4201 is present that provides the option of comparing this instance of GPI report 4200 with another GPI report previously generated, such as from a prior visit or session. In this embodiment, selection of field 4201 causes the display of a drop down menu with a link to the GPI report from the prior visit (“Visit One”) as depicted in FIG. 42B (although more than one option for comparison will be available in other instances).

Selection of that link causes the EIS to display the GPI report 4200-1 from the first visit alongside the GPI report 4200-2 from the second visit. These reports 4200-1 and 4200-2 can be displayed horizontally (side-by-side as shown here) or vertically (top-to-bottom, not shown) as pop-up windows that overlie screen 4200 as depicted in FIG. 42C. The capability to present the user with a quick and convenient tool for side by side report comparison is yet another feature of the EIS that greatly improves the efficiency of the consultation between the HCP and patient and that greatly improves the ability of the HCP and/or patient to understand the degree of improvement (or lack thereof) in the patient's glycemic profile that has occurred across visits. This selectable field 4201 and report comparison functionality can be included in all embodiments of reports described herein.

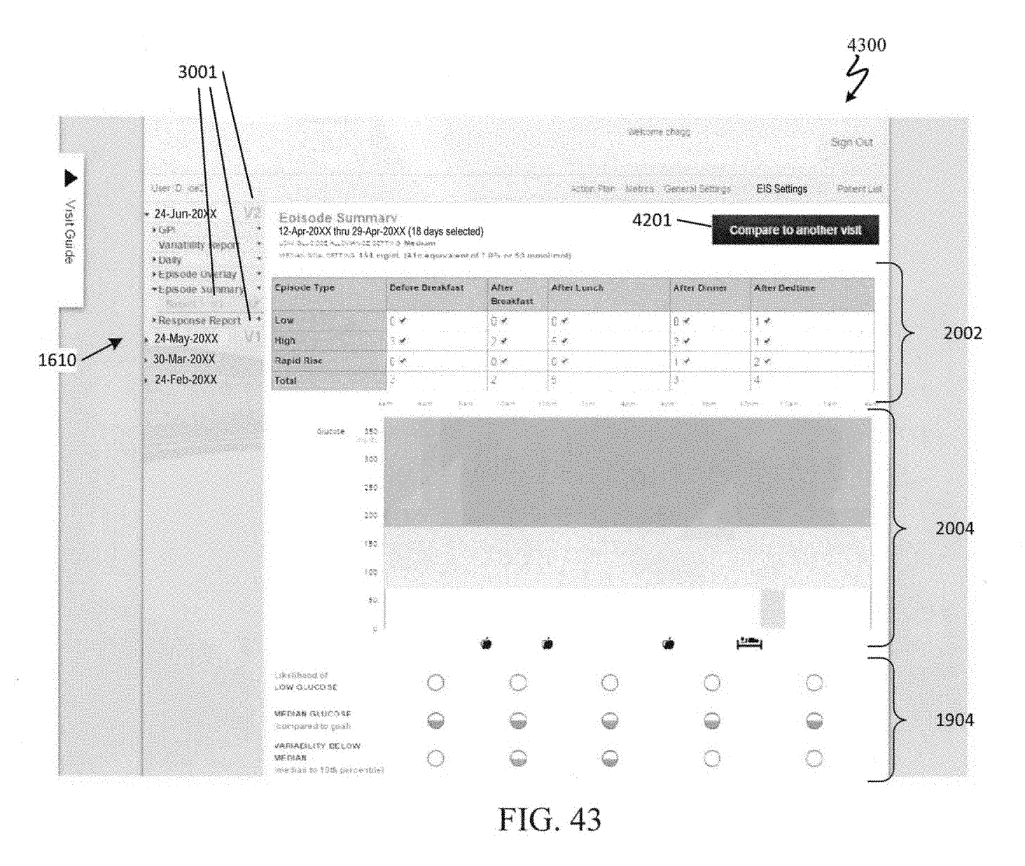

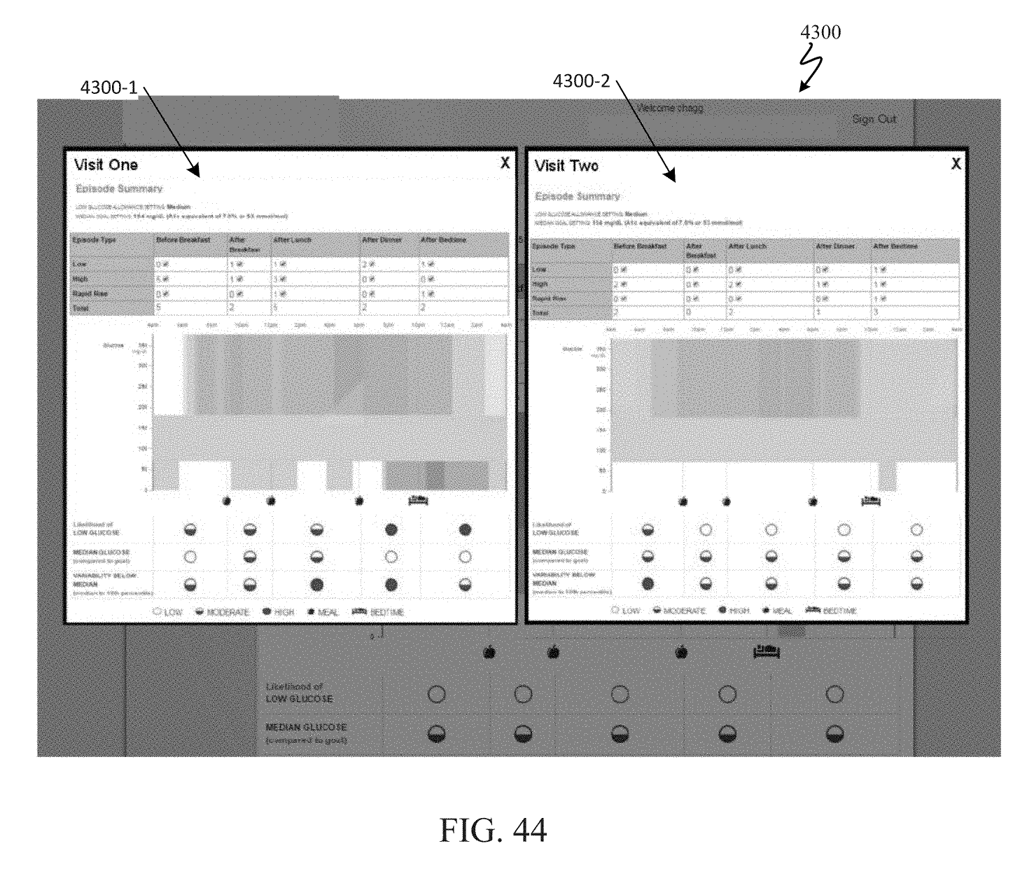

Referring back to FIG. 41, the next suggested step within expanded menu 3902 is to review the episode summary report with the patient and the link to the same is provided (“Episode Summary Report”). FIG. 43 depicts another example embodiment of an episode summary report 4300, similar to the embodiment described with respect to FIG. 20, including an episode summary table 2002, a modal day graph 2004, and region 1904. Again, the indicators 3001 in navigation bar 1610 identify which sessions and reports correspond to which visits. Selection of field 4201 can generate a drop down menu similar to that shown in FIG. 42B, where another episode summary report can be selected for comparison purposes. FIG. 44 depicts another example embodiment where pop-up windows are displayed for comparison of an episode summary report 4300-1 from Visit One with episode summary report 4300-2 from Visit Two.

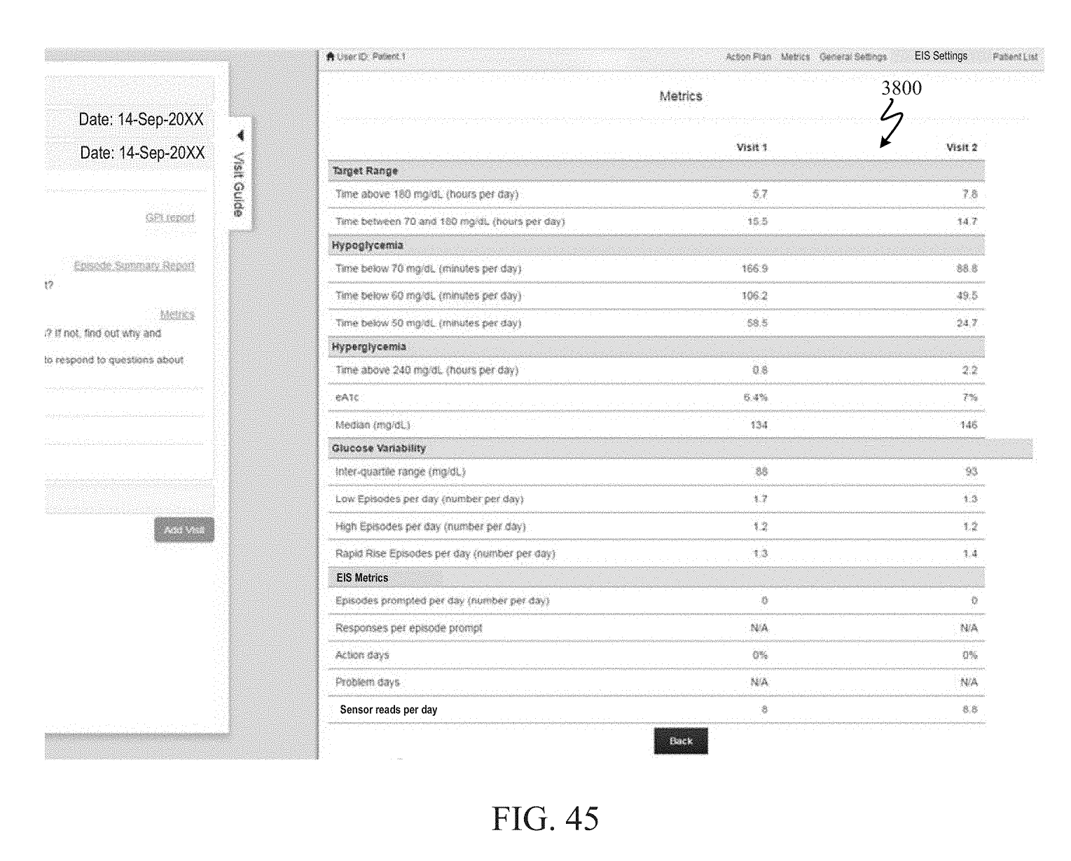

Again referring back to FIG. 41, the next suggested step within expanded menu 3902 is to review the patient's glucose control metrics and a link to the same is provided (“Metrics Control”). Selection of that link will cause the display of the metrics display screen 3800 of FIG. 45, where the metrics from Visit One are displayed alongside the metrics from Visit Two in adjacent columns for easy comparison. Note that if more than two visits exist then a column of metrics can be displayed for each visit.

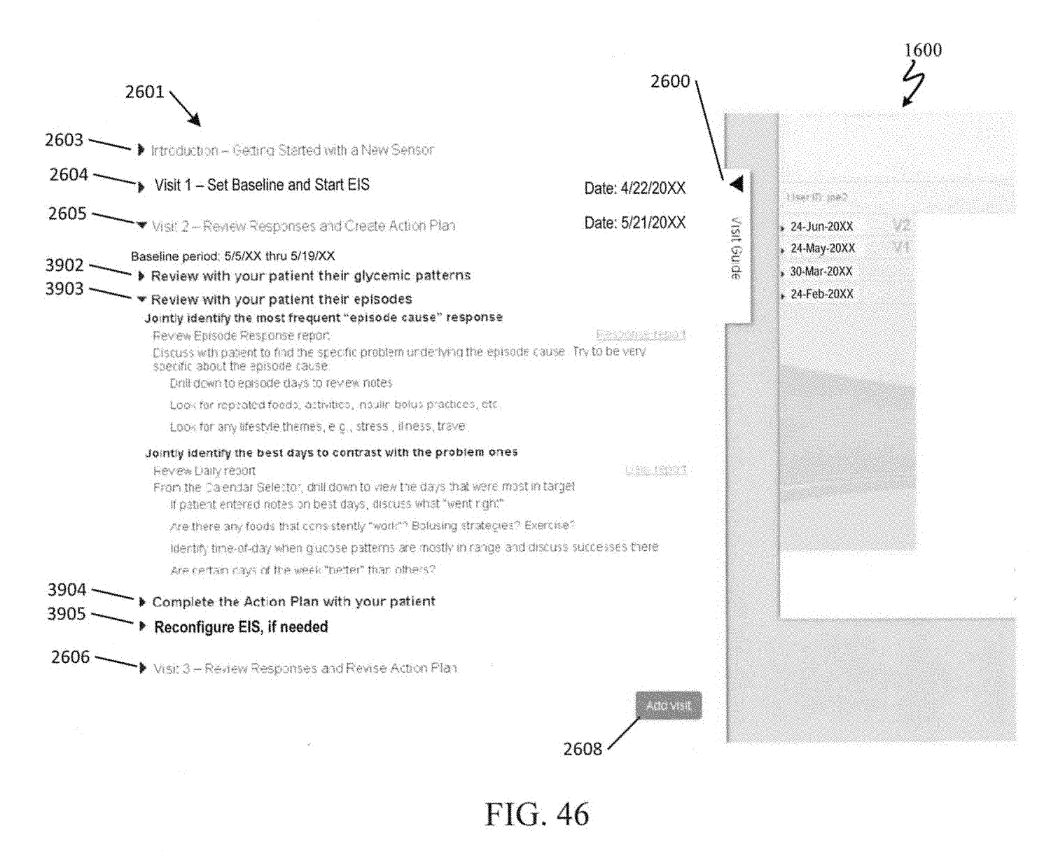

FIG. 46 depicts visit guide menu 2601 after expansion of the next selectable field 3903, which suggests that the HCP the detected episodes with the patient and attempt to jointly identify the most frequent episode cause response. The first step underneath field 3903 invites the HCP and patient to review the episode response report and a link to the same is provided (“Response report”). The goal of review of the episode response report is to identify the specific cause underlying occurrence of the episode and the HCP can consider whether there is a link between particular days, foods, activities, insulin bolus practices, lifestyle themes (e.g., stress, illness, travel), or others to help make that identification.

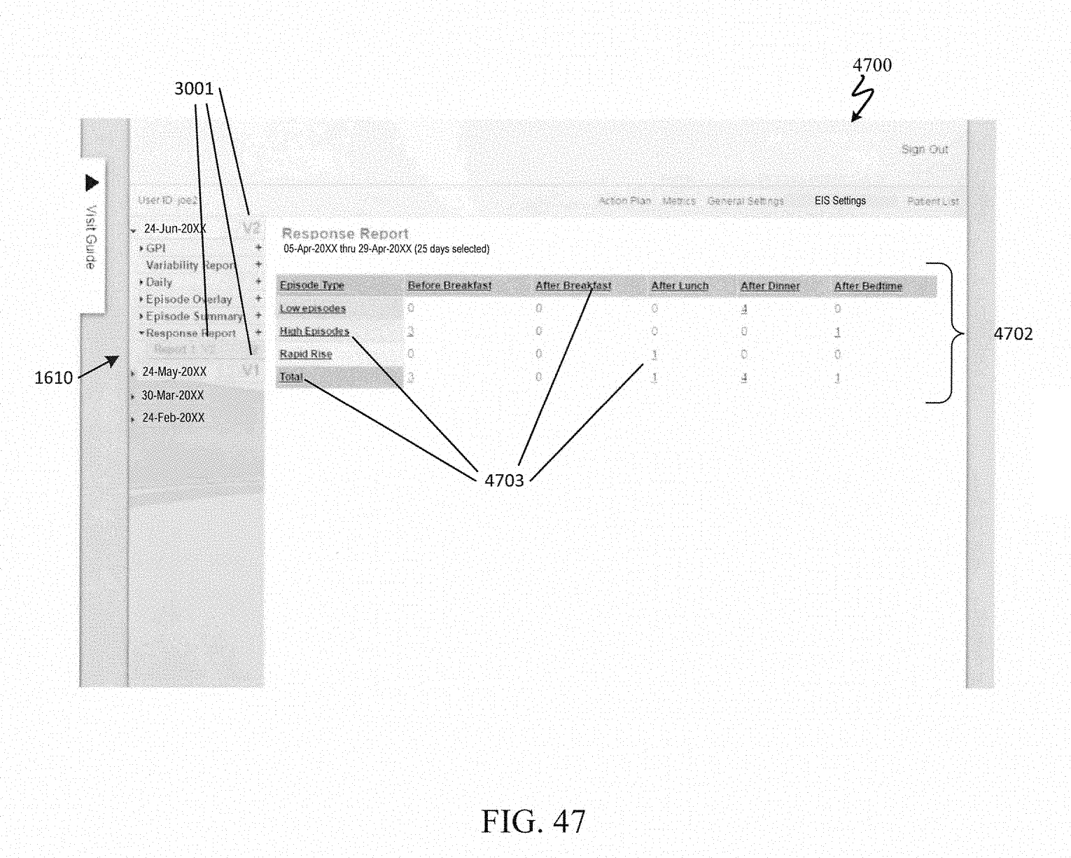

Selection of the response report link causes the display of an example embodiment of a response report screen 4700 as depicted in FIG. 47. This embodiment is a variation of the response report embodiment described with respect to FIGS. 22A-B. When first displayed, response report screen 4700 includes a response summary table 4702 where episode types are arranged vertically (e.g., along a Y axis) and time periods of the day are arranged horizontally (e.g., along an X axis). Table 4702 is populated with the number of instances where a particular episode type occurred during a particular time period. Each of the episode types, time periods, corresponding numerical entries, and totals are displayed as selectable fields 4703, and selection of a field 4703 will cause additional information about the selected episodes to be retrieved and made displayable within report 4700.

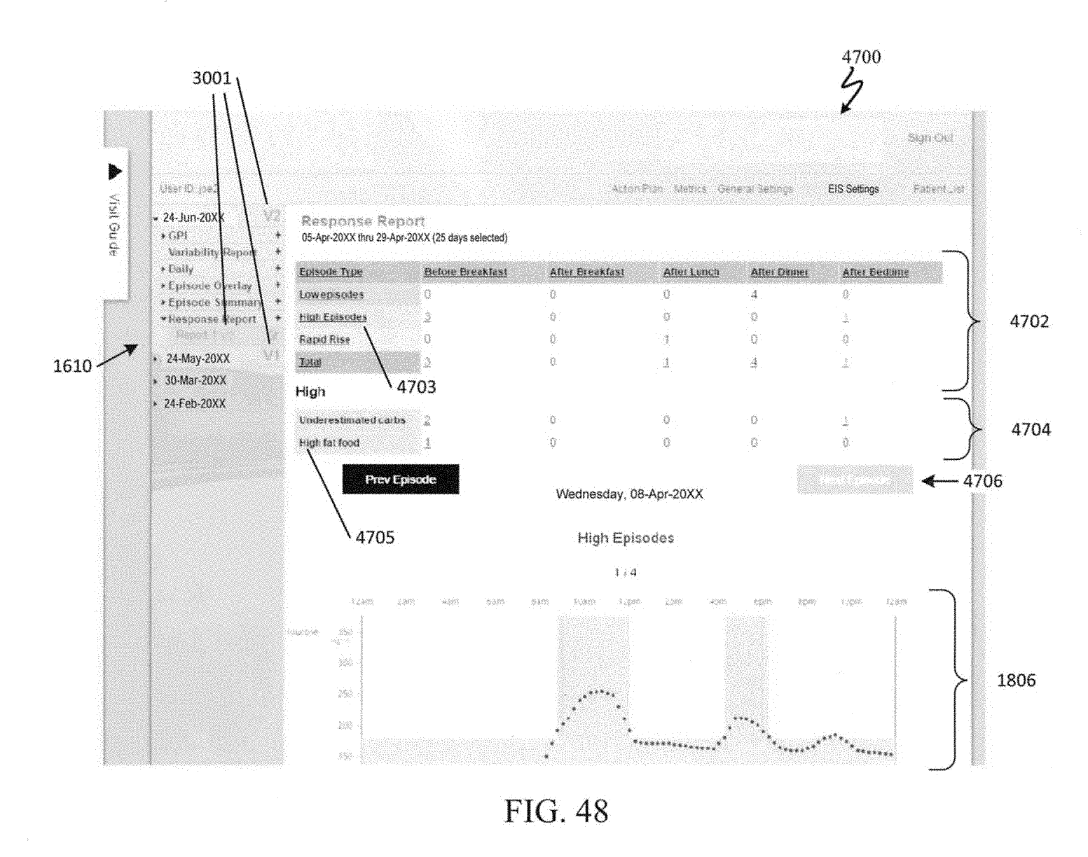

For example, selection of field 4703 corresponding to the episode type “High Episodes” causes additional regions 4704 and 1806 to be displayed as depicted in FIG. 48. Region 4704 contains a table that lists each cause that was selected by the patient in response to detected high glucose episodes. Those causes in this embodiment are “Underestimated carbs” and “High fat food” and they are arranged vertically in a first column of the table. The adjacent columns indicate how many times each particular cause was identified and during which time period.

Region 1806 is a daily graph (as previously described herein) that contains the glycemic data collected around the time when a first one of the high episodes was detected. Selectable fields 4706 allow the user to toggle daily graph 1806 from one high episode to another within the group of high episodes selected (which includes four episodes in this example).

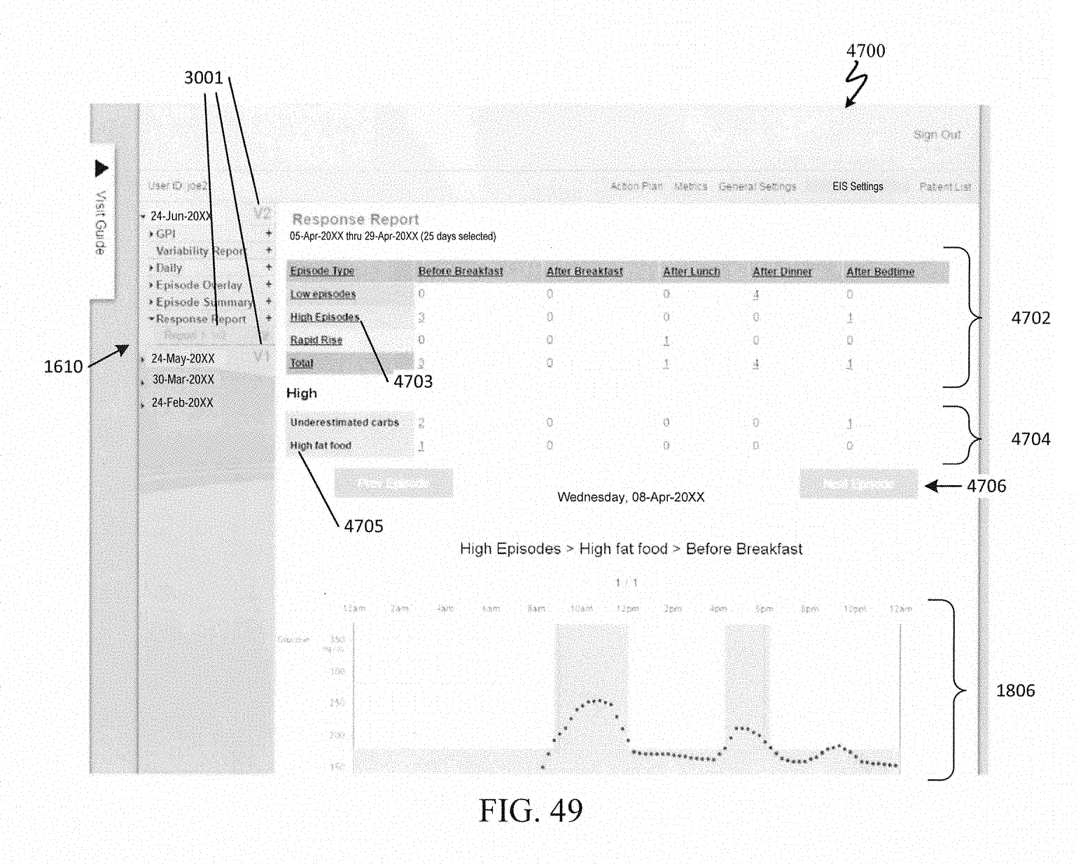

Each of the potential causes and numerical entries within the table of region 4704 is a selectable field 4705 were selection of that field will cause the corresponding data to be made accessible in daily graph 1806. Each of the FIG. 49 depicts another example embodiment of response report 4700 after selection of field 4705 (the number “1”) corresponding to the one occurrence of the “High fat food” cause “Before Breakfast.” Here, daily graph 1806 has been updated to show the data from that one episode occurrence.

By way of another example, if “Before breakfast” is selected, then all days with any episode that starts during the “Before breakfast” time period are available for navigation. As yet another example, if the count metric in the cell aligned with the “Underestimated carbs” cause and “After bedtime” time period is selected, then all days that satisfy these conditions are available for navigation. An alternative embodiment of this functionality is to display a predetermined subset of this information. For example, the report could display only the most frequent response and associated episode and time-of-day. Referring to FIG. 49 this could be (ignoring the 4 low episodes after dinner for illustrative purposes) “Underestimated carbs: 2 high episodes before breakfast.” In another embodiment, the report could display only the most frequent response for all times of day: “Underestimated carbs: 3 high episodes.” Many other subsets are contemplated, all of which can also be sent as a notification to the mobile access platform periodically to inform the patient directly without requiring them to interact on their own initiative with any version of the EIS.

The processor operating the EIS monitors for selection of any of the fields 4703 and 4705 and upon detecting a user selection will cause the response report 4700 to be updated on the display, through updates to table 4704 and/or daily graph 1806. Referring back to FIG. 46, expanded field 3903 next suggests that the HCP utilize the daily report to contrast days during which performance is good with days during which performance is relatively bad and a link to the “Daily report” function is provided, where the data for each day within the baseline period can be viewed. In this fashion, the HCP and patient can jointly investigate the existence of causal trends to any and all of the episodes detected in the baseline data.

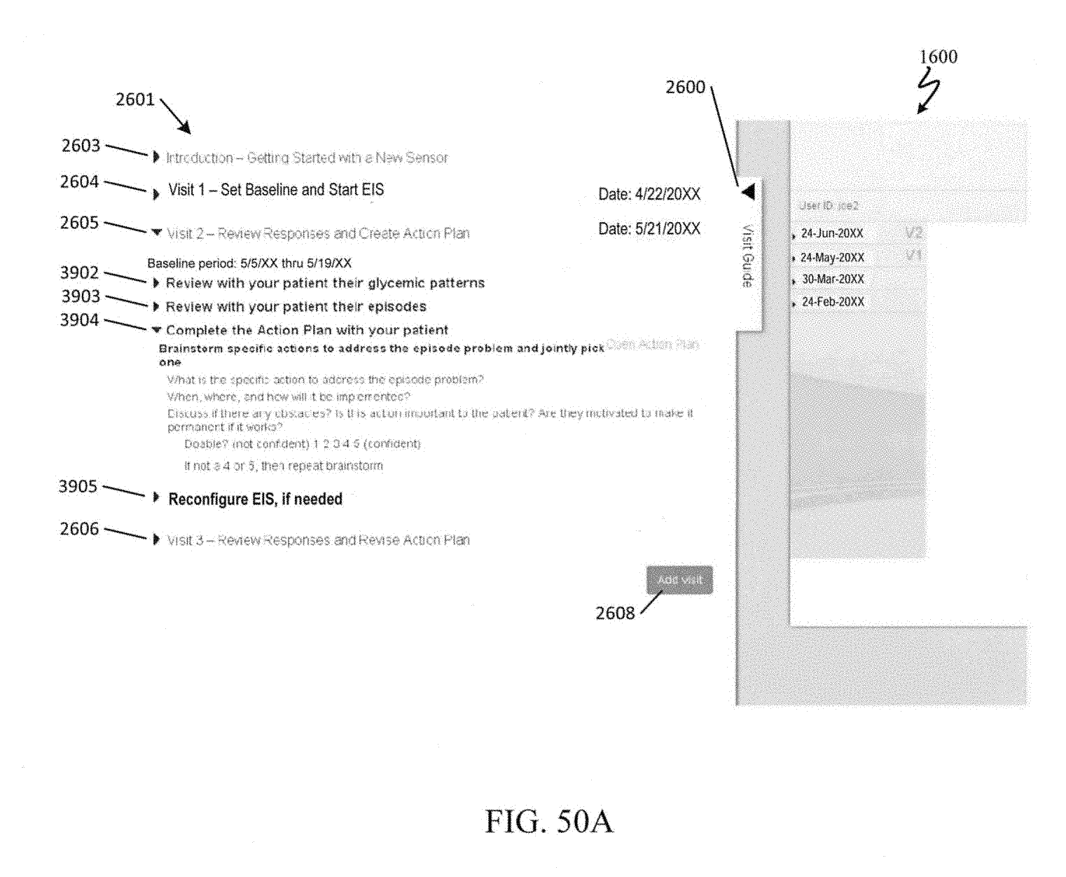

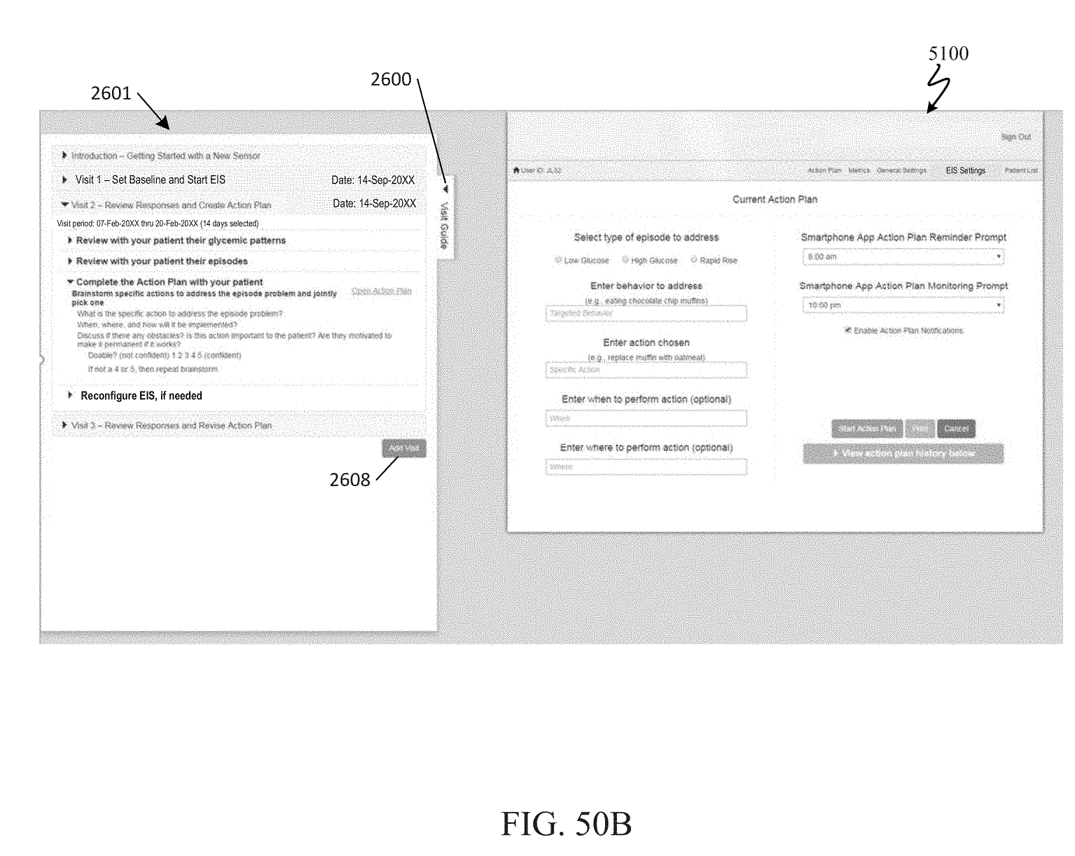

FIG. 50A depicts visit guide menu 2601 after expansion of the next selectable field 3904, which suggests that the HCP complete an action plan with the patient and includes a link to the same (“Open Action Plan”). The action plan contains an agreed-upon set of one or more actions that the patient can undertake to address the causes of the episode occurrence. The action plan is intended to help encourage the patient to follow through with the actions that are decided on with the HCP.

For example, in certain embodiments of the EIS, starting with Visit Two and for subsequent visits, the EIS provides a script and control mechanism for creating and updating the action plan for the patient. In addition, the action plan control mechanism can also be available outside the visit guide, accessible from tool bar 2620 (see FIG. 26) where action plans can be created or updated at any time. FIG. 50B depicts an example embodiment of the screen display after selection of the action plan link in the visit guide menu 2601. The action plan control interface 5100 is displayed to the side of the visit guide menu 2601.

FIG. 51A depicts the action plan control interface 5100 in greater detail. The action plan control interface 5100 includes fields that are used to identify the particular episode type and behavior to address, and to collect information about the particular action that the HCP and patient decide on. This information can then be transmitted to the reader device 120 (e.g., a smart phone storing the downloaded EIS app) and is available for the patient to view, selectable from the menu on reader device 120.

Field of 5102 allows the user to select the type of episode that is being addressed by the chosen action, in this embodiment, those episode types are low glucose, high glucose, and rapid rise. Field 5104 allows the user to enter the particular behavior that is being addressed by the chosen action. In this embodiment, field 5104 is a free text entry field that allows the user to enter any behavioral description desired. When this action plan is saved, the entered problem behavior is added to the list of potential causes for the type of episode that is selected and this list is transmitted to reader device 120 and appears on the response pick list 1298 for potential causes of the selected episode type.

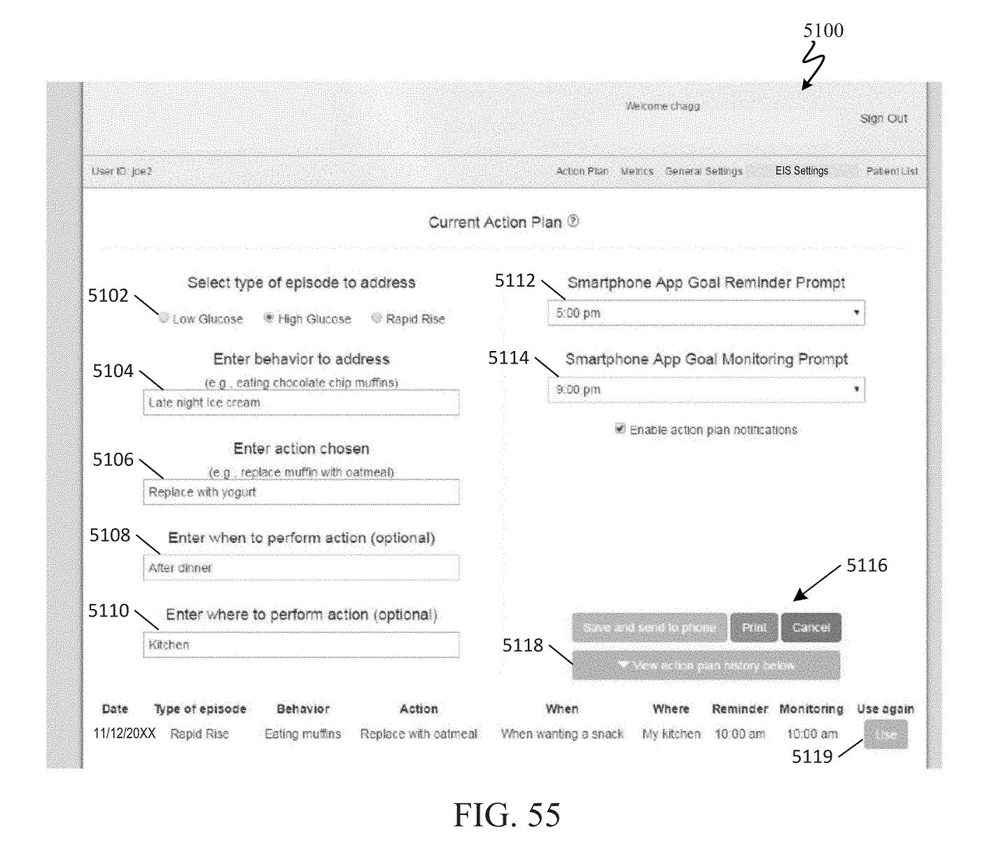

Field 5106 allows the user to enter a description of the action chosen to address the behavior leading to occurrence of the particular episode type. Field 5108 allows the user to optionally enter a description of when the chosen action is to be performed and field of 5110 allows the user to optionally enter a description of where the chosen action is to be performed. Fields 5106-5110 are also free text entry fields that provide the user with additional freedom in describing the attributes of the action. Field 5112 allows the user to set a time at which reader device 120 will issue or display an action plan goal reminder prompt and field 5114 allows the user to set a time at which reader device 120 will issue or display an action plan goal monitoring prompt. The user can use fields 5116 to save and submit the entered information and thus start an action plan (see “Start Action Plan” field), print the entered information (see “Print” field), or cancel entry of the action plan (see “Cancel” field) if desired. An expandable field 5118 is present at bottom and selection of that field causes display of an action plan history with a description of the settings for that prior action plan (see FIG. 55).

Upon saving and submitting the entered action plan information by selecting the Start Action Plan field, that information is transmitted to reader device 120. The patient can access the action plan on a reader device 120, for example by activating a drop down menu 5122 on an example embodiment of a reader device home screen 5120 as depicted in FIG. 51B. Selection of the action plan from menu 5122 can cause the display of screen 5130 including the specific information entered with respect to the action plan, such as the targeted cause, the specific action, and when and where the action should be taken.

At the time set for display of the action plan goal reminder prompt, that prompt 5142 can be displayed on any screen (e.g., screen 5140) within the EIS app as depicted in FIG. 51D or on the patient's lock screen 5150 of reader device 120 as depicted in FIG. 51E. Similarly, at the time set for display of the action plan goal monitoring prompt, that prompt 5162 can be displayed (or can pop-up) on any screen (e.g., screen 5160) within the EIS app as depicted in FIG. 51F or on the patient's lock screen 5150 of reader device 120 as depicted in FIG. 51G. The text presented in prompt 5142 and 5162 is the “action” text entered in field 5106. The goal monitoring prompt 5162 asks the patient whether the goal for the day was achieved and includes one or more selectable fields where the answer can be provided. This information is stored with the other data collected by the EIS app and uploaded to the server where these responses can be used to drive another metric and/or be made available for consultation with the HCP. With smartphone readers, reminders 5142 and monitoring prompts 5162 can be implemented using the standard notification mechanism such as that available on the Android and iOS operating systems.

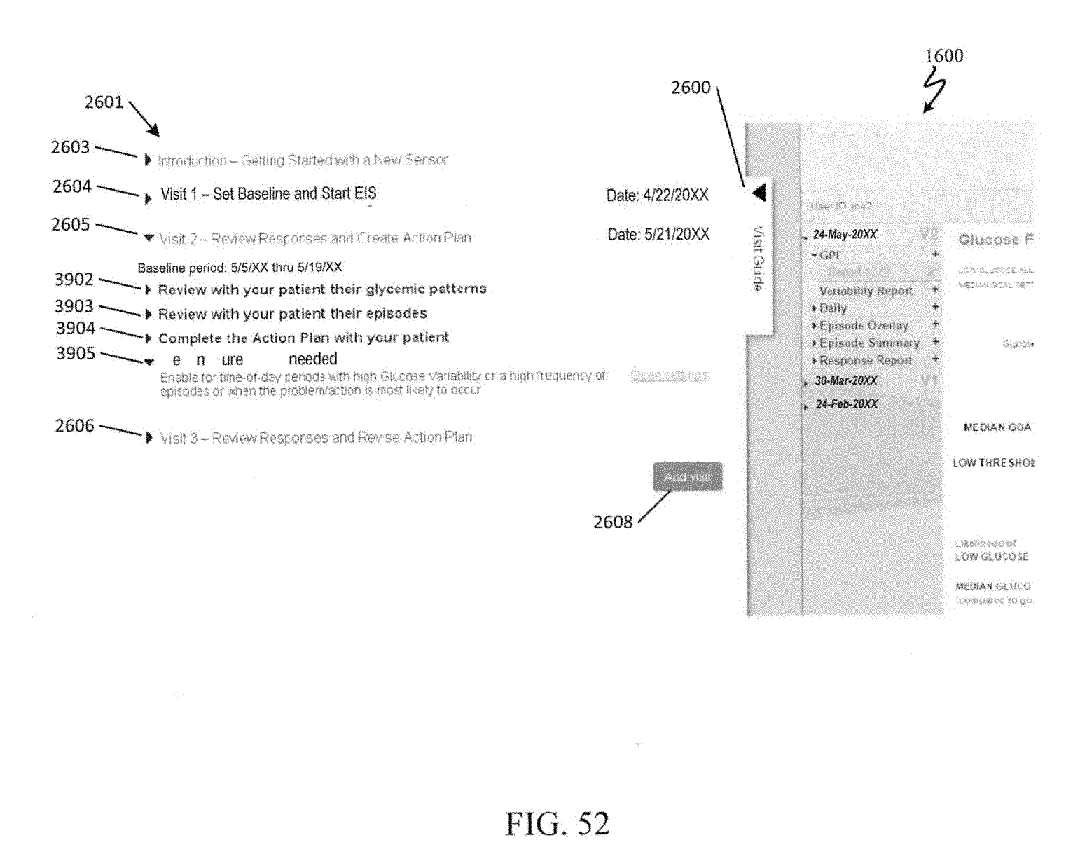

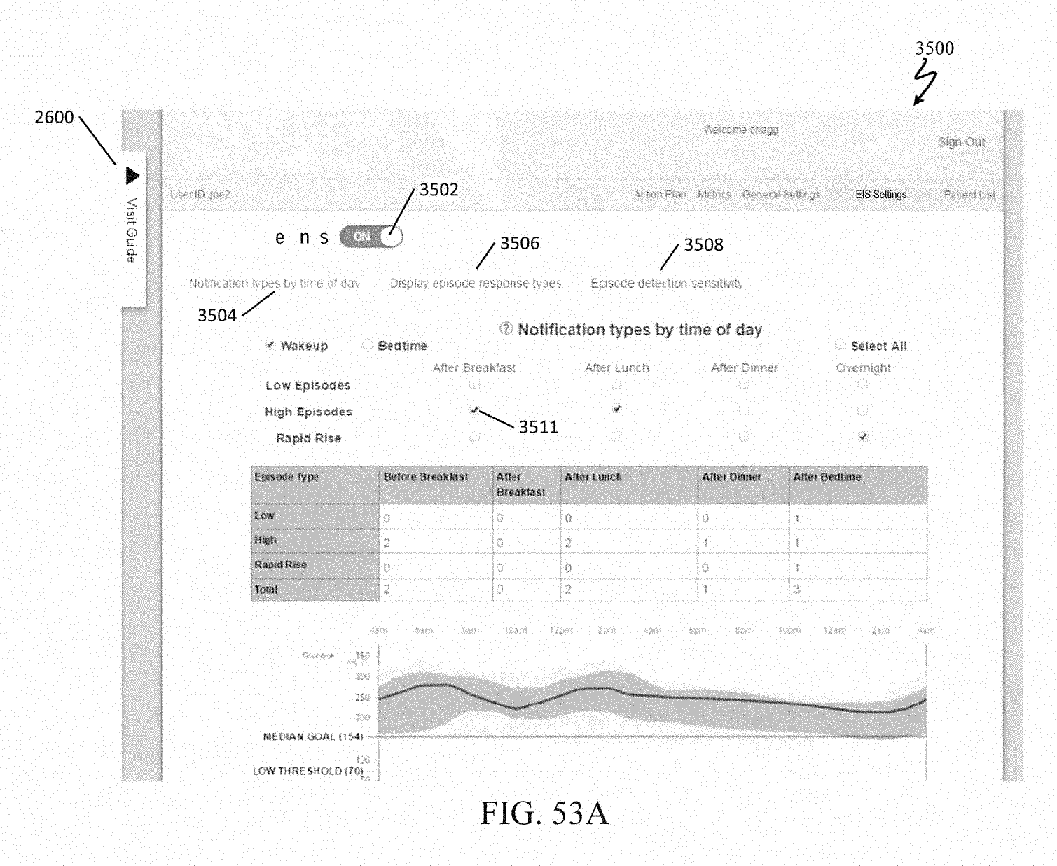

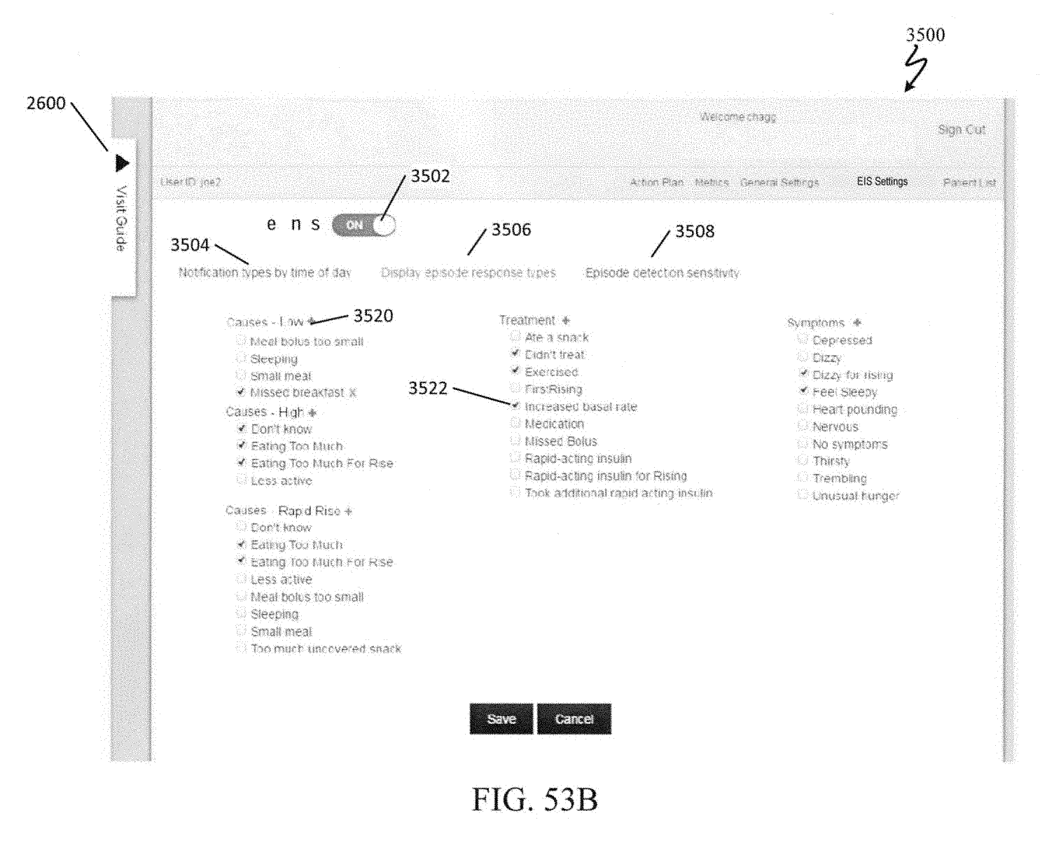

FIG. 52 depicts visit guide menu 2601 after expansion of the next selectable field 3905, which suggests that the HCP reconfigure the EIS settings to make any necessary adjustments, and includes a link to the same (“Open settings”). Selection of that link will bring the user to settings screen 3500. FIG. 53A depicts another example of tab 3504 of settings screen 3500 where a subset of notification types by time-of-day 3511 have been selected corresponding to those episode types and times of day that are most problematic for the patient. FIG. 53B depicts another example of tab 3506 of settings screen 3500 where various fields 3522 for subsets of causes, treatments, and symptoms have been selected in a manner customize to patient. Upon reconfiguring the EIS settings, Visit Two can be concluded.

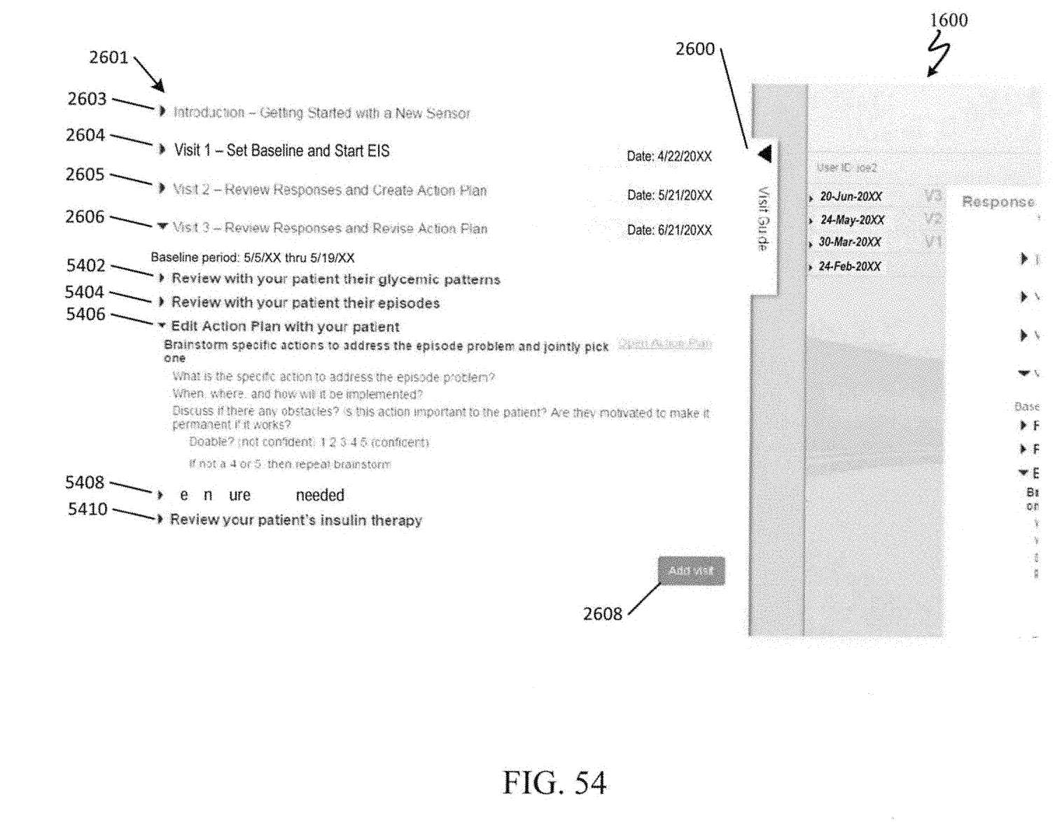

FIG. 54 depicts an example embodiment of visit guide menu 2601 where expandable field 2606 corresponding to a third visit (“Visit Three”) has been expanded. Within field 2606 are a series of suggested actions to take during the third visit, each corresponding to its own expandable field. Field 5402 suggests the HCP review the patient's glycemic patterns with the patient in a manner similar to that previously described herein. Field 5404 suggests the HCP review the patient's episodes with the patient in a manner similar to that previously described herein.

Field 5406 a shown here in FIG. 54 as being expanded. Field 5406 suggests that the HCP edit the action plan with the patient. This can entail editing the action to address the problem behavior identified during the second visit and/or arriving at new specific actions to address newly identified episodic problems. A link to the action plan control interface 5100 is provided (“Open Action Plan”).

FIG. 55 depicts another example of control interface 5100 where the various fields have been completed to address a high glucose episode type and problem behavior referred as “Late night ice cream.” The action chosen to address that behavior it is referred to as “Replace with yogurt” and is to be performed “After dinner” in the “Kitchen.” Field 5118 at bottom has been expanded to cause the display of an action plan history with a description of the settings for that prior action plan. Those settings can be used again by selection of field 5119, which will cause the corresponding settings to be populated in the various fields 5102-5114 where they can be modified or accepted as-is.

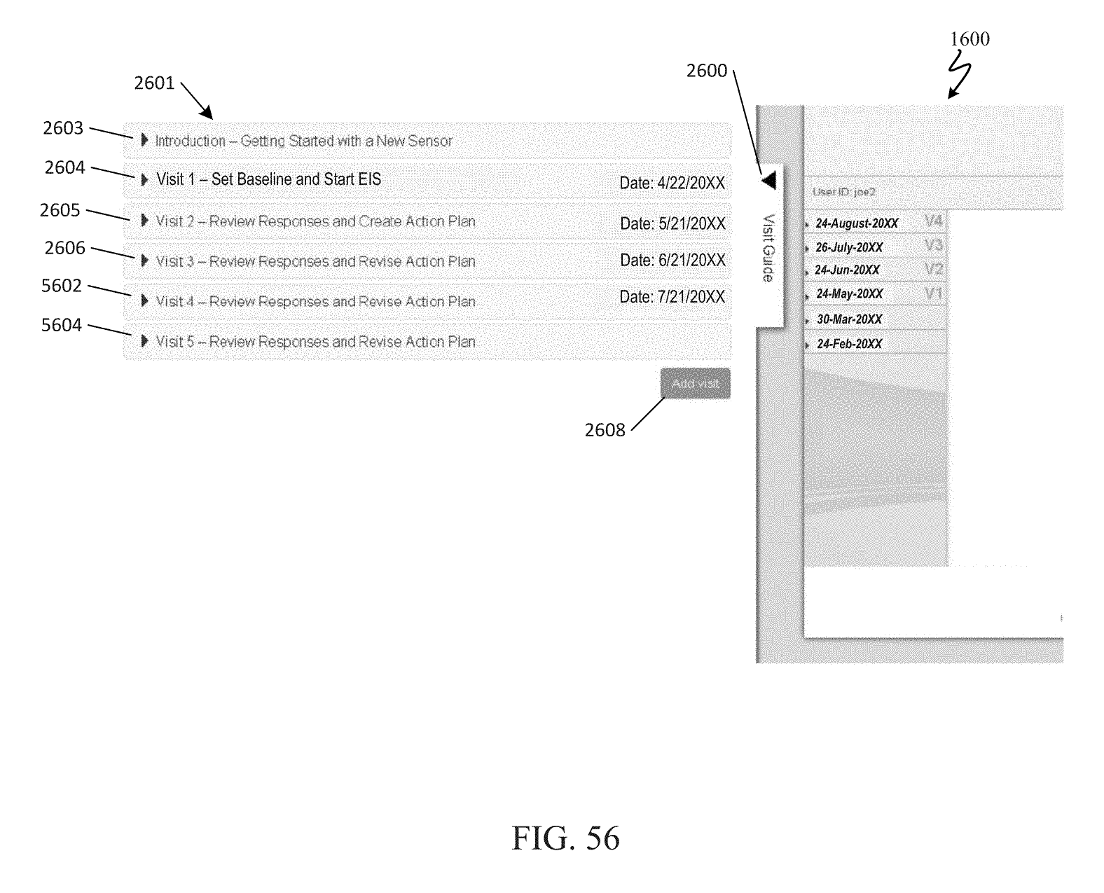

In this manner, the HCP and patient can continually identify episodic problems and their corresponding behaviors, identify one or more actions to address them, create an action plan to facilitate modifying the patient's behavior, evaluate the degree to which the actions are actually performed, and update or revise the action plan as needed. This process can proceed across subsequent visits. FIG. 56 depicts an example of visit guide menu 2601 where two additional visits have been added using the add visit field 2608. These visits have corresponding expandable fields 5602 (for Visit Four) and 5604 (for Visit Five). Expandable fields 5602 and 5604 can contain similar text and links as that contained within expandable fields 2606 corresponding to Visit Three. In this manner any number of additional visits can be added and tracked. Any reports and sessions generated during each visit will be assigned a corresponding indicator 3001 (e.g., V4, V5, etc.).

Example Embodiments of Episode Investigative Software (EIS) with Multiple Patient Scheduling Features

Also described herein are example embodiments of the EIS that improve the efficiency for HCPs or clinicians to confer with multiple patients throughout the course of a day or week. These example embodiments allow the HCP to more efficiently comprehend the HCP's schedule for a particular time period (e.g., a day, a week, or a month) and the relative position of each patient in his or her own treatment process, and allow the HCP to access information representative of the improvement (or lack thereof) in treatment of each individual patient's glycemic patterns.

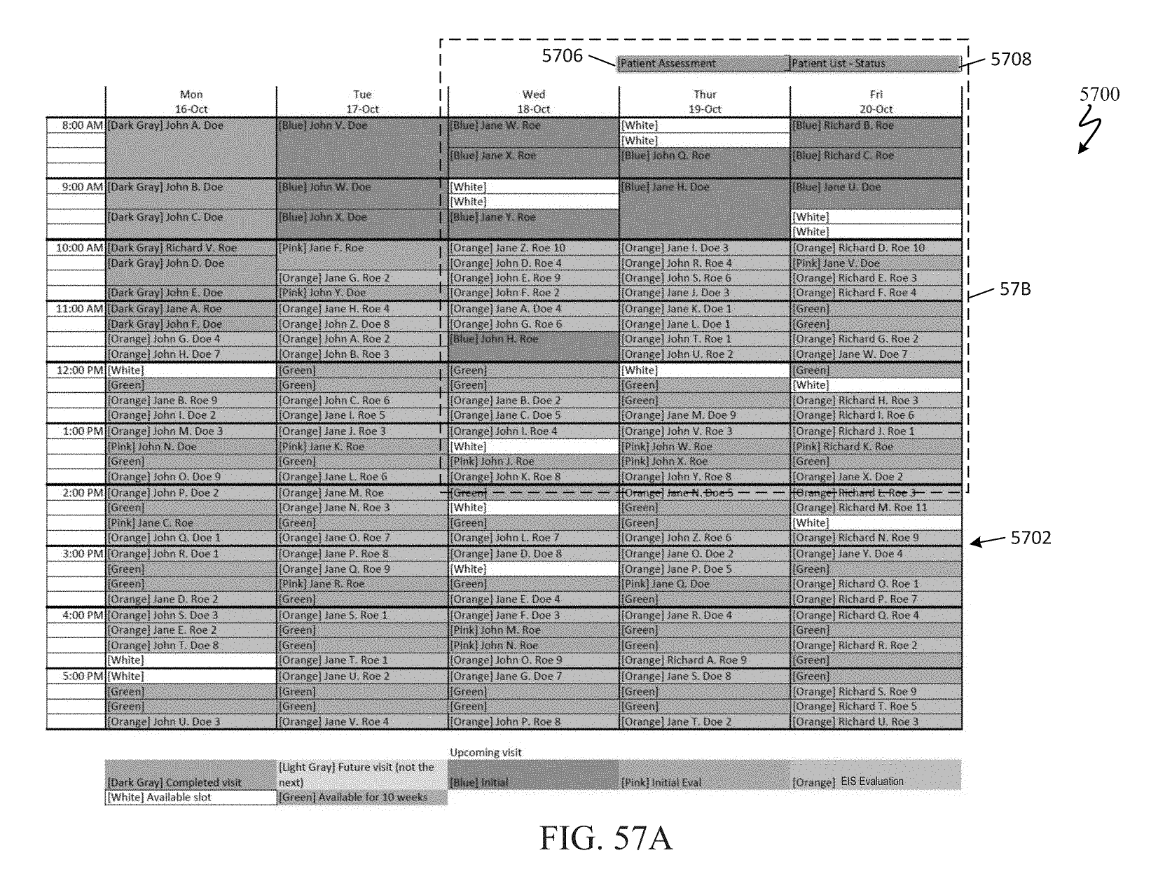

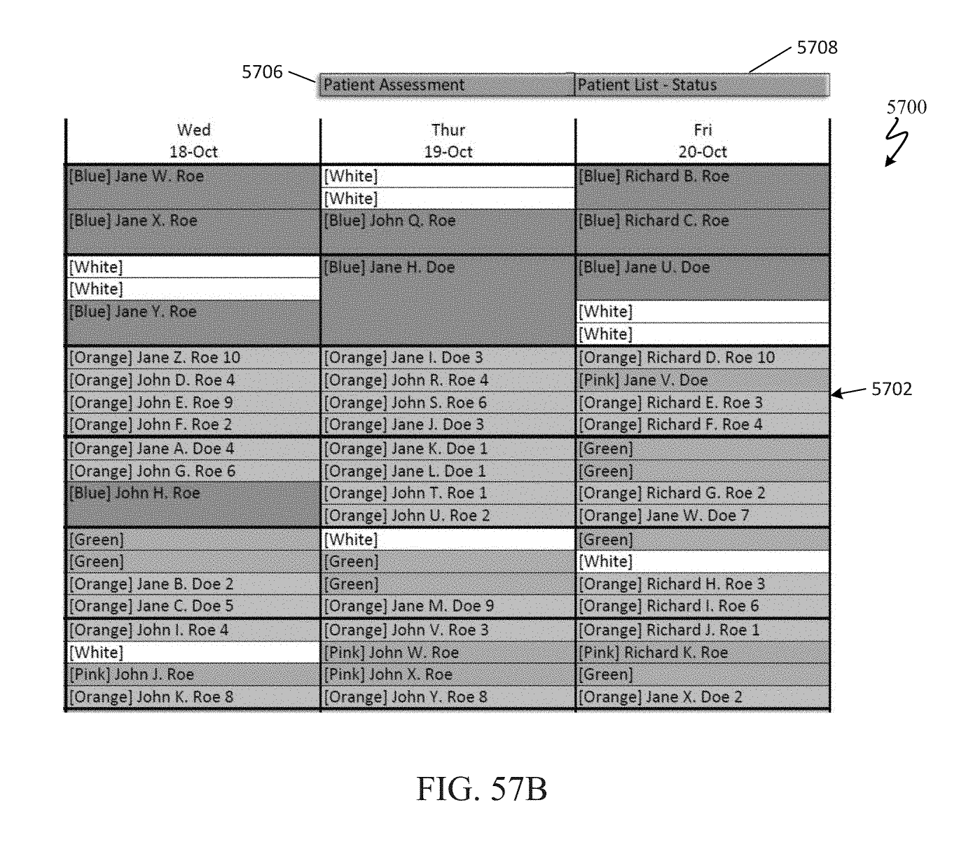

FIGS. 57A-60 depict example embodiments of GUI screens for the EIS with this efficient scheduling functionality and structure. Like other embodiments described herein, these embodiments are displayable on any computing device executing or displaying the EIS, such as through a software application resident on the computing device or through an internet browser. These GUI screens can be generated by a server for display on the user's computing device (such as with an internet browser), or they can be generated directly by the user's computing device (such as with a resident software application), in both cases using software instructions stored on non-transitory memory of the respective device (server or computing device) and executed by processing circuitry (e.g., one or more processors) of the respective device (server or computing device).

FIGS. 57A-60 depict example embodiments that are intended for (but not limited to) display with the dual access platform described above (e.g., on a large screen computing device using an internet browser or web application). However, the embodiments of FIGS. 57A-60 can also be displayed on the mobile access platform described above (e.g., through an app operating on a smart phone). Information or settings entered into the EIS via the dual access platform (or information processed or determined by the dual access platform) can be downloaded to the mobile access platform through the network as needed, and vice-versa.

FIG. 57A depicts an example embodiment of a scheduling interface screen 5700, with portion 57B shown in greater detail in FIG. 57B. Scheduling interface 5700 can be integrated with the visit guide and/or action plan embodiments described elsewhere herein. In some embodiments, scheduling interface 5700 can be accessed from the EIS, such as a default screen or home screen 1600 (e.g., described with respect to FIG. 16), where the user can optionally access visit guide functionality (e.g., visit guide menu 2601) and/or action plan functionality (e.g., action plan control interface 5100).

In some embodiments, visit guide menu 2601 can be accessed directly from scheduling interface 5700 via a user selectable field (not shown). In other embodiments, scheduling interface 5700 can be a distinct software application without a direct link to visit guide menu 2601 as described herein. In these embodiments, some or all of the functionality of visit guide menu 2601 can be integrated directly into scheduling interface 5700. The embodiments described with respect to FIGS. 57A-60 are discussed as embodiments of the distinct implementation.

In FIGS. 57A-57B, scheduling interface 5700 utilizes a similar approach to that described with respect to the visit guide (see, e.g., visit guide menu 2601 of FIG. 27A), where each patient visit is classified into at least one of multiple different types. For example, those types can include, but are not limited to: an Initial Visit, an Initial Evaluation Visit; and/or one or more Subsequent Evaluation Visits. At an Initial Visit the patient can be introduced to sensor control device 102 and its manner of use (e.g., such as the Introductory Visit described with respect to FIG. 28). At an Initial Evaluation Visit, the patient has worn the sensor for the first time for, e.g., one to two weeks and the HCP will make an initial assessment of the patient's glucose patterns and establish a baseline (e.g., such as Visit 1 described with respect to FIGS. 29-38). The HCP can then enable the EIS features on the mobile platform, e.g., the patient's phone. In some cases, if the patient has already been wearing a sensor control device 102 at the beginning of the program, then the Initial Evaluation Visit may be scheduled instead of the Initial Visit. The Initial Visit and Initial Evaluation Visits are preferably in-person visits at the HCP's office and, e.g., may last from 15 minutes to an hour.

At an EIS Evaluation Visit the HCP can review the EIS reports with the patient and develop a course of action with the patient (e.g., such as in Visits 2, 3, 4, and 5 and the creation and management of the Action Plan, which are described with respect to FIGS. 39-56). One or more EIS Evaluation Visits should occur, and in some cases, there may be 5, 10, or more of these visits on a regular interval, such as weekly or bi-weekly. The EIS Evaluation Visits can be in-person or remote. Preferably, the patient will teleconference, videoconference, and/or web-conference with the HCP's office or clinic, while viewing the EIS reports along with the HCP on the display of a computing device (e.g., laptop, tablet, mobile phone, etc.). The patient can utilize the mobile platform for this purpose, and can view the same displays as the HCP or the HCP can selectively choose which reports to display to the patient, such as with a sharing function (e.g., a “share” option or button) on each report that communicates the relevant report data to the patient's computing device where it is rendered for display to the patient.

Scheduling interface 5700 (FIG. 57) can include a calendar 5702 that can be configured to display reserved appointments for various different patients (e.g., John A. Doe, John B. Doe, etc.) in a grid-type format, with multiple columns and rows. Here, calendar 5702 is a five-day workweek calendar with days arranged in columns and timeslots arranged in rows. Other formats for calendar 5702 can also be used, such as a seven day week, a single day, a full month, two consecutive weeks (5 or 7 days), and so forth.

Scheduling interface 5700 can include one or more features that facilitate and the HCP's scheduling activities and increase the efficiency at which the HCP can handle or work through the scheduled patient workload. Scheduling interface 5700 can be similar to scheduling programs known to those of ordinary skill in the art, with the addition of these features. Population of calendar 5702 can be accomplished by the HCP or an administrator associated with the HCP. When entering an appointment, the type of appointment can be selected (e.g., from a drop down menu) and can default to a standard length of time based on the appointment type (e.g., 60 minutes for an Initial Visit, 15 minutes for an Initial Evaluation Visit or EIS Evaluation Visit, etc.) and these time durations can be customized as desired.

Each time slot displayed on calendar 5702 can be visually coded to indicate one or more aspects about the time slot, such as whether a patient has an appointment scheduled during that slot and, if so, the status of the appointment and/or the type of appointment. Visual coding helps the HCP quickly and efficient determine the context of the upcoming visit to aid in preparation. Any type of visual coding can be used, such as one or more of the following: colors, degrees of grayscale, numbers, text or textual codes, and others. In this example embodiment, scheduling interface 5700 utilizes a combination of colors, grayscale, numbers, and text. Here, for example, time slots available for a particular day can be shown in white, while slots available for a number of weeks into the future (e.g., 2, 5, 10, etc.) can be shown in green. If a time slot has been reserved for a patient then the name of the patient can be indicated in text and the color can be coded based on the visit type, for example, blue for an Initial Visit, pink for an Initial Evaluation Visit, and orange for an EIS Evaluation Visit.

Functionality of visit guide menu 2601 can be directly utilized in embodiments of scheduling interface 5700. For example, help text associated with the visit type can be displayed when an element of scheduling page 5700 is selected, where the visit help text (e.g., such as that associated with, or, to name a few) is associated with the type of visit for the time slot (e.g., field 2603 of FIG. 28 for an Initial Visit, fields 2902-2906 of FIG. 31B for an Initial Visit, field 3904 of FIG. 50A for an Initial Evaluation Visit, fields 3902-3905 of FIG. 39 or fields 5402-5406 of FIG. 54 for an EIS Evaluation Visit, etc.).

In addition, for EIS Evaluation Visits, a number can be presented adjacent the patient's name that indicates where in the sequence of EIS Evaluation the scheduled appointment will fall. For example, a one (1) indicates the scheduled visit will be the patient's first visit, a two (2) indicates the scheduled visit will be the patient's second visit, and so forth. Alternatively, the number can indicate how many EIS Evaluation Visits the patient has had not including the appointment shown. If the EIS Evaluation has a set limit, such as ten visits, then that number limit can indicate the last visit, or a different visual coding (e.g., color) can be used. In this example only the upcoming (or next) visit is color coded and visits after the next scheduled visit are shown in light grey. Completed visits can be shown in dark grey.

In some embodiments, the color of the time slot can be shaded to indicate the position in the sequence of EIS Evaluations that the scheduled visit falls (this can be in addition to, or as an alternative to, the use of the number). For example, progressively darker shades can indicate EIS Evaluation Visits progressively further into the sequence. Alternatively, the shading can be indicative of a metric or combination of metrics, such as a metric indicative of the patient's progress towards his or her goal. In some example embodiments, the shading is correlated with eA1c of the patient or standard deviation of the patient's glucose values, so the HCP can quickly and efficiently determine how challenging the next visit may be and whether additional preparation is needed. Alternatively, the actual metric value may be displayed in each time slot of interface 5700. The metric or combination of metrics is available to scheduling interface 5700 since the EIS has access to the patient's analyte data and related information (e.g., goals, targets, etc.) as described elsewhere herein.

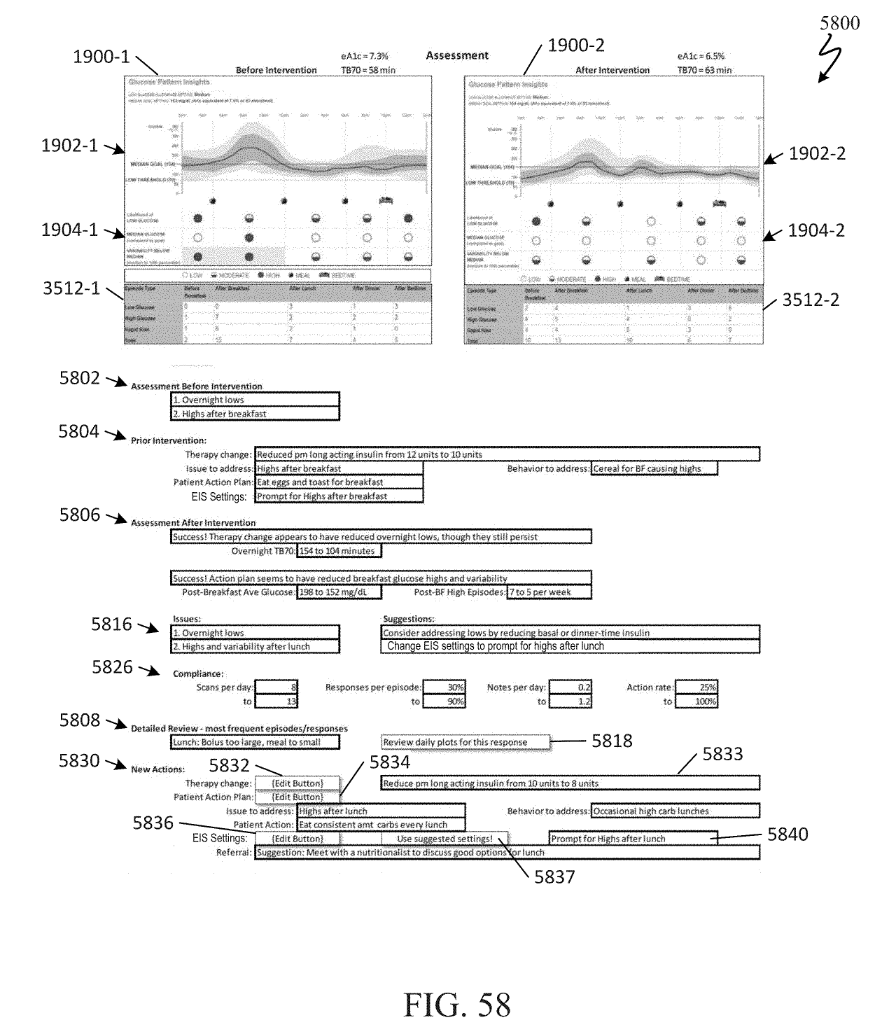

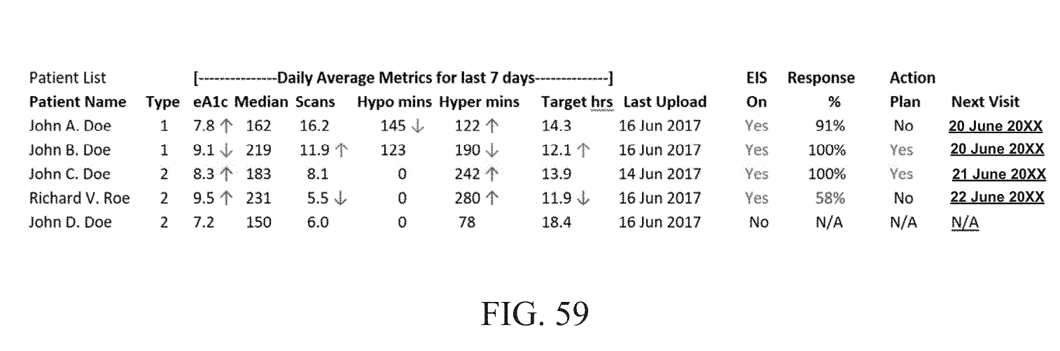

Scheduling interface 5700 can include user selectable fields 5706 and 5708. User selection of field 5706, in this embodiment, causes the display of a patient assessment report 5800 (FIG. 58), while selection of field 5708 causes the display of a patient status list 5900 (FIG. 59). Screens 5800 and 5900 can be displayed as new screens within the software, new tabs within the software, new windows of the software, or as a pop-up window overlaid on scheduling interface 5700.

Referring first to FIG. 59, patient status list or report 5900 includes the identities of patients with, e.g., visits on the HCP's calendar for a particular upcoming time period (e.g., the next two hours, four hours, the present day, the next day, etc.) along with key status metrics for the patient, including if there is a subsequent (next) visit on calendar and, if so, when that visit is set to occur. Patient status list 5900 provides a convenient mechanism to permit the HCP to efficiently prepare for immediately upcoming visits.

FIG. 58 depicts an example embodiment of a graphical user interface screen for patient assessment report 5800, which can be displayed by selecting a name on scheduling interface 5700 or by selecting field 5706 (FIG. 57). In other embodiments, field 5706 can be selected and then the patient can be identified (e.g., by entering the name into a search tool, choosing the name from a drop-down list, etc.).

Patient assessment report 5800 includes various types of information related to the patient's glucose values, treatment, and/or actions before and after the most recent intervention (e.g., EIS Evaluation Visit) to enable the HCP to more efficiently judge the effectiveness of the last intervention. Report 5800 will be described with reference to various regions in the report containing different types of information, although the order and placement of these types of information can be changed as desired in other embodiments.

In this embodiment, the top left of report 5800 displays a first GPI Report 1900-1 (e.g., such as those described with respect to FIGS. 19A-19B and/or in incorporated US. Publ. No. 2014/0188400) with an episode summary 3512-1 (e.g., similar to that described with respect to FIG. 35B) in an adjacent location (e.g., beneath) for the patient with data collected prior to the most recent past intervention. Here, GPI Report 1900-1 can include a first region 1902-1 that displays an ambulatory glucose profile (AGP) of the user and a second region 1904-1 that displays assessments of the severity of certain conditions related to the user's diabetes. Episode summary 3512-1 can summarize the types of episodes occurring during various time regions throughout the day. Here, the time regions are divided based on typical meal times (e.g., before breakfast, after breakfast, after lunch, etc.). The top right of report 5800 displays a second GPI Report 1900-2 and episode summary 3512-2 for the patient with data collected after the most recent intervention. For an Initial Evaluation visit, patient assessment report 5800 can show only the GPI Report 1900-1 and episode summary 3512-1 for data collected before the visit, since no intervention has yet occurred.

One or more textual summaries of the patient's assessments can be located beneath episode summaries 3512. Region 5802 can include a text-based assessment of the patient's glucose patterns before intervention (e.g., overnight lows, highs after breakfast, etc.). These assessments can describe icons in region 1904-1 that have an elevated severity (e.g., over a threshold), or those assessments that have the relative highest severities.

Region 5804 can include a textual description of the actions that were initiated during the last prior intervention. Listed first is a description of the therapy change that was directed by an HCP with the authority to alter patient medication types and dosages. This therapy change would be recorded in the patient's database during the previous visit. A common therapy change is the reduction of insulin coverage in order to deal with excessive hypoglycemia, as reflected in the assessment. The patient's previously determined action plan is described next, particularly the issue being addressed (e.g., highs after breakfast), the behavior to address (e.g., breakfast cereal causing highs), and the action to be taken (e.g., consume eggs and toast instead of cereal). Again, this information would be recorded in the patient's database from the previous visit. Finally within region 5804 is a description of the EIS settings, which indicates the software configuration set during the previous visit.

As described herein, the EIS (or the software of the analyte monitoring system) includes software instructions to calculate various metrics from data taken at any time. These instructions can calculate metrics before and after each prior intervention and perform analysis and comparison. Based on this analysis, region 5806 can include a textual description of the assessment after intervention corresponding to each of region 5802's assessments before intervention.

For example, the first assessment in region 5802 indicated the occurrence of “overnight lows” as an identified pattern, for which a therapy change was identified (see region 5804). The EIS can select one or more appropriate metric types based on this identified pattern and/or the intervention recorded, and perform calculations of the values for those metric types to compare them before and after the intervention. Examples of metric types relevant to overnight lows can include the number of low glucose episodes occurring during overnight hours, and/or overnight TB70. The EIS can compare the calculated values for those metrics in the “before intervention” and “after intervention” periods and output the comparison. Alternatively, or in addition, the EIS can output one or more indications of how the identified pattern has changed (e.g., improved, worsened, generally unchanged, etc.), and/or whether the intervention was successful and/or by how much (e.g., very successful, moderately successful, moderately failed, severely failed, etc.).

In region 5806 of FIG. 58 the change in average overnight TB70 values is displayed from the “before intervention” period to the “after intervention” period (e.g., 154 to 104 minutes, indicating a reduction by 50 minutes) along with a visual (e.g., textual, symbolic, etc.) indication that the intervention was successful (e.g., “Success! Therapy change appears to have reduced overnight lows, though they still persist”). Such visual indications will vary based on the assessment and directed therapy change or action.

By way of a second example, the second assessment in region 5802 indicated the occurrence of glucose “highs after breakfast” as an identified pattern, for which a patient action plan was derived (see region 5804). Examples of metric types relevant to highs after breakfast can include post-breakfast average glucose values, and/or the number or average number of high glucose episodes occurring in the post-breakfast time period. Here, in region 5806, the change in average glucose values in post-breakfast time periods from the “before intervention” period to the “after intervention” period are displayed (e.g., 198 to 152 mg/dL, indicating a decrease by 46), as well as the change in high glucose episodes per week (or average episodes per week) from the “before intervention” period to the “after intervention” period (e.g., 7 to 5 per week, indicated a decrease by 2), along with a visual indication that the intervention was successful (e.g., “Success! Action plan seems to have reduced breakfast glucose highs and variability”).

The visual indication as to whether the intervention was successful can be determined by the EIS based on: (a) one or more patterns identified; (b) the recorded intervention; (c) one or more relevant metric changes, and (d) whether or not the one or more metric changes are sufficient to cross a threshold (e.g., a goal threshold) (in some embodiments there may be multiple thresholds, each indicating a different degree of success of failure, whether a goal was met, etc.). The EIS can refer to an array, list, table, or set of software instructions that correlate each of items (a)-(d) to various visual indications, such as the output texts shown in FIG. 58.

In region 5816 report 5800 can display an indication of any issues that remain from before the intervention, or any new issues that have arisen after the intervention, and can be similar to the display of issues in the before intervention region 5802. Region 5816 can also include a display of one or more suggested treatments, suggested changes to treatment, suggested actions, and/or suggested changes to EIS settings (e.g., prompt settings, detection thresholds, etc.) to address the respective issue. For assessments that indicate low glucose, suggested treatments or actions can correspond to those that may be generated or displayed when an icon 1916 (see FIG. 19B) from a GPI report indicates low glucose, such as shown in region 1906 of FIGS. 19A-19B. Suggestions for changes to EIS settings can be based on the underlying analyses for determining the value for each icon 1916, for example, analyses determining times at which for hypoglycemia (low glucose) risk, or variability is high. Further, one or more compliance metrics, before and after intervention, can be displayed in region 5826 of report 5800, such as the number of readings (e.g., scans) of the sensor control device taken over a time period (e.g., hour, day, week, etc.), the number or percentage of identified episodes to which at least one response was entered by the user (e.g., through a pick list), the number or percentage of responses given for each identified episode, the number of note entries made in the time period, the number or percentage of times the determined action was taken, etc.

In this embodiment, region 5808 displays an indication of the most frequent episode that has occurred, along with one or more of the most frequent responses entered by the user in response to the occurrence of episodes of that type. Here for example, the episode of glucose highs and/or variability after lunch (e.g., “lunch”) is indicated along with the most frequent responses that the bolus was too large or the meal was too small. The day selected for the report can also be required to have one of the most frequent responses as well as the “lunch” episode. Adjacent to this is a user selectable field 5818, selection of which can cause the display (e.g., as a pop up, new window, etc.) of a report for a day that meets the criterion of having a “lunch” episode, such as daily report 1800 described with respect to FIG. 18A, or a response report 4700 described with respect to FIGS. 48-49. As with reports 1800 and 4700, the report linked to from field 5818 can show a graph (e.g., 1806) including one or more of a glucose trace and color-coded markers for episodes occurring during the time period plotted. The linked report can also include a table (e.g., 1807) listing and describing each episode occurring during that time period.

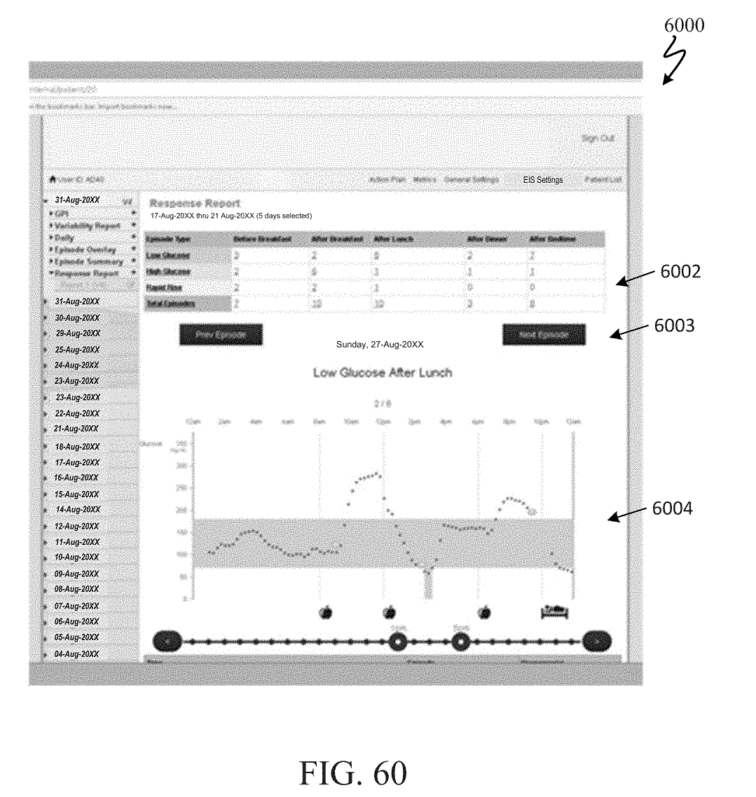

The EIS can default to display of the most recent day in which these one or more criteria are met, and the user can navigate between days meeting these criteria using daily navigation buttons or fields (e.g., 1802 or 4706). FIG. 60 depicts an example embodiment of the linked report 6000 for the episodic type “low glucose after lunch” having a table 6002 displaying an example of the number of episodes of each time that have occurred during the timeframe (e.g., post-intervention or longer) along with a graph 6004 of the patient's glycemic data during the time that the most recent “low glucose after lunch” episode occurred. Navigation between days where “low glucose after lunch” episodes occurred can be accomplished with fields 6003. The EIS can also present user selectable fields or buttons that allow the navigation forward and back across sequential days that do not necessarily meet the one or more criteria. Review of the daily plots for the most frequent episode and response can present a convenient and efficient mechanism for the HCP and the patient to discuss the most frequent issue that is causing glucose excursions, and devise a treatment or an action plan specifically to address the issue.

After the HCP and patient review information presented thus far, they can then devise the next intervention. The HCP may make a therapy change based on the latest assessment and guidance provided in region 5816 of report 5800. Selection of field 5832 can cause the display of a screen (e.g., pop up, new window, etc.) that shows the elements of the patient's therapy. For instance, if the patient is on fixed rapid acting insulin bolus doses and a single basal dose, and the HCP wants to record that they would like the patient to change their basal dose, then the HCP can select field 5832, and the EIS will display a user editable screen (not shown) that includes the elements of the patient's therapy. When the HCP makes a change to one of these elements, e.g., lowering the basal dose, the HCP can enter new text describing the therapy change, which the EIS can reference in field 5833 and also in prior intervention region 5804 upon the next patient visit. Alternatively, based on the change indicated by the HCP, the EIS can reference the appropriate standard text (e.g., by referring to an array, list, table, or set of software instructions) corresponding to that change, and use that standard text with programmable fields to indicate previous and new values (e.g., “10” units and “8” units as shown in field 5833. This standard text can be used for the prior intervention region 5804 upon the next patient visit.

The HCP and patient may also, or alternatively, alter or initiate an action plan as described earlier herein. Selection of user selectable field 5834 can cause the display of a screen (e.g., a pop up or new window, etc.) where action plan fields can be edited, such as screen 5100 described with respect to FIG. 51A. Beneath field 5834 are fields labeled “Issue to address,” “Behavior to address,” and “Patient Action.” These fields can be displayed for each issue to which an action plan is in effect. The Issue to address field can be automatically filled from the action plan (e.g., screen 5100).

The “Behavior to address” and “Patient Action” fields can be auto-filled with the appropriate text (e.g., “Occasional high carb lunches” and “Eat consistent amt carbs every lunch”) as determined by the user's completion of the action plan (e.g., screen 5100 of FIG. 51A). bae user selectable and, when clicked on, can cause the display (e.g., as a pop up or new window, etc.) of a list of selectable text fields. The selectable text fields are identified and included within the list based on the patient's glycemic pattern, predominant episode type(s), and predominant response(s) indicated for the time-of-day of the prior EIS setting. Selection of one of the selectable text fields will replace the text in the “Behavior to address” and “Patient Action” fields. After selection, the user (HCP, patient, etc.) can then edit the contents of the field if desired. Alternatively, the user can manually type in their own text directly without using the predetermined text strings from the list. If the EIS has action plan functionality, then that action plan can be adjusted accordingly based on the chosen content of the “Behavior to address” and “Patient Action” fields.

Report 5800 can also include a user selectable field 5836 that, upon selection, causes display of a screen that permits modification of EIS settings, such as the screens (or tabs) and/or settings described with respect to FIGS. 35A-35F. For example, modifying settings can allow the user to customize when notifications of episodes are generated and for which episode types, the various response types that can be displayed for selection by the user, the episode detection sensitivity, and/or others. User selectable field 5837 (e.g., labelled “Use suggested settings”) is also displayed here, selection of which adopts the setting(s) determined by the EIS. The suggested setting or settings can be shown in field 5840, and in this embodiment the EIS suggested setting is to prompt the patient when high glucose episodes occur in the time period after lunch (“Prompt for highs after lunch”). The suggested setting(s) can be based on the glucose metrics or episode metrics. For instance, one or more metrics that indicate that high glucose after lunch (“highs after lunch”) are a problem can correlate to the “Prompt for highs after lunch” suggestion. The metrics that could drive this suggestion, for example, could be that the central tendency (e.g., mean or median) glucose for the time period indicated as post-lunch is higher than all other time periods or higher than a threshold value. Alternatively, as an example, the suggestion could be based on an analysis that showed that the post-lunch period had higher glycemic variability or more frequent detected high episodes than the other time periods.

In one embodiment, the EIS automatically disables episode notifications when an action plan is initiated or changed. This is so that the prompting of the user to enter responses at reader 120 (e.g., the smartphone) will stop, allowing the patient to solely focus on the action plan reminders. In another embodiment, the notifications are not automatically disabled but rather a query to the user (e.g., a pop up) is generated requesting user confirmation that notifications should be enabled, explicitly giving the user the option.

In some embodiments when the EIS notifications are enabled, a query to the user is generated providing the option to disable action plan reminders. This can be for situations where the user (e.g., patient) is troubleshooting by engaging with the EIS response prompts or intervening according to the patient's action plan reminders, but not at the same time. This exclusion or inclusion can be optional or enforced, depending on the embodiment. In one embodiment, when the EIS notifications are enabled, a query to the user (either patient or HCP) is generated providing them the option to disable the action plan reminders. In another embodiment, when the EIS notifications are enabled, the action plan reminders are automatically disabled, without action by the user.

Report 5800 can also display an automatically generated referral. The referral can be generated by correlating various in inputs such as pattern guidance, predominant episode types and responses, with appropriate referrals. Alternatively, report 5800 could display a direct link to training media related to the inputs.

In some embodiments, the EIS can analyze the patient's glucose data and data collected from an insulin (or other drug) delivery device to determine if insulin meal bolus timing is appropriate. The mobile platform can process the repeated glucose data readings based on a meal detection process, such as those described in any of U.S. Patent Publication Nos. 2013/0085358, 2014/0350369 or 2014/0088393, or in Int'l Publ. No. WO 2015/153482, all of which are incorporated herein in their entirety and for all purposes. The meal detection process (or meal event detector) can be an algorithm, routine, or other set of instructions (part of or separate from the meal monitor application) that can detect and/or quantify the occurrence of an actual or potential meal event in the individual's monitored analyte data. The meal detection algorithm can provide an estimated meal start time for use by the EIS. The time of meal bolus delivery (e.g., a timestamp), which can be provided to the EIS by the insulin delivery device, or otherwise input to the EIS, can be compared to the meal start time. If the most recent (e.g., nearest) bolus timestamp was more than a threshold time limit (e.g., 30 minutes) before the meal start time, and/or after a threshold time limit (e.g., 10 minutes) from the meal start time, then the mobile platform can automatically classify the occurrence as a meal timing episode. Such episodes can be processed in the EIS like other glycemic episodes.

### Example Embodiments of Episode Investigative Software (EIS) on a Sensor Control Device

Also described herein are example embodiments of a sensor control device 102 capable of performing at least the episode detection portion of the EIS. Sensor control device 102 would also have the algorithm act programming to convert raw analyte data collected by sensor 104 into values readily indicative of the patient's glucose level (e.g., such as by the application of calibration and/or temperature compensation to the raw data, etc.). In these embodiments, sensor control device 102 would communicate detected episodes to reader device 120 along with glucose data before or after the episode, or in the peri-episodal time period surrounding that detected episode, such as for example a number of hours before (e.g., 2 hours) and a number of hours after (e.g., 6 hours) the detected episode.

In some of these embodiments (not all), sensor control device 102 would only communicate this information and not real-time glucose data obtained in the absence of a detected episode. In other words, sensor control device 102 would not provide glucose data to reader device 102 unless an episode is detected. This could be advantageous for commercial and regulatory purposes.

Referring back to the capability of performing episode detection on sensor control device 102, memory 253 (see FIG. 1C) can store at least episode detection instructions of the EIS and those instructions can be executed by processing circuitry 256. In some embodiments, episode detection can be performed every time new data is acquired by sensor electronics 250 from sensor 104. In other embodiments the episode detection can be performed whenever sensor control device 102 receives a request for data from reader device 120 (e.g., such as by an NFC scan request or Bluetooth request).

Similarly, information transfer regarding detected episodes from sensor control device 102 to reader device 120 can occur whenever an episode is detected if sensor control device 102 includes asynchronous data transmission capability, and/or whenever sensor control device 102 is queried or scanned by reader device 120. If sensor control device 102 has asynchronous data transmission capability, then sensor control device 102 can periodically perform an asynchronous transmission to reader device 120 to confirm the existence of a data communication link between the two devices (e.g., a Bluetooth pairing) and, in embodiments where sensor control device 102 collects and reports analyte data without detection of an episode therein, this a synchronous transmission can also include that analyte data.

Transmission of information pertaining to a detected episode (e.g., episode data) can include an array of the detected episode type and an associated time stamp of the start of the detected episode. The information transmission can also include an array of glucose values and associated time stamps, from a peri-episodal time period extending significantly before the episode (e.g., 2 hours) and ending significantly after the episode (e.g., 6 hours). In some embodiments, the most recent period of date (e.g., 30 minutes) may be excluded from the information transmission. In the opposite direction, information transfer from reader device 120 to sensor control device 102 can include the original episode detector sensitivity settings or any updates thereto.

In many embodiments, the episode detector present on the sensor control device 102 can be configured to detect all types of episodes at all times of the day. The portion of the EIS resident on reader device 120 can be configured to apply any filtering of episode type and time-of-day when determining whether to present a prompt to the patient. Sensor control device 102 would have the algorithmic processing capability to render actual glucose values from the raw data collected from the in vivo sensor 104.

The episode data transmitted to and received by reader device 120 can then be filtered to identify whether one or more of the detected episodes meet a subject criteria set on the reader. These criteria can be, for example, those settings set with selectable fields 3511 described with respect to FIG. 35B, which indicate the episode types and times-of-day for which to generate a notification to the subject, or a request to the subject, to enter episode information about the detected episode (e.g., information of the type described with respect to FIG. 35C). The subject criteria can also include, for example, the episode sensitivity levels (see FIG. 35F). If multiple episodes are detected in the data, then the request can be displayed for information about only the most recent episode. Reader 120 can then collect the entered episode information from the subject, store it locally and/or upload it to the server (e.g., trusted computer system 180), where it can be made available to the dual access platform.

In another example embodiment, reader device 120 can be configured to transfer additional EIS settings to the episode detector in the sensor control device 102, which would only detect episode types it was configured for, during the selected time-of day. In some embodiments reader device 120 can be configured to store or download additional information, such as responses and notes, on sensor control device 102. This would allow sensor control device 102 to be used with multiple reader devices 120, in that it could share this information between devices 120. Alternatively, this info could be stored and shared on the server.

Example Embodiments of Episode Investigative Software (EIS) with Expert System Capabilities

There has been interest in the use of expert systems or machine learning (referred to collectively herein as “expert systems”) to analyze data of diabetes patients and generate therapy recommendations therefrom. For example, an architecture with expert system capabilities has been proposed (e.g., Medtronic and IBM Watson collaboration) where the architecture can have access to various types of data provided by patients (e.g., insulin pump delivery systems, glucose monitoring systems), along with access to other data (e.g., meal logs, activity monitors, heart rate monitors, location) to discover or learn correlative patterns that can be exploited to provide a personalized system that detects potential problems and warns or guides patients into actions that can prevent the problem from occurring.

While this is feasible to some degree, it relies on the assumptions that patients will be willing to log meal information or can provide meal information with appropriate accuracy. Experience has shown that patients who carb count are not very successful at accurately estimating their carb intake. Also, carb counting in itself is inadequate to determine an accurate amount of insulin to deliver because other factors such as protein content and fat content also have an impact on glycemic response to meals. This feasibility is also based on the assumption that the patients will be willing to accommodate or pay for a high level of instrumentation connected to their bodies, or would be willing to share this information.

Embodiments are described herein that utilize the contextual information provided by the EIS along with glucose sensor data, and potentially other sources of information, as inputs to an expert system. As already described, the EIS detects problematic glycemic episodes and asks patients to provide contextual information about these episodes. For most patients, this is preferable to proactively entering contextual information (for example, information about meals, activity, medication, etc.) regardless of whether their glucose values are, or have recently been, excursive. A benefit of the EIS is that the patient is only asked to enter information when something significant occurs within their glucose patterns.

For example, the EIS can detect glycemic episodes such as high glucose, low glucose, rapid-rise in glucose and rapid-fall in glucose. In some embodiments, the EIS mobile platform reader 120 (e.g., smartphone app) includes an episode detector software module that processes the most recent glucose data collected at the sensor control device 102 to detect if any of these episode patterns have occurred. If an episode has been detected, then reader 120 will request that the patient respond to questions about the episode, e.g., as described herein with respect to FIGS. 12A-12C. For instance, if a high glucose episode was detected, reader 120 can ask the patient what they think may have caused the low glucose episode; the patient can select from a predefined list of answers, for instance, “high carb meal,” “missed insulin dose,” “lack of exercise today,” etc. Other questions may be asked, such as details about meals where the information can be collected in multiple forms such as text field entry, photograph, a pick list of common meals, a meal library, etc. In another example, a low glucose episode may be detected, and similarly questions could be asked such as what the cause is thought to be. Reader 120 could provide in this case a pick list of possible responses to this question, such as “low carb meal,” “more activity today than usual,” “alcohol consumption,” “illness,” etc.

Furthermore, reader 120 can also allow the patient or HCP to be able to customize the predefined questions and responses in order to tailor to be most relevant for that patient. These response lists provide repeatedly-selected categories of contextual information in which software programs can determine patterns related to these categories. The EIS can include reports that aggregate the episode and response information to facilitate the ability of patients and HCP's to identify the most frequent reasons (that is, contextual information categories attributed to causing episodes) for episodes, so that the HCP can focus their discussions with and recommendations for patients around behavior changes that are most likely to help the patient manage their glucose levels. A practical benefit of the EIS is that it doesn't require the patient to be diligent about always entering information, but rather only asks for information when there is a glycemic problem.

This information can be further used by embodiments of an expert system to analyze how the patient's glucose patterns respond to various contextual information content (and possibly other sensor or input data), and define relationships that can be used to warn or guide the patient's actions to prevent future adverse glycemic episodes.

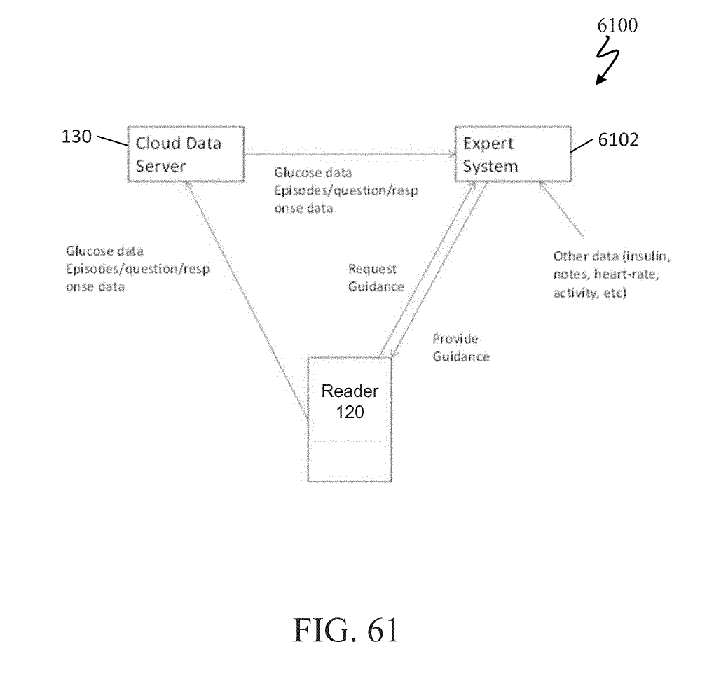

FIG. 61 is a conceptual block diagram depicting an example embodiment of a combined EIS-expert system 6100 having a reader 120, an expert system 6102, and a server 130. Reader 120 collects glucose data from sensor control device 102 (not shown). In many embodiments, this collected glucose data is uploaded to server 130 (e.g., the cloud). Server 130 can communicate this data to expert system 6102, which also can retrieve other data pertaining to the patient. Expert system 6102 includes software instructions stored on non-transitory memory (not shown) that, when executed by processing circuitry (not shown) of expert system 6102, runs a model for the patient that relates all of the inputs to future glucose levels. As more data becomes available, expert system 6102 updates the model and some of the relationships in the model become certain enough that they can be used to predict future glucose levels for that patient based on certain values of the inputs. Once these relationships are deemed to be adequately certain, then expert system 6102 can provide recommendations to the EIS mobile platform executed, in part, on reader 120. In some embodiments, the user can request recommendations from expert system 6102 via reader 120. In other embodiments, expert system 6102 periodically checks the inputs to look for predictions of adverse future glucose levels, and pro-actively pushes a notification to reader 120. Reader 120 can initiate a notification (alarm) to the patient or may provide a notification to the patient next time they view the EIS software on reader 120.

Other configurations of the combined EIS-expert system 6100 can also be implemented. For example, expert system 6102 can be integrated into reader 120 and server 130 could be omitted. In an alternative embodiment reader 120 could communication directly with expert system 6102 bypassing server 130.

Many types of relationships can be learned by system 6100 (or 6102). For instance, system 6100 could learn that when a particular patient entered “alcohol consumption” and “higher activity today” as responses to episodes when prompted by the EIS, this patient's glucose levels overnight tended to be lower for the next 12 hours. The system could learn, e.g., the relationship between these entries and certain glucose patterns would likely result in am overnight glucose level in the hypoglycemia range. This can be learned by system 6100 by having glucose data and contextual information available as inputs. Whenever new data is available, system 6100 can update the relationship models to iteratively and eventually arrive at a degree of confidence in the models high enough that recommendations to the patient or HCP can be made.

Aspects of the models used in expert systems are known by those skilled in the art. Focus here is placed on methods and systems for acquiring information to be used by the expert system and how the results from the expert system can be output to the patient.

The embodiments described herein are reiterated and supplemented in the following paragraphs which do so without explicit reference to the figures. In many embodiments described herein, an analyte monitoring system if provided for use in episode detection and information gathering. The system can included (a) a sensor control device having a sensor adapted for insertion into the body of a subject and adapted to measure signals indicative of a subject's analyte level over a time period, first processing circuitry, first non-transitory memory having stored thereon a first plurality of instructions that, when executed, causes the first processing circuitry to determine the analyte level of the subject over the time period from the measured signals, detect whether one or more analyte episodes occurred in the subject during the time period, and generate episode data describing the detected one or more analyte episodes, and first wireless communication circuitry adapted to communicate the episode data to the reader device. The system can also include (b) the reader device having second wireless communication circuitry adapted to receive the episode data from the sensor control device, second processing circuitry, and second non-transitory memory having stored thereon a second plurality of instructions that, when executed, causes the second processing circuitry to filter the episode data to identify whether one or more of the detected episodes meets a subject criteria and, if at least one of the detected episodes meets the subject criteria, to generate a request for the subject to provide episode information about the at least one detected episode, and a user interface adapted to display the generated request to the subject and receive the episode information from the subject.

In some embodiments, the second plurality of instructions, when executed, cause the second processing circuitry to, if two or more of the detected episodes meets the subject criteria, determine a most recent detected episode that meets the subject criteria and generate a request for the subject to provide episode information about the most recent detected episode.

In some embodiments, the sensor control device is configured to transmit data indicative of the analyte level of the subject only when associated with a detected episode such that data indicative of the analyte level of the subject is not transmitted when unassociated with a detected episode.

In some embodiments, the system further includes (c) a server remote from the reader device, the server adapted to receive the subject criteria from a remote computer over a communication link, wherein the server includes third processing circuitry and third non-transitory memory having a third plurality of instructions stored thereon that, when executed, causes the third processing circuitry to download the subject criteria to the reader device. In some embodiments, the server can be adapted to receive the episode information from the reader device. In some embodiments, the reader device is a smartphone and the second plurality of instructions are part of a downloaded EIS app.

In many embodiments herein, a method is described of using an analyte monitoring system for episode detection and information gathering, where the method includes measuring signals, with a sensor of a sensor control device, from within a body of a subject indicative of the subject's analyte level over a time period, determining, with processing circuitry of the sensor control device, the analyte level of the subject over the time period from the measured signals, detecting, with the processing circuitry of the sensor control device, whether one or more analyte episodes occurred in the subject during the time period, transmitting, with wireless communication circuitry of the sensor control device, episode data describing the detected one or more analyte episodes to a reader device, filtering, with processing circuitry of the reader device, the episode data to identify whether one or more of the detected episodes meets a subject criteria, and if at least one of the detected episodes meets the subject criteria, then causing, with the processing circuitry of the reader device, the display of a request to the subject, on a user interface of the reader device, for episode information about the at least one detected episode.

In some embodiments, the method further includes, if two or more of the detected episodes meets the subject criteria, then determining, with the processing circuitry of the reader device, a most recent detected episode that meets the subject criteria, and causing the display of a request for the subject to provide episode information about the most recent detected episode.

In some embodiments, the sensor control device is configured to transmit data indicative of the analyte level of the subject only when associated with a detected episode such that data indicative of the analyte level of the subject is not transmitted when unassociated with a detected episode.

In some embodiments, the method further includes receiving, by a server remote from the reader device, the subject criteria from a remote computer, and downloading the subject criteria from the server to the reader device. Also, the method can include receiving, by the server, the episode information from the reader device.

In many embodiments described herein, an analyte monitoring system is provided for use in episode detection and information gathering, where the system includes a reader device adapted to obtain analyte data indicative of the analyte levels of a subject from a sensor control device comprising an in vivo sensor, wherein the reader device includes first processing circuitry and first non-transitory memory having a first plurality of instructions stored thereon that, when executed, causes the first processing circuitry to: detect whether one or more analyte episodes occurred in the subject based on the analyte data, collect episode information from the subject about the detected one or more analyte episodes, and cause analyte information indicative of the subject's analyte levels and the episode information to be uploaded to a server, and the server includes second processing circuitry and second non-transitory memory having stored thereon a second plurality of instructions that, when executed, causes the second processing circuitry to: generate a displayable GUI and a plurality of reports accessible to a user by way of the GUI, and permit the user to associate uploaded analyte information from a first time period with a first visit between the subject and an HCP and associate uploaded analyte information from a second time period with a second visit between the subject and the HCP.

In some embodiments, the second plurality of instructions, when executed, cause the second processing circuitry to generate a first plurality of reports for the first visit and a second plurality of reports for the second visit. The GUI can include a visit guide with a first visit section including links to the first plurality of reports and a second visit section including links to the second plurality of reports. The second plurality of instructions, when executed, can also cause the second processing circuitry to permit the user to add an additional visit section for an additional visit to the visit guide and to associate uploaded analyte information from an additional time period with the additional visit. Further, the second plurality of instructions, when executed, can cause the second processing circuitry to, upon associating uploaded analyte information from the additional time period with the additional visit, automatically generate an additional plurality of reports for the additional visit and to link the additional plurality of reports in the additional visit section within the visit guide.

In some embodiments, the first plurality of reports includes a first report of a first type and wherein the second plurality of reports includes a second report of the first type, and the second plurality of instructions, when executed, cause the second processing circuitry to, in response to an indication from the user, generate the GUI with the first report adjacent to the second report for comparison by the user. The second plurality of instructions, when executed, can also cause the second processing circuitry to generate the GUI with the first report of the first type and a selectable field that permits selection of another report of the first type for comparison.

In some embodiments, the GUI can include a visit guide with a first visit section for the first visit and a second visit section for the second visit. The first visit section can include a first textual script that guides the HCP through a series of first topics for discussion with the subject during the first visit and the second visit section can include a second textual script that guides the HCP through a series of second topics for discussion with the subject during the second visit. In some embodiments, the first topics are different from the second topics. Also, the second plurality of instructions, when executed, can cause the second processing circuitry to generate an action plan control interface in the GUI, where the visit guide includes a user selectable link to the action plan control interface. The second visit section of the visit guide can include the user selectable link to the action plan control interface.

In some embodiments, the second plurality of instructions, when executed, can cause the second processing circuitry to generate an action plan control interface in the GUI. The action plan control interface can enable the user to record an action to be taken by the subject in response to detection of an episode. Also, the second plurality of instructions, when executed, can cause the second processing circuitry to cause the download of the recorded action to the reader device. The first plurality of instructions, when executed, can cause the first processing circuitry to generate a query for display to the subject as to whether the recorded action was taken.

In some embodiments, the action is associated with an episode of a first type, and the first plurality of instructions, when executed, causes the first processing circuitry to, after detection of the episode of the first type, generate a query for display to the subject as to whether the associated action was taken. The first plurality of instructions, when executed, can also cause the first processing circuitry to cause storage of a response to the query received from the subject, and cause the first processing circuitry to cause the stored response to be uploaded to the server as part of the episode information. The second plurality of instructions, when executed, can cause the second processing circuitry to generate a metric report including a metric indicative of a number of instances the action was taken by the subject, and a metric indicative of an efficacy of the action.

In some embodiments, the first plurality of instructions, when executed, causes the first processing circuitry to generate a reminder to the subject to take the recorded action, in some case prior to detection of the episode. The user can be the subject (or patient or diabetic) or the HCP.

In some embodiments, the first plurality of instructions, when executed, causes the first processing circuitry to: prompt the subject for a potential cause of the detected one or more episodes, and cause storage of a response to the prompt by the subject. The plurality of reports can include a response report, where the response report includes a table including a plurality of potential causes, a plurality of times of the day, and a plurality of response quantities, where each response quantity in the plurality corresponds to one of the plurality of potential causes and one of the plurality of times of the day. The response report can be displayable on the GUI such that each response quantity is a user selectable field.

In some embodiments, the second plurality of instructions, when executed, causes the second processing circuitry to, upon receipt of a selection of one of the plurality of response quantities corresponding to a first one of the plurality of potential causes and a first one of the plurality of times of the day, generate the displayable GUI with the table and a graph of analyte levels of the subject including a detected episode corresponding to the first one of the plurality of potential causes and the first one of the plurality of times of the day.

In many embodiments described herein, a method of episode detection and evaluation is provided, the method including: obtaining, with a reader device, analyte data indicative of the analyte levels of a subject from a sensor control device including an in vivo sensor, detecting, with the reader device, whether one or more analyte episodes occurred in the subject based on the analyte data, collecting, with the reader device, episode information from the subject about the detected one or more analyte episodes, causing, with the reader device, analyte information indicative of the subject's analyte levels and the episode information to be uploaded to a server, generating, with the server, a displayable graphical user interface (GUI) and a plurality of reports accessible to a user by way of the GUI, and permitting, with the server, a user to associate uploaded analyte information from a first time period with a first visit between the subject and a health care provider (HCP) and associate uploaded analyte information from a second time period with a second visit between the subject and the HCP.

In some embodiments, the method includes displaying the GUI on a computer terminal remote from the server. The method can include generating, by the server, a first plurality of reports for the first visit and a second plurality of reports for the second visit. The GUI can include a visit guide with a first visit section including links to the first plurality of reports and a second visit section including links to the second plurality of reports. The method can include permitting, by the server, the user to add an additional visit section for an additional visit to the visit guide and to associate uploaded analyte information from an additional time period with the additional visit. The method can also include, upon associating uploaded analyte information from the additional time period with the additional visit, automatically generating, by the server, an additional plurality of reports for the additional visit and to link the additional plurality of reports in the additional visit section within the visit guide.

In some embodiments, the first plurality of reports includes a first report of a first type and wherein the second plurality of reports includes a second report of the first type, the method including, in response to an indication from the user, generating, by the server, the displayable GUI with the first report adjacent to the second report for comparison by the user. The method can include generating, with the server, the displayable GUI with the first report of the first type and a selectable field that permits selection of another report of the first type for comparison.

In some embodiments, the GUI includes a visit guide with a first visit section for the first visit and a second visit section for the second visit, and the first visit section includes a first textual script that guides the HCP through a series of first topics for discussion with the subject during the first visit and the second visit section includes a second textual script that guides the HCP through a series of second topics for discussion with the subject during the second visit. The first topics can be different from the second topics.

In some embodiments, the method can include generating, with the server, an action plan control interface in the displayable GUI, wherein the visit guide includes a user selectable link to the action plan control interface. The second visit section of the visit guide can include the user selectable link to the action plan control interface.

In some embodiments, the method includes generating, by the server, an action plan control interface in the displayable GUI, recording, by the server, an action to be taken by the subject in response to detection of an episode, wherein the action is entered by the user by way of the action plan control interface, and downloading the recorded action to the reader device. The method can also include generating, by the reader device, a query for display to the subject as to whether the recorded action was taken. The action can be associated with an episode of a first type, and the method can include, after detection of the episode of the first type, generating, by the reader device, a query for display to the subject as to whether the associated action was taken.

The method can include storing, by the reader device, a response to the query received from the subject and uploading, by the reader device, the stored response to the server as part of the episode information. The plurality of reports can include a metric report including a metric indicative of a number of instances the action was taken by the subject and/or a metric indicative of an efficacy of the action.

In some embodiments, the method includes generating, by the reader device, a reminder to the subject to take the recorded action. The method can include generating, by the reader device, the reminder prior to detection of the episode.

In some embodiments, the method includes prompting, by the reader device, the subject for a potential cause of the detected one or more episodes, and storing, by the reader device, a response to the prompt by the subject. The plurality of reports can include a response report. The response report can include a table including a plurality of potential causes, a plurality of times of the day, and a plurality of response quantities, wherein each response quantity in the plurality corresponds to one of the plurality of potential causes and one of the plurality of times of the day. The response report can be displayable on the GUI such that each response quantity is a user selectable field. The method can include, upon receipt of a selection of one of the plurality of response quantities corresponding to a first one of the plurality of potential causes and a first one of the plurality of times of the day, generating, by the server, the displayable GUI with the table and a graph of analyte levels of the subject including a detected episode corresponding to the first one of the plurality of potential causes and the first one of the plurality of times of the day.

In many embodiments described herein, an analyte monitoring system is provided for use in episode detection and information gathering, the system including: a reader device adapted to obtain analyte data indicative of the analyte levels of a subject from a sensor control device including an in vivo sensor, where the reader device includes first processing circuitry and first non-transitory memory having a first plurality of instructions stored thereon that, when executed, causes the first processing circuitry to: detect whether one or more analyte episodes occurred in the subject based on the analyte data, collect episode information from the subject about the detected one or more analyte episodes, and cause analyte information indicative of the subject's analyte levels and the episode information to be uploaded to a server. The system can also include the server which, in turn, includes second processing circuitry and second non-transitory memory having stored thereon a second plurality of instructions that, when executed, causes the second processing circuitry to: generate a displayable graphical user interface (GUI), a first plurality of reports for a first visit between the subject and a health care provider (HCP), and a second plurality of reports for a second visit between the subject and the HCP.

In some embodiments, the second plurality of instructions, when executed, causes the second processing circuitry to generate the first plurality of reports from uploaded analyte information from a first time period and to generate the second plurality of reports from uploaded analyte information from a second time period different from the first time period. In some embodiments, the first and second time periods do not overlap.

The first plurality of reports can include a first report of a first type and the second plurality of reports can include a second report of the first type, and where the second plurality of instructions, when executed, causes the second processing circuitry to, in response to an indication from the user, generate the displayable GUI with the first report adjacent to the second report for comparison by the user. The second plurality of instructions, when executed, can cause the second processing circuitry to generate the displayable GUI with the first report of the first type and a selectable field that permits selection of another report of the first type for comparison.

In many embodiments described herein, a method of episode detection and evaluation is provided that includes: obtaining, with a reader device, analyte data indicative of the analyte levels of a subject from a sensor control device including an in vivo sensor, detecting, with the reader device, whether one or more analyte episodes occurred in the subject based on the analyte data, collecting, with the reader device, episode information from the subject about the detected one or more analyte episodes, causing, with the reader device, analyte information indicative of the subject's analyte levels and the episode information to be uploaded to a server, and generating, with the server, a displayable graphical user interface (GUI), a first plurality of reports for a first visit between the subject and a health care provider (HCP), and a second plurality of reports for a second visit between the subject and the HCP.

In some embodiments, the method can include generating, with the server, the first plurality of reports from uploaded analyte information from a first time period and the second plurality of reports from uploaded analyte information from a second time period different from the first time period. In some embodiments, the first and second time periods do not overlap.

In some embodiments, the first plurality of reports includes a first report of a first type and the second plurality of reports includes a second report of the first type, the method further including, in response to an indication from the user, generating, with the server, the displayable GUI with the first report adjacent to the second report for comparison by the user. The method can include generating, with the server, the displayable GUI with the first report of the first type and a selectable field that permits selection of another report of the first type for comparison.

In many embodiments described herein, an analyte monitoring system for use in episode detection and information gathering is provided, where the system includes: a reader device adapted to obtain analyte data indicative of the analyte levels of a subject from a sensor control device including an in vivo sensor, where the reader device includes first processing circuitry and first non-transitory memory having a first plurality of instructions stored thereon that, when executed, causes the first processing circuitry to: detect whether one or more analyte episodes occurred in the subject based on the analyte data, collect episode information from the subject about the detected one or more analyte episodes, and cause analyte information indicative of the subject's analyte levels and the episode information to be uploaded to a server. The system can also include the server, which includes second processing circuitry and second non-transitory memory having stored thereon a second plurality of instructions that, when executed, causes the second processing circuitry to: generate a displayable GUI including a visit guide having a first section for a first visit between the subject and an HCP and a second section for a second visit between the subject and the HCP.

In some embodiments, the first visit section includes a first textual script that guides the HCP through a series of first topics for discussion with the subject during the first visit and a first plurality of links to reports generated from analyte data of the subject associated with the first visit. In some embodiments, the second visit section includes a second textual script that guides the HCP through a series of second topics for discussion with the subject during the second visit and a second plurality of links to reports generated from analyte data of the subject associated with the second visit. In some embodiments, the first topics are different from the second topics.

In many embodiments described herein, a method of episode detection and evaluation is provided, the method including: obtaining, with a reader device, analyte data indicative of the analyte levels of a subject from a sensor control device including an in vivo sensor, detecting, with the reader device, whether one or more analyte episodes occurred in the subject based on the analyte data, collecting, with the reader device, episode information from the subject about the detected one or more analyte episodes, causing, with the reader device, analyte information indicative of the subject's analyte levels and the episode information to be uploaded to a server, and generating, with the server, a displayable graphical user interface (GUI) including a visit guide having a first section for a first visit between the subject and a health care provider (HCP) and a second section for a second visit between the subject and the HCP.

In some embodiments, the first visit section includes a first textual script that guides the HCP through a series of first topics for discussion with the subject during the first visit and a first plurality of links to reports generated from analyte data of the subject associated with the first visit. The second visit section can include a second textual script that guides the HCP through a series of second topics for discussion with the subject during the second visit and a second plurality of links to reports generated from analyte data of the subject associated with the second visit. In some embodiments, the first topics are different from the second topics.

In many embodiments described herein, an analyte monitoring system for use in episode detection and information gathering is provided, where the system includes: a reader device adapted to obtain analyte data indicative of the analyte levels of a subject from a sensor control device including an in vivo sensor, wherein the reader device includes first processing circuitry and first non-transitory memory having a first plurality of instructions stored thereon that, when executed, causes the first processing circuitry to: detect whether one or more analyte episodes occurred in the subject based on the analyte data, collect episode information from the subject about the detected one or more analyte episodes, and cause analyte information indicative of the subject's analyte levels and the episode information to be uploaded to a server. The system can also include the server, which in turn can include second processing circuitry and second non-transitory memory having stored thereon a second plurality of instructions that, when executed, causes the second processing circuitry to: generate a displayable graphical user interface (GUI) including an action plan control interface, wherein the action plan control interface enables the user to record an action to be taken by the subject in response to detection of the one or more episodes.

In some embodiments, the second plurality of instructions, when executed, causes the second processing circuitry to cause the download of the recorded action to the reader device. The first plurality of instructions, when executed, can cause the first processing circuitry to generate a query for display to the subject as to whether the recorded action was taken. The action can be associated with an episode of a first type, and wherein the first plurality of instructions, when executed, causes the first processing circuitry to, after detection of the episode of the first type, generate a query for display to the subject as to whether the associated action was taken. In some embodiments, the first plurality of instructions, when executed, causes the first processing circuitry to cause storage of a response to the query received from the subject. The first plurality of instructions, when executed, can cause the first processing circuitry to cause the stored response to be uploaded to the server as part of the episode information. The second plurality of instructions, when executed, can cause the second processing circuitry to generate a metric report including a metric indicative of a number of instances the action was taken by the subject, and/or a metric indicative of an efficacy of the action.

In some embodiments, the first plurality of instructions, when executed, causes the first processing circuitry to generate a reminder to the subject to take the recorded action, in some case prior to detection of the episode.

In many embodiments described herein, a method of episode detection and evaluation is provided that includes: obtaining, with a reader device, analyte data indicative of the analyte levels of a subject from a sensor control device including an in vivo sensor, detecting, with the reader device, whether one or more analyte episodes occurred in the subject based on the analyte data, collecting, with the reader device, episode information from the subject about the detected one or more analyte episodes, causing, with the reader device, analyte information indicative of the subject's analyte levels and the episode information to be uploaded to a server, generating, with the server, a displayable GUI including an action plan control interface, and recording, by the server, an action to be taken by the subject in response to detection of the one or more episodes.

In some embodiments, the action is entered by the user by way of the action plan control interface. The method can include downloading the recorded action to the reader device and generating, by the reader device, a query for display to the subject as to whether the recorded action was taken. The action can be associated with an episode of a first type, and the method can include, after detection of the episode of the first type, generating, by the reader device, a query for display to the subject as to whether the associated action was taken and storing, by the reader device, a response to the query received from the subject. The method can include uploading, by the reader device, the stored response to the server as part of the episode information and generating, by the server, a metric report including a metric indicative of a number of instances the action was taken by the subject and/or a metric indicative of an efficacy of the action.

In many embodiments described herein, an analyte monitoring system for use in episode detection and analysis is provided, where the system includes: a reader device adapted to obtain analyte data indicative of the analyte levels of a subject from a sensor control device including an in vivo sensor, wherein the reader device includes first processing circuitry and first non-transitory memory having a first plurality of instructions stored thereon that, when executed, causes the first processing circuitry to: detect whether one or more analyte episodes occurred in the subject based on the analyte data, prompt the subject for a potential cause of the detected one or more episodes, cause storage of a response to the prompt by the subject, and cause analyte information indicative of the subject's analyte levels and episode information including the response to be uploaded to a server. The system can also include the server, which in turn includes second processing circuitry and second non-transitory memory having stored thereon a second plurality of instructions that, when executed, causes the second processing circuitry to: generate a displayable graphical user interface (GUI) and a response report accessible to a user by way of the GUI.

In some embodiments, the response report includes a table including a plurality of potential episode causes, a plurality of episode occurrence times, and a plurality of response quantities, wherein each response quantity in the plurality of response quantities corresponds to one of the plurality of potential episode causes and one of the plurality of episode occurrence times. The response report can be displayable on the GUI such that each response quantity is a user selectable field. The second plurality of instructions, when executed, can cause the second processing circuitry to, upon receipt of a selection of one of the plurality of response quantities corresponding to a first one of the plurality of potential episode causes and a first one of the plurality of episode occurrence times, generate the displayable GUI with the table and a graph of analyte levels of the subject including a detected episode corresponding to the first one of the plurality of potential episode causes and the first one of the plurality of episode occurrence times.

In many embodiments described herein, a method of episode detection and evaluation is provided that includes: obtaining, with a reader device, analyte data indicative of the analyte levels of a subject from a sensor control device including an in vivo sensor, detecting, with the reader device, whether one or more analyte episodes occurred in the subject based on the analyte data, prompting, with the reader device, the subject for a potential cause of the detected one or more episodes, storing, by the reader device, a response to the prompt by the subject, uploading to a server, by the reader device, analyte information indicative of the subject's analyte levels and episode information including the response, and generating, with the server, a displayable GUI and a response report accessible to a user by way of the GUI.

In some embodiments, the response report includes a table including a plurality of potential episode causes, a plurality of episode occurrence times, and a plurality of response quantities, wherein each response quantity in the plurality of response quantities corresponds to one of the plurality of potential episode causes and one of the plurality of episode occurrence times. The response report can be displayable on the GUI such that each response quantity is a user selectable field. The method can include, upon receipt of a selection of one of the plurality of response quantities corresponding to a first one of the plurality of potential episode causes and a first one of the plurality of episode occurrence times, generating, by the server, the displayable GUI with the table and a graph of analyte levels of the subject including a detected episode corresponding to the first one of the plurality of potential episode causes and the first one of the plurality of episode occurrence times.

In many embodiments described herein, a method of generating graphical user interfaces for use with an episode investigation software is provided that includes: generating, with a server, a scheduling interface for presentation as a first graphical user interface, where the scheduling interface comprises a calendar including a plurality of timeslots for patient visits with a health care professional (HCP), where timeslots having a scheduled visit visually indicate the type of scheduled visit; receiving, at the server, an indication of selection of one of the plurality of timeslots having a scheduled visit by a patient; and generating, with the server, an assessment report for presentation as a second GUI, wherein the assessment report includes information assessing the efficacy of an intervention for management of a glycemic condition of the patient.

In some embodiments, the type of scheduled visit is indicative of progress through an iterative glycemic episode investigation program. The type of scheduled visit can also or alternatively be selected from a group including: an initial visit, an initial evaluation visit, and an episode investigation software (EIS) evaluation visit. In certain embodiments, for each timeslot that indicates the type of visit as an EIS evaluation visit, the timeslot includes a visual indication of placement in an EIS evaluation visit sequence, and the visual indication of placement in the EIS evaluation visit sequence can be a number.

In some embodiments, the assessment report includes a first graph of glycemic patterns for the patient prior to the intervention and a second graph of glycemic patterns for the patient after the intervention. The assessment report can also, or alternatively, include a description of the intervention, an indication of degree of success of the intervention in treating a glycemic condition, an indication of a glycemic issue that exists after the intervention, a metric indicating degree of compliance with a requirement of an episode investigation software (EIS), a recommendation to address a glycemic issue that exists after the intervention, and/or a user selectable field for display of glycemic data collected for the patient that indicates the occurrence of a glycemic issue that exists after the intervention.

In some embodiments, the method further includes: receiving, at the server, an indication of selection of the user selectable field for display of glycemic data; and generating, with the server, a report with access to glycemic data collected for the patient that indicates the occurrence of the glycemic issue.

In some embodiments, the assessment report includes a user selectable field, and the method further includes: receiving, at the server, an indication of selection of the user selectable field; and generating, with the server, an action plan interface for presentation as a third GUI, wherein the action plan interface permits configuration of one or more elements of an action plan to address a glycemic issue that exists after the intervention.

In many embodiments described herein, a system for generating graphical user interfaces for use with an episode investigation software is provided that includes: a server including processing circuitry and non-transitory memory having stored thereon a plurality of instructions that, when executed, causes the processing circuitry to: generate a scheduling interface for presentation as a first graphical user interface, where the scheduling interface includes a calendar having a plurality of timeslots for patient visits with a health care professional (HCP), where timeslots having a scheduled visit visually indicate the type of scheduled visit; read a received indication of selection of one of the plurality of timeslots having a scheduled visit by a patient; and generate an assessment report for presentation as a second GUI, wherein the assessment report includes information assessing the efficacy of an intervention for management of a glycemic condition of the patient.

In some embodiments, the type of scheduled visit is indicative of progress through an iterative glycemic episode investigation program. The type of scheduled visit can also or alternatively be selected from a group comprising: an initial visit, an initial evaluation visit, and an episode investigation software (EIS) evaluation visit.

In some embodiments, for each timeslot that indicates the type of visit as an EIS evaluation visit, the timeslot includes a visual indication of placement in an EIS evaluation visit sequence. In certain embodiments, the visual indication of placement in the EIS evaluation visit sequence can be a number.

In some embodiments, the assessment report includes a first graph of glycemic patterns for the patient prior to the intervention and a second graph of glycemic patterns for the patient after the intervention. Also, or alternatively, the assessment report can include a description of the intervention, an indication of degree of success of the intervention in treating a glycemic condition, an indication of a glycemic issue that exists after the intervention, a metric indicating degree of compliance with a requirement of an episode investigation software (EIS), a recommendation to address a glycemic issue that exists after the intervention, and/or a user selectable field for display of glycemic data collected for the patient that indicates the occurrence of a glycemic issue that exists after the intervention.

In some embodiments, the plurality of instructions, when executed, further cause the processing circuitry to: read a received indication of selection of the user selectable field for display of glycemic data; and generate a report with access to glycemic data collected for the patient that indicates the occurrence of the glycemic issue.

In some embodiments, the assessment report comprises a user selectable field, and the plurality of instructions, when executed, further cause the processing circuitry to: read a received indication of selection of the user selectable field; and generate an action plan interface for presentation as a third GUI, wherein the action plan interface permits configuration of one or more elements of an action plan to address a glycemic issue that exists after the intervention.

The episode investigate software has been described herein in the context of an analyte monitoring system, with particular applicability to the monitoring of glucose to assist diabetics in managing their condition. However, functionality, attributes, reports, and other features of the episode investigative software have broader applicability to the as well, including to the management of other types of medical conditions and even to applications outside of the medical field.

For example, the subject matter disclosed herein is not limited to investigating the causes of episodes, but can be used to monitor and characterize other attributes, conditions, or characteristics of a person that may or may not qualify as an episode as that term is used in its broadest scope herein. The data collected and analyzed can be of virtually any type, such as activity related data (e.g., steps taken, calories burned, etc. as tracked and reported by devices such as an activity monitor), meal information, location information (e.g., with a GPS-enabled device), other forms of biometric data such as characteristics of the heart and/or vascular system (e.g., heart rate, blood pressure, heart sounds, etc.), characteristics of other organs such as the eye (e.g., intraocular pressure), skin (e.g., perspiration), brain (e.g., neuro-electrical activity), and others. This collected data can be compared to one or more rules, conditions, requirements, thresholds, etc., that are indicative of the occurrence of an episode, event, characteristic, symptom, precursor, activity, etc. that is relevant to the particular application.

Computer program instructions for carrying out operations in accordance with the described subject matter may be written in any combination of one or more programming languages, including an object oriented programming language such as Java, JavaScript, Smalltalk, C++, C#, Transact-SQL, XML, PHP or the like and conventional procedural programming languages, such as the “C” programming language or similar programming languages. The program instructions may execute entirely on the user's computing device, partly on the user's computing device, as a stand-alone software package, partly on the user's computing device and partly on a remote computing device or entirely on the remote computing device or server. In the latter scenario, the remote computing device may be connected to the user's computing device through any type of network, including a local area network (LAN) or a wide area network (WAN), or the connection may be made to an external computer (for example, through the Internet using an Internet Service Provider).

For every embodiment described herein, to the extent the capability is described for a user to provide input (e.g., by selecting a selectable field on a touchscreen of a reader device, by providing an input through a mechanical button or switch on a reader device, by selecting a field on a display with a mouse, to name a few) then the associated processing circuitry (e.g., that circuitry executing the software described herein) can be described and claimed as monitoring for that user input. This monitoring can include, for example, monitoring for the occurrence of an interrupt or notification from software associated with the hardware (touchscreen, mouse, etc.) accepting that user input. The processing circuitry can also have instructions that permit the processing circuitry to receive and/or read that user input to determine what selection was made by the user. Similarly, for every graphical user interface and/or screen that is displayed, the processing circuitry can be described and claimed as causing the display of (or generating) that graphical user interface or screen and each and every feature therein (icons, text, images, etc.). Although recognized elsewhere herein, it is reiterated that this processing circuitry can be a single processor chip or can be multiple processor chips or portions of processor chips distributed throughout the overall electronic device or devices that are in communication with each other.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

To the extent the embodiments disclosed herein include or operate in association with memory, storage, and/or computer readable media, then that memory, storage, and/or computer readable media are non-transitory. Accordingly, to the extent that memory, storage, and/or computer readable media are covered by one or more claims, then that memory, storage, and/or computer readable media is only non-transitory.

In many instances entities are described herein as being coupled to other entities. It should be understood that the terms “coupled” and “connected” (or any of their forms) are used interchangeably herein and, in both cases, are generic to the direct coupling of two entities (without any non-negligible intervening entities) and the indirect coupling of two entities (with one or more non-negligible intervening entities). Where entities are shown as being directly coupled together, or described as coupled together without description of any intervening entity, it should be understood that those entities can be indirectly coupled together as well unless the context clearly dictates otherwise.

As used herein and in the appended claims, the singular forms “a,” “an,” and “the” include plural referents unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

1. An analyte monitoring system for use in episode detection and information gathering, the system comprising:
(a) a sensor control device comprising:
a sensor adapted for insertion into the body of a subject and to measure signals indicative of a subject's analyte level over a time period;
first processing circuitry;
first non-transitory memory having stored thereon a first plurality of instructions that, when executed, causes the first processing circuitry to determine the analyte level of the subject over the time period from the measured signals, detect whether one or more analyte episodes occurred in the subject during the time period, and generate episode data describing the detected one or more analyte episodes; and
first wireless communication circuitry adapted to communicate the episode data to the reader device; and
(b) the reader device comprising;
second wireless communication circuitry adapted to receive the episode data from the sensor control device;
second processing circuitry; and
second non-transitory memory having stored thereon a second plurality of instructions that, when executed, causes the second processing circuitry to filter the episode data to identify whether one or more of the detected episodes meets a subject criteria and, if at least one of the detected episodes meets the subject criteria, to generate a request for the subject to provide episode information about the at least one detected episode; and
a user interface adapted to display the generated request to the subject and receive the episode information from the subject.

2. The analyte monitoring system of claim 1, wherein the second plurality of instructions, when executed, causes the second processing circuitry to, if two or more of the detected episodes meets the subject criteria, determine a most recent detected episode that meets the subject criteria and generate a request for the subject to provide episode information about the most recent detected episode.

3. The analyte monitoring system of claim 1, wherein the sensor control device is configured to transmit data indicative of the analyte level of the subject only when associated with a detected episode such that data indicative of the analyte level of the subject is not transmitted when unassociated with a detected episode.

4. The analyte monitoring system of claim 1, further comprising:
(c) a server remote from the reader device, the server adapted to receive the subject criteria from a remote computer over a communication link, wherein the server comprises third processing circuitry and third non-transitory memory having a third plurality of instructions stored thereon that, when executed, causes the third processing circuitry to download the subject criteria to the reader device.

5. The analyte monitoring system of claim 4, wherein the server is adapted to receive the episode information from the reader device.

6. The analyte monitoring system of claim 1, wherein the reader device is a smartphone and the second plurality of instructions are part of a downloaded episode investigation software (EIS) app.

7. A method of using an analyte monitoring system for episode detection and information gathering, the method comprising:
measuring signals, with a sensor of a sensor control device, from within a body of a subject indicative of the subject's analyte level over a time period;
determining, with processing circuitry of the sensor control device, the analyte level of the subject over the time period from the measured signals;
detecting, with the processing circuitry of the sensor control device, whether one or more analyte episodes occurred in the subject during the time period;
transmitting, with wireless communication circuitry of the sensor control device, episode data describing the detected one or more analyte episodes to a reader device;
filtering, with processing circuitry of the reader device, the episode data to identify whether one or more of the detected episodes meets a subject criteria; and
if at least one of the detected episodes meets the subject criteria, then causing, with the processing circuitry of the reader device, the display of a request to the subject, on a user interface of the reader device, for episode information about the at least one detected episode.

8. The method of claim 7, further comprising:
if two or more of the detected episodes meets the subject criteria, then determining, with the processing circuitry of the reader device, a most recent detected episode that meets the subject criteria; and
causing the display of a request for the subject to provide episode information about the most recent detected episode.

9. The method of claim 7, wherein the sensor control device is configured to transmit data indicative of the analyte level of the subject only when associated with a detected episode such that data indicative of the analyte level of the subject is not transmitted when unassociated with a detected episode.

10. The method of claim 7, further comprising:
receiving, by a server remote from the reader device, the subject criteria from a remote computer; and
downloading the subject criteria from the server to the reader device.

11. The method of claim 10, further comprising receiving, by the server, the episode information from the reader device.

12. The method of claim 7, wherein the reader device is a smartphone executing a downloaded episode investigation software (EIS) app.

13. The method of claim 7, wherein the analyte level is a glucose level.

14. An analyte monitoring system for use in episode detection and information gathering, the system comprising:
a reader device adapted to obtain analyte data indicative of the analyte levels of a subject from a sensor control device comprising an in vivo sensor, wherein the reader device comprises first processing circuitry and first non-transitory memory having a first plurality of instructions stored thereon that, when executed, causes the first processing circuitry to:
detect whether one or more analyte episodes occurred in the subject based on the analyte data;
collect episode information from the subject about the detected one or more analyte episodes; and
cause analyte information indicative of the subject's analyte levels and the episode information to be uploaded to a server; and
the server comprising second processing circuitry and second non-transitory memory having stored thereon a second plurality of instructions that, when executed, causes the second processing circuitry to:
generate a displayable graphical user interface (GUI) and a plurality of reports accessible to a user by way of the GUI, and
permit the user to associate uploaded analyte information from a first time period with a first visit between the subject and a health care provider (HCP) and associate uploaded analyte information from a second time period with a second visit between the subject and the HCP.

15. The analyte monitoring system of claim 14, wherein the second plurality of instructions, when executed, causes the second processing circuitry to generate a first plurality of reports for the first visit and a second plurality of reports for the second visit.

16. The analyte monitoring system of claim 15, wherein the GUI comprises a visit guide with a first visit section comprising links to the first plurality of reports and a second visit section comprising links to the second plurality of reports.

17. The analyte monitoring system of claim 16, wherein the second plurality of instructions, when executed, causes the second processing circuitry to permit the user to add an additional visit section for an additional visit to the visit guide and to associate uploaded analyte information from an additional time period with the additional visit.

18. The analyte monitoring system of claim 17, wherein the second plurality of instructions, when executed, causes the second processing circuitry to, upon associating uploaded analyte information from the additional time period with the additional visit, automatically generate an additional plurality of reports for the additional visit and to link the additional plurality of reports in the additional visit section within the visit guide.

19. The analyte monitoring system of claim 15, wherein the first plurality of reports comprises a first report of a first type and wherein the second plurality of reports comprises a second report of the first type, and wherein the second plurality of instructions, when executed, causes the second processing circuitry to, in response to an indication from the user, generate the GUI with the first report adjacent to the second report for comparison by the user.

20. The analyte monitoring system of claim 19, wherein the second plurality of instructions, when executed, causes the second processing circuitry to generate the GUI with the first report of the first type and a selectable field that permits selection of another report of the first type for comparison.

21-147. (canceled)
